(19) United States
(12) Patent Application Publication
(10) Pub. No.: US 2022/0315551 A1
(43) Pub. Date: Oct. 6, 2022

(54) 2-ALKYLCARBONYL[2,3-b]FURAN-4,9-DIONE PRODUCTION METHOD AND PRODUCTION INTERMEDIATE THEREFOR

(71) Applicant: SUMITOMO DAINIPPON PHARMA CO., LTD., Osaka (JP)

(72) Inventors: Yoko TAKAHASHI, Osaka (JP); Yusuke YAMAI, Osaka (JP)

(73) Assignee: SUMITOMO DAINIPPON PHARMA CO., LTD., Osaka (JP)

(21) Appl. No.: 17/616,909

(22) PCT Filed: Jun. 12, 2020
(86) PCT No.: PCT/JP2020/023198

§ 371 Date: Dec. 6, 2021

(30) Foreign Application Priority Data
Jun. 14, 2019 (JP) 2019-110987

(51) Int. Cl. C07D 307/92

(52) U.S. Cl. CPC C07D 307/92

(57) ABSTRACT

Provided is a method for producing a substance related to 2-alkylcarbonylnaphtho[2,3-b]furan-4,9-dione that is suitable for industrial production. The present disclosure provides: a method for producing two production intermediates for 2-alkylcarbonyl[2,3-b]furan-4,9-dione by reacting commercially available 2-hydroxy-1,4-naphthoquinone with equally inexpensive, commercially available induced N,N-substituted formamide dimethyl acetal, and further reacting the product with an inexpensive commercially available 2-halo-1,4-diketone compound in the presence of water; and a substance related to the same.

### TECHNICAL FIELD

The present disclosure relates to a method of manufacturing 2-alkylcarbonyl[2,3-b]furan-4,9-dione, which is useful as a pharmaceutical product, a manufacture intermediate thereof, and a method of manufacturing said manufacture intermediate.

### BACKGROUND ART

For example, the methods described in Patent Literature 1 are known as a method of manufacturing 2-acetylnaphtho[2,3-b]furan-4,9-dione or a method of manufacturing a related substance.

### CITATION LIST

### Patent Literature

[PTL 1] International Publication No. WO 2009/036099

### SUMMARY OF INVENTION

### Solution to Problem

The present disclosure provides a method of manufacturing 2-alkylcarbonylnaphtho[2,3-b]furan-4,9-dione, which is useful as a pharmaceutical product.

The present disclosure provides an excellent industrial manufacturing method for manufacturing 2-alkylcarbonylnaphtho[2,3-b]furan-4,9-dione, which is represented by formula (6), in a low number of steps without requiring a protecting group attaching/detaching step in a convenient, high yielding, and cost effective manner.

More specifically in one embodiment, a manufacture intermediate (3) was obtained by reacting a cost-effective and commercially available 2-hydroxy-1,4-naphthoquinone (1) with a cost-effective and commercially available N,N-substituted formamide dimethyl acetal (2a) or N,N-substituted formamide (2b) (step (a)). It was found that an intermediate (5) can be efficiently constructed by reacting the intermediate (3) with a cost-effective and commercially available 3-halo-2,4-diketone compound (4) in the presence of water (step (b)). It was also found that 2-alkylcarbonylnaphtho[2,3-b]furan-4,9-dione (6) can be constructed at a surprisingly high yield by reacting the intermediate (5) under an acidic condition (step (c)).

The present disclosure provides a method of synthesizing 2-alkylcarbonylnaphtho[2,3-b]furan-4,9-dione (6) in one step from intermediate (3) without isolating the intermediates (5) and (7) (steps (b) and (c)).

The present disclosure provides a method of synthesizing 2-alkylcarbonylnaphtho[2,3-b]furan-4,9-dione (6) in one step from 2-hydroxy-1,4-naphthoquinone (1) without isolating intermediates (3), (7), and (5) (steps (a), (b), and (c)).

In one embodiment, the present disclosure provides a special method of manufacturing 2-alkylcarbonylnaphtho[2,3-b]furan-4,9-dione (6) in only two steps from a cost-effective and commercially available raw material in a convenient, high yielding, and cost-efficient manner.

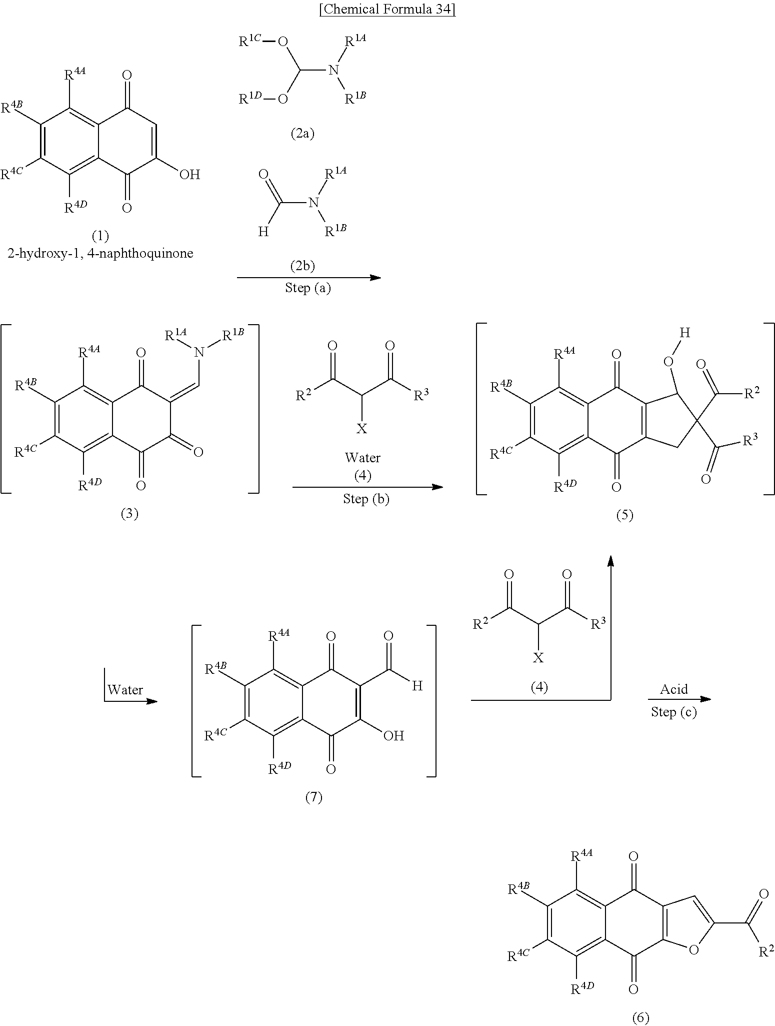

Specifically, the present invention representatively provides the following.
[Item 1] A manufacturing method of a compound or a pharmaceutically acceptable salt thereof represented by formula (6)

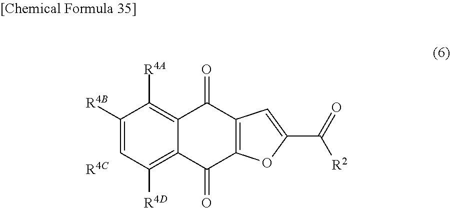

or a solvate thereof, wherein

R² is a hydrogen atom, an optionally substituted C₁₋₁₀ alkyl group, an optionally substituted C₃₋₁₀ cycloalkyl group, an optionally substituted C₆₋₁₀ aryl group, an optionally substituted 3- to 12-membered monocyclic or polycyclic heterocyclic group, and

R^{4A}, R^{4B}, R^{4C}, and R^{4D} are the same or different, each independently a hydrogen atom, a halogen atom, a cyano group, a hydroxyl group, an optionally substituted amino group, an optionally substituted C₁₋₆ alkyl group, an optionally substituted C₃₋₁₀ cycloalkyl group, an optionally substituted C₆₋₁₀ aryl group, an optionally substituted 3- to 12-membered monocyclic or polycyclic heterocyclic group, an optionally substituted C₁₋₆ alkoxy group, an optionally substituted C₃₋₁₀ cycloalkoxy group, an optionally substituted C₆₋₁₀ aryloxy group, an optionally substituted 3- to 12-membered monocyclic or polycyclic heterocyclic oxy group, a carboxyl group, an optionally substituted C₁₋₆ alkylcarbonyl group, an optionally substituted C₃₋₁₀ cycloalkylcarbonyl group, an optionally substituted C₆₋₁₀ arylcarbonyl group, an optionally substituted 3- to 12-membered monocyclic or polycyclic heterocyclic carbonyl group, an optionally substituted C₁₋₆ alkoxycarbonyl group, an optionally substituted C₃₋₁₀ cycloalkoxycarbonyl group, an optionally substituted C₆₋₁₀ aryloxycarbonyl group, an optionally substituted 3- to 12-membered monocyclic or polycyclic heterocyclic oxycarbonyl group, an optionally substituted aminocarbonyl group, an optionally substituted C₁₋₆ alkylthio group, an optionally substituted C₃₋₁₀ cycloalkylthio group, an optionally substituted C₆₋₁₀ arylthio group, an optionally substituted 3- to 12-membered monocyclic or polycyclic heterocyclic thio group, a sulfinic acid group, an optionally substituted C₁₋₆ alkylsulfinyl group, an optionally substituted C₃₋₁₀ cycloalkylsulfinyl group, an optionally substituted C₆₋₁₀ arylsulfinyl group, an optionally substituted 3- to 12-membered monocyclic or polycyclic heterocyclic sulfinyl group, an optionally substituted aminosulfinyl group, a sulfonic acid group, an optionally substituted C₁₋₆ alkylsulfonyl group, an optionally substituted C₃₋₁₀ cycloalkylsulfonyl group, an optionally substituted C₆₋₁₀ arylsulfonyl group, an optionally substituted 3- to 12-membered monocyclic or polycyclic heterocyclic sulfonyl group, or an optionally substituted aminosulfonyl group, comprising the following step (b):

(b) reacting a compound or a pharmaceutically acceptable salt thereof represented by formula (3)

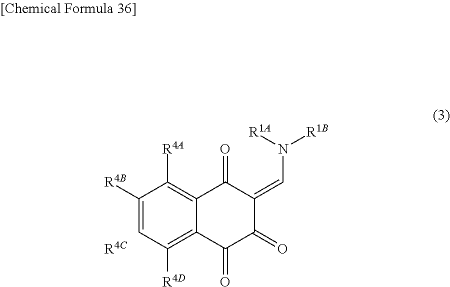

or a solvate thereof, wherein

R^{1A} and R^{1B} are the same or different, each independently a hydrogen atom, an optionally substituted C₁₋₁₀ alkyl, or an optionally substituted C₃₋₁₀ cycloalkyl group, wherein R^{1A} and R^{1B} are not simultaneously a hydrogen atom,

with a compound or a pharmaceutically acceptable salt thereof represented by formula (4)

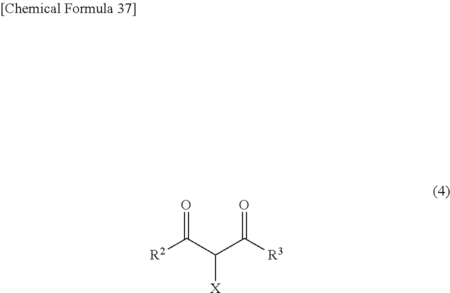

or a solvate thereof, wherein

R² is defined as the same as above,

R³ is a hydrogen atom, an optionally substituted C₁₋₁₀ alkyl group, an optionally substituted C₃₋₁₀ cycloalkyl group, an optionally substituted C₆₋₁₀ aryl group, an optionally substituted 3- to 12-membered monocyclic or polycyclic heterocyclic group, and

X is a halogen atom,

in the presence of a solvent to manufacture a compound or a pharmaceutically acceptable salt thereof represented by formula (5)

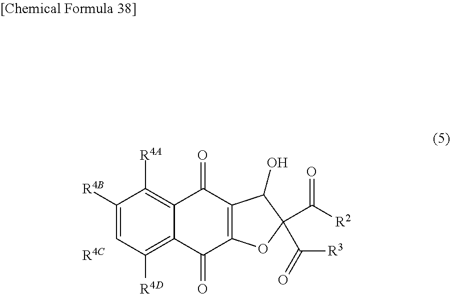

or a solvate thereof,

wherein R², R³, R^{4A}, R^{4B}, R^{4C}, and R^{4D} are defined as the same as above.
[Item 2] The manufacturing method of item 1, wherein water is added in step (b).
[Item 3] The manufacturing method of item 2, wherein an amount of water added is 1.0 equivalent to 40.0 equivalent with respect to a compound represented by formula (3) in step (b).
[Item 4] The manufacturing method of any one of items 1 to 3, wherein an amount of a compound represented by formula (4) used is 1.0 equivalent to 4.0 equivalent with respect to a compound represented by formula (3) in step (b).
[Item 5] The manufacturing method of any one of items 1 to 4, wherein the solvent is an amide-based solvent in step (b).
[Item 6] The manufacturing method of any one of items 1 to 5, wherein the solvent is an N-methyl-2-pyrrolidone in step (b).
[Item 7] The manufacturing method of any one of items 1 to 6, wherein a reaction temperature is 10° C. to 70° C. in step (b).
[Item 8] The manufacturing method of any one of items 1 to 7, further comprising the following step (c) after step (b):
   (c) heating a compound or a pharmaceutically acceptable salt thereof represented by formula (5)

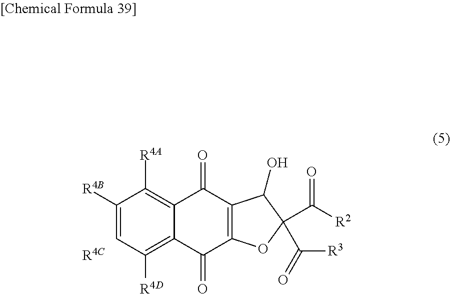

obtained in step (b) or a solvate thereof, wherein R², R³, R^{4A}, R^{4B}, R^{4C}, and R^{4D} are defined as the same as above, in the presence of a solvent to manufacture a compound or a pharmaceutically acceptable salt thereof represented by formula (6)

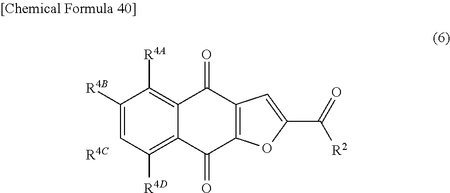

or a solvate thereof, wherein R², R^{4A}, R^{4B}, R^{4C}, and R^{4D} are defined as the same as above.
[Item 9] The manufacturing method of item 8, wherein an acid is added in step (c).
[Item 10] The manufacturing method of item 9, wherein the acid is sulfuric acid or a hydrochloric acid.
[Item 11] The manufacturing method of item 9 or 10, wherein an amount of the acid added is 1.0 equivalent to 4.0 equivalent with respect to a compound represented by formula (5) in step (c).
[Item 12] The manufacturing method of any one of items 8 to 11, wherein a reaction temperature in step (c) is 20° C. to 150° C.
[Item 13] The manufacturing method of any one of items 8 to 12, wherein a compound represented by formula (6) is deposited after completion of a reaction in step (c).
[Item 14] The manufacturing method of any one of items 8 to 13, wherein step (c) is performed without isolating the compound or pharmaceutically acceptable salt thereof represented by formula (5) or a solvate thereof in step (b)
[Item 15] The manufacturing method of any one of items 1 to 14, further comprising the following step (a) before step (b):
   (a) reacting 2-hydroxy-1,4-naphthoquinone or a pharmaceutically acceptable salt thereof represented by formula (1)

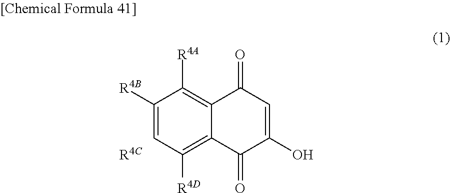

or a solvate thereof, wherein R^{4A}, R^{4B}, R^{4C}, and R^{4D} are defined as the same as above,
with a compound or a pharmaceutically acceptable salt thereof represented by formula (2a) or (2b)

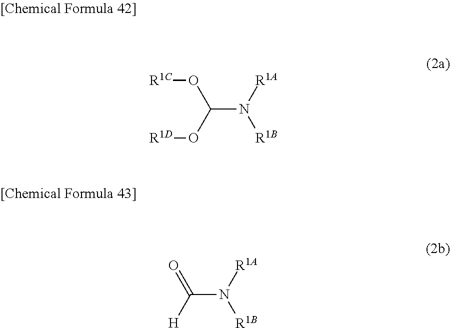

or a solvate thereof, wherein

R^{1A} and R^{1B} are defined as the same as above, and

R^{1C} and R^{1D} are the same or different, each independently a hydrogen atom, an optionally substituted C₁₋₆ alkyl group, an optionally substituted 3- to 12-membered monocyclic or polycyclic heterocyclic group, in the presence of a solvent to manufacture a compound or a pharmaceutically acceptable salt thereof represented by formula (3)

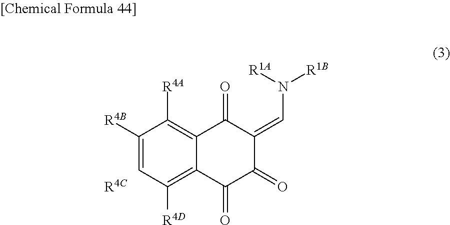

or a solvate thereof, wherein R^{1A}, R^{1B}, R^{4A}, R^{4B}, R^{4C}, and R^{4D} are defined as the same as above.
[Item 16] The manufacturing method of item 15, wherein the solvent is an amide-based solvent in step (a).
[Item 17] The manufacturing method of item 15 or 16, wherein the solvent is N,N-dimethylformamide or N-methyl-2-pyrrolidone in step (a).
[Item 18] The manufacturing method of any one of items 15 to 17, wherein a reaction temperature is −10° C. to 30° C. in step (a).
[Item 19] The manufacturing method of any one of items 15 to 18, wherein an amount of a compound represented by formula (2a) or (2b) used is 1.0 equivalent to 10.0 equivalent with respect to a compound represented by formula (1) in step (a).
[Item 20] The manufacturing method of any one of items 15 to 19, wherein a compound represented by formula (2a) is used in step (a).
[Item 21] The manufacturing method of any one of items 15 to 20, wherein a compound represented by formula (3) is deposited after completion of a reaction in step (a).
[Item 22] The manufacturing method of item 21, wherein a solid that is the deposited compound represented by formula (3) is filtered out and washed with an alcohol-based solvent in step (a).
[Item 23] The manufacturing method of item 22, wherein the alcohol-based solvent is methanol.
[Item 24] The manufacturing method of any one of items 15 to 23, wherein steps (a), (b), and (c) are performed without isolating the compounds represented by formula (3) and formula (5) in steps (a), (b), and (c).
[Item 25] The manufacturing method of any one of items 1 to 24, wherein R^{1A} and R^{1B} are methyl groups.
[Item 26] The manufacturing method of any one of items 15 to 25, wherein R^{1C} and R^{1D} are methyl groups.
[Item 27] The manufacturing method of any one of items 1 to 26, wherein R² is a methyl group.
[Item 28] The manufacturing method of any one of items 1 to 27, wherein R³ is a methyl group.
[Item 29] The manufacturing method of any one of items 1 to 28, wherein R^{4A}, R^{4B}, R^{4C}, and R^{4D} are hydrogen atoms.
[Item 30] A manufacturing method of a compound or a pharmaceutically acceptable salt thereof represented by formula (3)

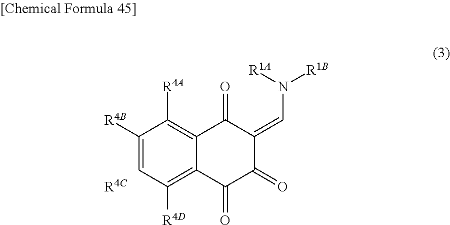

or a solvate thereof, wherein

R^{1A} and R^{1B} are the same or different, each independently a hydrogen atom, an optionally substituted C₁₋₁₀ alkyl group, or an optionally substituted C₃₋₁₀ cycloalkyl group, wherein R^{1A} and R^{1B} are not simultaneously a hydrogen atom, and

R^{4A}, R^{4B}, R^{4C}, and R^{4D} are the same or different, each independently a hydrogen atom, a halogen atom, a cyano group, a hydroxyl group, an optionally substituted amino group, an optionally substituted C₁₋₆ alkyl group, an optionally substituted C₃₋₁₀ cycloalkyl group, an optionally substituted C₆₋₁₀ aryl group, an optionally substituted 3- to 12-membered monocyclic or polycyclic heterocyclic group, an optionally substituted C₁₋₆ alkoxy group, an optionally substituted C₃₋₁₀ cycloalkoxy group, an optionally substituted C₆₋₁₀ aryloxy group, an optionally substituted 3- to 12-membered monocyclic or polycyclic heterocyclic oxy group, a carboxyl group, an optionally substituted C₁₋₆ alkylcarbonyl group, an optionally substituted C₃₋₁₀ cycloalkylcarbonyl group, an optionally substituted C₆₋₁₀ arylcarbonyl group, an optionally substituted 3- to 12-membered monocyclic or polycyclic heterocyclic carbonyl group, an optionally substituted C₁₋₆ alkoxycarbonyl group, an optionally substituted C₃₋₁₀ cycloalkoxycarbonyl group, an optionally substituted C₆₋₁₀ aryloxycarbonyl group, an optionally substituted 3- to 12-membered monocyclic or polycyclic heterocyclic oxycarbonyl group, an optionally substituted aminocarbonyl group, an optionally substituted C₁₋₆ alkylthio group, an optionally substituted C₃₋₁₀ cycloalkylthio group, an optionally substituted C₆₋₁₀ arylthio group, an optionally substituted 3- to 12-membered monocyclic or polycyclic heterocyclic thio group, a sulfinic acid group, an optionally substituted C₁₋₆ alkylsulfinyl group, an optionally substituted C₃₋₁₀ cycloalkylsulfinyl group, an optionally substituted C₆₋₁₀ arylsulfinyl group, an optionally substituted 3- to 12-membered monocyclic or polycyclic heterocyclic sulfinyl group, an optionally substituted aminosulfinyl group, a sulfonic acid group, an optionally substituted C₁₋₆ alkylsulfonyl group, an optionally substituted C₃₋₁₀ cycloalkylsulfonyl group, an optionally substituted C₆₋₁₀ arylsulfonyl group, an optionally substituted 3- to 12-membered monocyclic or polycyclic heterocyclic sulfonyl group, or an optionally substituted aminosulfonyl group, comprising the following step (a):

(a) reacting 2-hydroxy-1,4-naphthoquinone or a pharmaceutically acceptable salt thereof represented by formula (1)

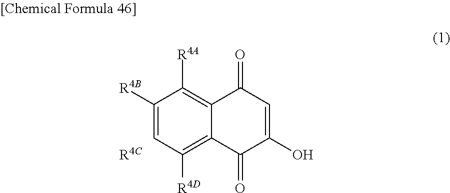

or a solvate thereof, wherein R^{4A}, R^{4B}, R^{4C}, and R^{4D} are defined as the same as above,
with a compound or a pharmaceutically acceptable salt thereof represented by formula (2a) or (2b)

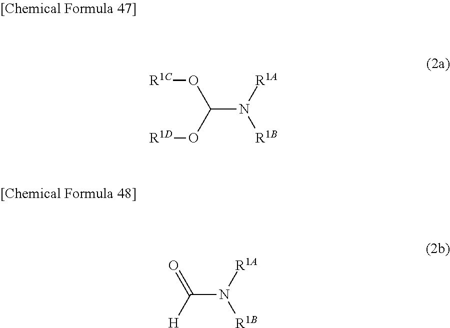

or a solvate thereof, wherein

R^{1A} and R^{1B} are defined as the same as above, and

R^{1C} and R^{1D} are the same or different, each independently a hydrogen atom, an optionally substituted C₁₋₆ alkyl group, an optionally substituted 3- to 12-membered monocyclic or polycyclic heterocyclic group, in the presence of a solvent to manufacture a compound or a pharmaceutically acceptable salt thereof represented by formula (3)

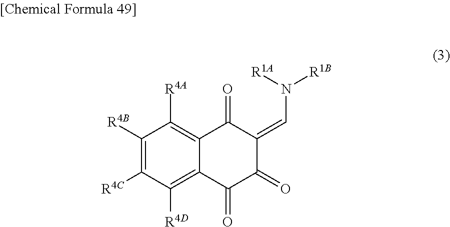

or a solvate thereof,
wherein R^{1A}, R^{1B}, R^{4A}, R^{4B}, R^{4C}, and R^{4D} are defined as the same as above.
[Item 31] The manufacturing method of item 30, wherein the solvent is an amide-based solvent in step (a).
[Item 32] The manufacturing method of item 30 or 31, wherein the solvent is N,N-dimethylformamide or N-methyl-2-pyrrolidone in step (a).
[Item 33] The manufacturing method of any one of items 30 to 32, wherein a reaction temperature is −10° C. to 30° C. in step (a).
[Item 34] The manufacturing method of any one of items 30 to 33, wherein an amount of a compound represented by formula (2a) or (2b) used is 1.0 equivalent to 10.0 equivalent with respect to a compound represented by formula (1) in step (a).
[Item 35] The manufacturing method of any one of items 30 to 34, wherein a compound represented by formula (2a) is used in step (a).
[Item 36] The manufacturing method of any one of items 30 to 35, wherein a compound represented by formula (3) is deposited after completion of a reaction in step (a).
[Item 37] The manufacturing method of item 36, wherein a solid that is the deposited compound represented by formula (3) is filtered out and washed with an alcohol-based solvent in step (a).
[Item 38] The manufacturing method of item 37, wherein the alcohol-based solvent is methanol.
[Item 39] The manufacturing method of any one of items 30 to 38, wherein R^{1A} and R^{1B} are methyl groups.
[Item 40] The manufacturing method of any one of items 30 to 39, wherein R^{1C} and R^{4D} are methyl groups.
[Item 41] The manufacturing method of any one of items 30 to 40, wherein R^{4A}, R^{4B}, R^{4C}, and R^{4D} are hydrogen atoms.
[Item 42] A manufacturing method of a compound or a pharmaceutically acceptable salt thereof represented by formula (5)

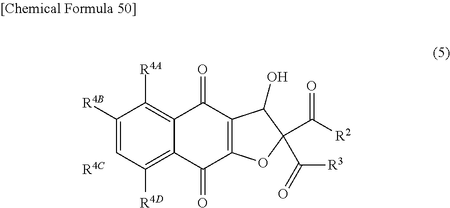

or a solvate thereof, wherein

R² is a hydrogen atom, an optionally substituted C₁₋₁₀ alkyl group, an optionally substituted C₃₋₁₀ cycloalkyl group, an optionally substituted C₆₋₁₀ aryl group, or an optionally substituted 3- to 12-membered monocyclic or polycyclic heterocyclic group,

R³ is a hydrogen atom, an optionally substituted C₁₋₁₀ alkyl group, an optionally substituted C₃₋₁₀ cycloalkyl group, an optionally substituted C₆₋₁₀ aryl group, or an optionally substituted 3- to 12-membered monocyclic or polycyclic heterocyclic group, and

R^{4A}, R^{4B}, R^{4C}, and R^{4D} are the same or different, each independently a hydrogen atom, a halogen atom, a cyano group, a hydroxyl group, an optionally substituted amino group, an optionally substituted C₁₋₆ alkyl group, an optionally substituted C₃₋₁₀ cycloalkyl group, an optionally substituted C₆₋₁₀ aryl group, an optionally substituted 3- to 12-membered monocyclic or polycyclic heterocyclic group, an optionally substituted C₁₋₆ alkoxy group, an optionally substituted C₃₋₁₀ cycloalkoxy group, an optionally substituted C₆₋₁₀ aryloxy group, an optionally substituted 3- to 12-membered monocyclic or polycyclic heterocyclic oxy group, a carboxyl group, an optionally substituted C₁₋₆ alkylcarbonyl group, an optionally substituted C₃₋₁₀ cycloalkylcarbonyl group, an optionally substituted C₆₋₁₀ arylcarbonyl group, an optionally substituted 3- to 12-membered monocyclic or polycyclic heterocyclic carbonyl group, an optionally substituted C₁₋₆ alkoxycarbonyl group, an optionally substituted C₃₋₁₀ cycloalkoxycarbonyl group, an optionally substituted C₆₋₁₀ aryloxycarbonyl group, an optionally substituted 3- to 12-membered monocyclic or polycyclic heterocyclic oxycarbonyl group, an optionally substituted aminocarbonyl group, an optionally substituted C₁₋₆ alkylthio group, an optionally substituted C₃₋₁₀ cycloalkylthio group, an optionally substituted C₆₋₁₀ arylthio group, an optionally substituted 3- to 12-membered monocyclic or polycyclic heterocyclic thio group, a sulfinic acid group, an optionally substituted C₁₋₆ alkylsulfinyl group, an optionally substituted C₃₋₁₀ cycloalkylsulfinyl group, an optionally substituted C₆₋₁₀ arylsulfinyl group, an optionally substituted 3- to 12-membered monocyclic or polycyclic heterocyclic sulfinyl group, an optionally substituted aminosulfinyl group, a sulfonic acid group, an optionally substituted C₁₋₆ alkylsulfonyl group, an optionally substituted C₃₋₁₀ cycloalkylsulfonyl group, an optionally substituted C₆₋₁₀ arylsulfonyl group, an optionally substituted 3- to 12-membered monocyclic or polycyclic heterocyclic sulfonyl group, or an optionally substituted aminosulfonyl group, comprising the following step (b):

(b) reacting a compound or a pharmaceutically acceptable salt thereof represented by formula (3)

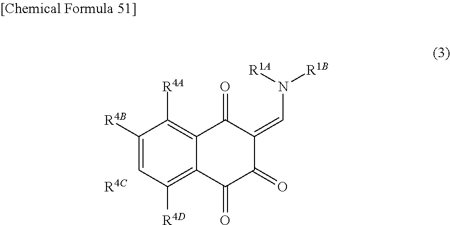

or a solvate thereof,
wherein R^{1A} and R^{1B} are the same or different, each independently a hydrogen atom, an optionally substituted C₁₋₁₀ alkyl, or an optionally substituted C₃₋₁₀ cycloalkyl group, wherein R^{1A} and R^{1B} are not simultaneously a hydrogen atom,
with a compound or a pharmaceutically acceptable salt thereof represented by formula (4)

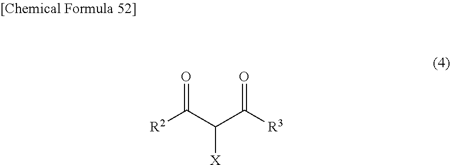

or a solvate thereof, wherein R² and R³ are defined as the same as above, and X is a halogen atom,
in the presence of a solvent to manufacture a compound or a pharmaceutically acceptable salt thereof represented by formula (5)

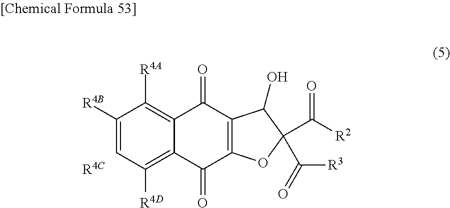

or a solvate thereof,
wherein R², R³, R^{4A}, R^{4B}, R^{4C}, and R^{4D} are defined as the same as above.
[Item 43] The manufacturing method of item 42, wherein water is added in step (b).
[Item 44] The manufacturing method of item 43, wherein an amount of water added is 1.0 equivalent to 40.0 equivalent with respect to a compound represented by formula (3) in step (b).
[Item 45] The manufacturing method of any one of items 42 to 44, wherein an amount of a compound represented by formula (4) used is 1.0 equivalent to 4.0 equivalent with respect to a compound represented by formula (3) in step (b).
[Item 46] The manufacturing method of any one of items 42 to 45, wherein the solvent is an amide-based solvent in step (b).
[Item 47] The manufacturing method of any one of items 42 to 46, wherein the solvent is an N-methyl-2-pyrrolidone in step (b).
[Item 48] The manufacturing method of any one of items 42 to 47, wherein a reaction temperature is 10° C. to 70° C. in step (b).
[Item 49] The manufacturing method of any one of items 42 to 48, further comprising the following step (a) before step (b):
   (a) reacting 2-hydroxy-1,4-naphthoquinone or a pharmaceutically acceptable salt thereof represented by formula (1)

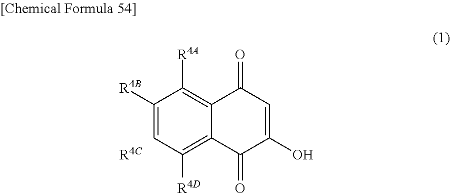

or a solvate thereof, wherein R^{4A}, R^{4B}, R^{4C}, and R^{4D} are defined as the same as above,
with a compound or a pharmaceutically acceptable salt thereof represented by formula (2a) or (2b)

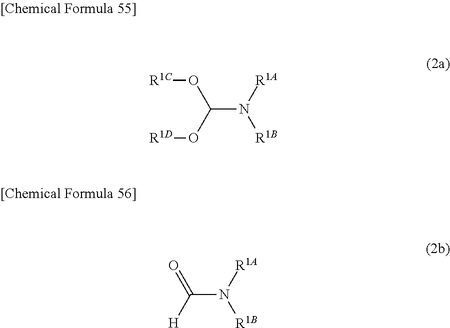

or a solvate thereof, wherein R^{1A} and R^{1B} are defined as the same as above, and R^{1C} and R^{1D} are the same or different, each independently a hydrogen atom, an optionally substituted C₁₋₆ alkyl group, or an optionally substituted 3- to 12-membered monocyclic or polycyclic heterocyclic group, in the presence of a solvent to manufacture a compound or a pharmaceutically acceptable salt thereof represented by formula (3)

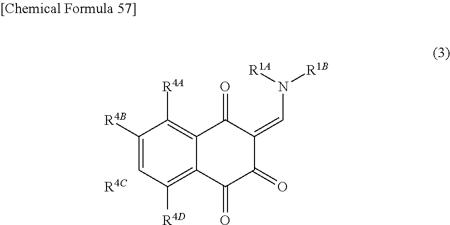

or a solvate thereof, wherein R^{1A}, R^{1B}, R^{4A}, R^{4B}, R^{4C}, and R^{4D} are defined as the same as above.
[Item 50] The manufacturing method of item 49, wherein the solvent is an amide-based solvent in step (a).
[Item 51] The manufacturing method of item 49 or 50, wherein the solvent is N,N-dimethylformamide or N-methyl-2-pyrrolidone in step (a).
[Item 52] The manufacturing method of any one of items 49 to 51, wherein a reaction temperature is −10° C. to 30° C. in step (a).
[Item 53] The manufacturing method of any one of items 49 to 52, wherein an amount of a compound represented by formula (2a) or (2b) used is 1.0 equivalent to 10.0 equivalent with respect to a compound represented by formula (1) in step (a).
[Item 54] The manufacturing method of any one of items 49 to 53, wherein a compound represented by formula (2a) is used in step (a).
[Item 55] The manufacturing method of any one of items 49 to 54, wherein a compound represented by formula (3) is deposited after completion of a reaction in step (a).
[Item 56] The manufacturing method of item 55, wherein a solid that is the deposited compound represented by formula (3) is filtered out and washed with an alcohol-based solvent in step (a).
[Item 57] The manufacturing method of item 56, wherein the alcohol-based solvent is methanol.
[Item 58] The manufacturing method of any one of items 42 to 57, wherein R^{1A} and R^{1B} are methyl groups.
[Item 59] The manufacturing method of any one of items 49 to 58, wherein R^{1C} and R^{4D} are methyl groups.
[Item 60] The manufacturing method of any one of items 42 to 59, wherein R² is a methyl group.
[Item 61] The manufacturing method of any one of items 42 to 60, wherein R³ is a methyl group.
[Item 62] The manufacturing method of any one of items 42 to 61, wherein R^{4A}, R^{4B}, R^{4C}, and R^{4D} are hydrogen atoms.
[Item 63] A manufacturing method of a compound or a pharmaceutically acceptable salt thereof represented by formula (6)

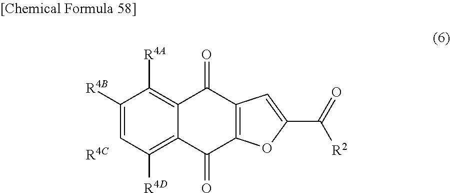

or a solvate thereof, wherein

R² is a hydrogen atom, an optionally substituted C₁₋₁₀ alkyl group, an optionally substituted C₃₋₁₀ cycloalkyl group, an optionally substituted C₆₋₁₀ aryl group, or an optionally substituted 3- to 12-membered monocyclic or polycyclic heterocyclic group, and

R^{4A}, R^{4B}, R^{4C}, and R^{4D} are the same or different, each independently a hydrogen atom, a halogen atom, a cyano group, a hydroxyl group, an optionally substituted amino group, an optionally substituted C₁₋₆ alkyl group, an optionally substituted C₃₋₁₀ cycloalkyl group, an optionally substituted C₆₋₁₀ aryl group, an optionally substituted 3- to 12-membered monocyclic or polycyclic heterocyclic group, an optionally substituted C₁₋₆ alkoxy group, an optionally substituted C₃₋₁₀ cycloalkoxy group, an optionally substituted C₆₋₁₀ aryloxy group, an optionally substituted 3- to 12-membered monocyclic or polycyclic heterocyclic oxy group, a carboxyl group, an optionally substituted C₁₋₆ alkylcarbonyl group, an optionally substituted C₃₋₁₀ cycloalkylcarbonyl group, an optionally substituted C₆₋₁₀ arylcarbonyl group, an optionally substituted 3- to 12-membered monocyclic or polycyclic heterocyclic carbonyl group, an optionally substituted C₁₋₆ alkoxycarbonyl group, an optionally substituted C₃₋₁₀ cycloalkoxycarbonyl group, an optionally substituted C₆₋₁₀ aryloxycarbonyl group, an optionally substituted 3- to 12-membered monocyclic or polycyclic heterocyclic oxycarbonyl group, an optionally substituted aminocarbonyl group, an optionally substituted C₁₋₆ alkylthio group, an optionally substituted C₃₋₁₀ cycloalkylthio group, an optionally substituted C₆₋₁₀ arylthio group, an optionally substituted 3- to 12-membered monocyclic or polycyclic heterocyclic thio group, a sulfinic acid group, an optionally substituted C₁₋₆ alkylsulfinyl group, an optionally substituted C₃₋₁₀ cycloalkylsulfinyl group, an optionally substituted C₆₋₁₀ arylsulfinyl group, an optionally substituted 3- to 12-membered monocyclic or polycyclic heterocyclic sulfinyl group, an optionally substituted aminosulfinyl group, a sulfonic acid group, an optionally substituted C₁₋₆ alkylsulfonyl group, an optionally substituted C₃₋₁₀ cycloalkylsulfonyl group, an optionally substituted C₆₋₁₀ arylsulfonyl group, an optionally substituted 3- to 12-membered monocyclic or polycyclic heterocyclic sulfonyl group, or an optionally substituted aminosulfonyl group, comprising the following step (e):

(e) reacting a compound or a pharmaceutically acceptable salt thereof represented by formula (8)

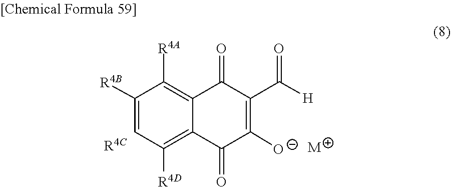

or a solvate thereof, wherein

M is Li, Na, or K, and

R^{4A}, R^{4B}, R^{4C}, and R^{4D} are defined as the same as above, with a compound or a pharmaceutically acceptable salt thereof represented by formula (4)

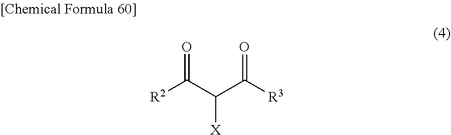

or a solvate thereof,
wherein

R² is defined as the same as above,

R³ is a hydrogen atom, an optionally substituted C₁₋₁₀ alkyl group, an optionally substituted C₃₋₁₀ cycloalkyl group, an optionally substituted C₆₋₁₀ aryl group, an optionally substituted 3- to 12-membered monocyclic or polycyclic heterocyclic group, and

X is a halogen atom

in the presence of a solvent to manufacture a compound or a pharmaceutically acceptable salt thereof represented by formula (5)

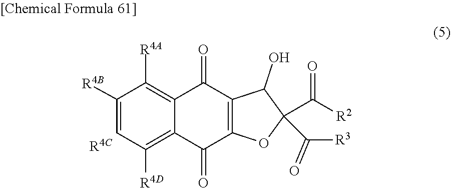

or a solvate thereof,
wherein R², R³, R^{4A}, R^{4B}, R^{4C}, and R^{4D} are defined as the same as above.
[Item 64] The manufacturing method of item 63, wherein the solvent is an amide-based solvent in step (e).
[Item 65] The manufacturing method of item 63 or 64, wherein the solvent in step (e) is an N-methyl-2-pyrrolidone.
[Item 66] The manufacturing method of any one of items 63 to 65, wherein an amount of a compound represented by formula (4) used is 1.0 equivalent to 4.0 equivalent with respect to a compound represented by formula (8) in step (e).
[Item 67] The manufacturing method of any one of items 63 to 66, wherein a reaction temperature is 10° C. to 70° C. in step (e).
[Item 68] The manufacturing method of any one of items 63 to 67, further comprising the following step (c) after step (e):
   (c) heating a compound or a pharmaceutically acceptable salt thereof represented by formula (5)

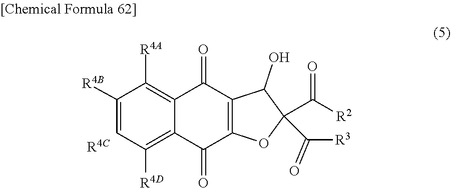

obtained in step (e), or a solvate thereof, wherein R², R³, R^{4A}, R^{4B}, R^{4C}, and R^{4D} are defined as the same as above, in the presence of a solvent to manufacture a compound or a pharmaceutically acceptable salt thereof represented by formula (6)

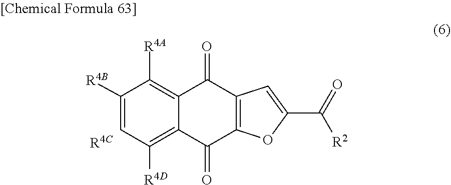

or a solvate thereof, wherein R² is defined as the same as above.
[Item 69] The manufacturing method of item 68, wherein an acid is added in step (c).
[Item 70] The manufacturing method of item 69, wherein the acid is sulfuric acid or a hydrochloric acid.
[Item 71] The manufacturing method of item 69 or 70, wherein an amount of the acid added is 1.0 equivalent to 4.0 equivalent with respect to a compound represented by formula (5) in step (c).
[Item 72] The manufacturing method of any one of items 68 to 71, wherein a reaction temperature in step (c) is 20° C. to 150° C.
[Item 73] The manufacturing method of any one of items 68 to 72, wherein a compound represented by formula (6) is deposited after completion of a reaction in step (c).
[Item 74] The manufacturing method of any one of items 68 to 73, wherein step (c) is performed without isolating the compound or pharmaceutically acceptable salt thereof represented by formula (5) or a solvate thereof in step (e)
[Item 75] The manufacturing method of any one of items 68 to 74, further comprising the following step (d) before step (e):
   (d) reacting a compound or a pharmaceutically acceptable salt thereof represented by formula (3)

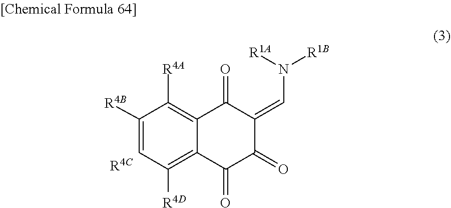

or a solvate thereof, wherein

R^{1A} and R^{1B} are the same or different, each independently a hydrogen atom, an optionally substituted C₁₋₁₀ alkyl, or an optionally substituted C₃₋₁₀ cycloalkyl group, wherein R^{1A} and R^{1B} are not simultaneously a hydrogen atom, and

R^{4A}, R^{4B}, R^{4C}, and R^{4D} are defined as the same as above, with a base in a solvent to manufacture a compound or a pharmaceutically acceptable salt thereof represented by formula (8)

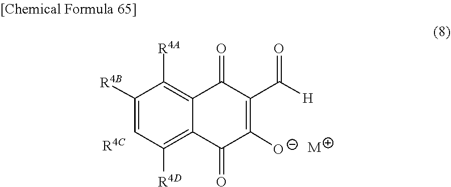

or a solvate thereof, wherein M, R^{4A}, R^{4B}, R^{4C}, and R^{4D} are defined as the same as above.
[Item 76] The manufacturing method of item 75, wherein a reaction is performed in the presence of water in step (d).
[Item 77] The manufacturing method of item 76, wherein an amount of water used is 0.1-fold to 10.0-fold in weight with respect to a compound represented by formula (3) in step (d).
[Item 78] The manufacturing method of any one of items 75 to 77, wherein the base is an inorganic base in step (d).
[Item 79] The manufacturing method of item 78, wherein the inorganic base is potassium carbonate.
[Item 80] The manufacturing method of any one of items 75 to 79, wherein an amount of a base used is 1.0 equivalent to 10.0 equivalent with respect to a compound represented by formula (3) in step (d).
[Item 81] The manufacturing method of any one of items 75 to 80, wherein the solvent is an amide-based solvent in step (d).
[Item 82] The manufacturing method of any one of items 75 to 81, wherein the solvent is N,N-dimethylformamide or N-methyl-2-pyrrolidone in step (d).
[Item 83] The manufacturing method of any one of items 75 to 82, wherein a compound represented by formula (8) is deposited after completion of a reaction in step (d).
[Item 84] The manufacturing method of any one of items 75 to 83, wherein a reaction temperature is 0° C. to 50° C. in step (d).
[Item 85] The manufacturing method of any one of items 63 to 84, wherein R^{1A} and R^{1B} are methyl groups.
[Item 86] The manufacturing method of any one of items 63 to 85, wherein R² is a methyl group.
[Item 87] The manufacturing method of any one of items 63 to 86, wherein R³ is a methyl group.
[Item 88] The manufacturing method of any one of items 63 to 87, wherein R^{4A}, R^{4B}, R^{4C}, and R⁴ are hydrogen atoms.
[Item 89] The manufacturing method of any one of items 63 to 88, wherein M is potassium.
[Item 90] A compound or a pharmaceutically acceptable salt thereof represented by formula (3)

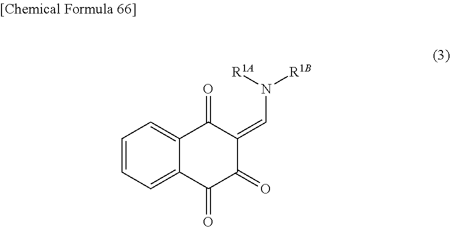

or a solvate thereof, wherein

R^{1A} and R^{1B} are the same or different, each independently a hydrogen atom, an optionally substituted C₁₋₁₀ alkyl, or an optionally substituted C₃₋₁₀ cycloalkyl group, wherein R^{1A} and R^{1B} are not simultaneously a hydrogen atom.
[Item 91] The compound or a pharmaceutically acceptable salt, or a solvate thereof of item 90, wherein R^{1A} and R^{1B} are optionally substituted C₁₋₁₀ alkyl groups.
[Item 92] The compound or a pharmaceutically acceptable salt, or a solvate thereof of item 90 or 91, wherein R^{1A} and R^{1B} are methyl groups.
[Item 93] A compound or a pharmaceutically acceptable salt thereof represented by formula (5)

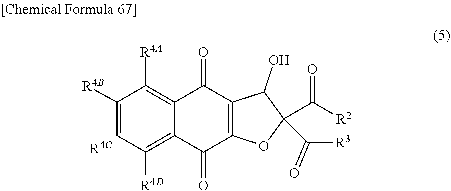

or a solvate thereof, wherein

R² is a hydrogen atom, an optionally substituted C₁₋₁₀ alkyl group, an optionally substituted C₃₋₁₀ cycloalkyl group, an optionally substituted C₆₋₁₀ aryl group, or an optionally substituted 3- to 12-membered monocyclic or polycyclic heterocyclic group,

R³ is a hydrogen atom, an optionally substituted C₁₋₁₀ alkyl group, an optionally substituted C₃₋₁₀ cycloalkyl group, an optionally substituted C₆₋₁₀ aryl group, or an optionally substituted 3- to 12-membered monocyclic or polycyclic heterocyclic group, and R^{4A}, R^{4B}, R^{4C}, and R^{4D} are the same or different, each independently a hydrogen atom, a halogen atom, a cyano group, a hydroxyl group, an optionally substituted amino group, an optionally substituted C₁₋₆ alkyl group, an optionally substituted C₃₋₁₀ cycloalkyl group, an optionally substituted C₆₋₁₀ aryl group, an optionally substituted 3- to 12-membered monocyclic or polycyclic heterocyclic group, an optionally substituted C₁₋₆ alkoxy group, an optionally substituted C₃₋₁₀ cycloalkoxy group, an optionally substituted C₆₋₁₀ aryloxy group, an optionally substituted 3- to 12-membered monocyclic or polycyclic heterocyclic oxy group, a carboxyl group, an optionally substituted C₁₋₆ alkylcarbonyl group, an optionally substituted C₃₋₁₀ cycloalkylcarbonyl group, an optionally substituted C₆₋₁₀ arylcarbonyl group, an optionally substituted 3- to 12-membered monocyclic or polycyclic heterocyclic carbonyl group, an optionally substituted C₁₋₆ alkoxycarbonyl group, an optionally substituted C₃₋₁₀ cycloalkoxycarbonyl group, an optionally substituted C₆₋₁₀ aryloxycarbonyl group, an optionally substituted 3- to 12-membered monocyclic or polycyclic heterocyclic oxycarbonyl group, an optionally substituted aminocarbonyl group, an optionally substituted C₁₋₆ alkylthio group, an optionally substituted C₃₋₁₀ cycloalkylthio group, an optionally substituted C₆₋₁₀ arylthio group, an optionally substituted 3- to 12-membered monocyclic or polycyclic heterocyclic thio group, a sulfinic acid group, an optionally substituted C₁₋₆ alkylsulfinyl group, an optionally substituted C₃₋₁₀ cycloalkylsulfinyl group, an optionally substituted C₆₋₁₀ arylsulfinyl group, an optionally substituted 3- to 12-membered monocyclic or polycyclic heterocyclic sulfinyl group, an optionally substituted aminosulfinyl group, a sulfonic acid group, an optionally substituted C₁₋₆ alkylsulfonyl group, an optionally substituted C₃₋₁₀ cycloalkylsulfonyl group, an optionally substituted C₆₋₁₀ arylsulfonyl group, an optionally substituted 3- to 12-membered monocyclic or polycyclic heterocyclic sulfonyl group, or an optionally substituted aminosulfonyl group.
[Item 94] The compound or a pharmaceutically acceptable salt, or a solvate thereof of item 93, wherein R² is an optionally substituted C₁₋₁₀ alkyl group.
[Item 95] The compound or a pharmaceutically acceptable salt, or a solvate thereof of item 93 or 94, wherein R² is a methyl group.
[Item 96] The compound or a pharmaceutically acceptable salt, or a solvate thereof of any one of items 93 to 95, wherein R³ is an optionally substituted C₁₋₁₀ alkyl group.
[Item 97] The compound or a pharmaceutically acceptable salt, or a solvate thereof of any one of items 93 to 96, wherein R³ is a methyl group.
[Item 98] The compound or a pharmaceutically acceptable salt, or a solvate thereof of any one of items 93 to 97, wherein R^{4A}, R^{4B}, R^{4C}, and R^{4D} are the same or different, each independently selected from the group consisting of a hydrogen atom, a halogen atom, a cyano group, a hydroxyl group, an optionally substituted amino group, and an optionally substituted C₁₋₆ alkyl group.
[Item 99] The compound or a pharmaceutically acceptable salt, or a solvate thereof of any one of items 93 to 98, wherein R^{4A}, R^{4B}, R^{4C}, and R^{4D} are hydrogen atoms. [Item 100] A compound or a pharmaceutically acceptable salt thereof represented by formula (7)

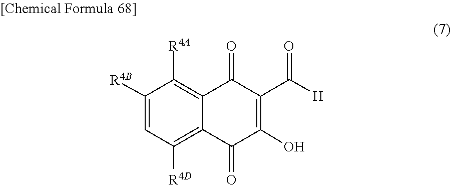

or a solvate thereof, wherein

R^{4A}, R^{4B}, R^{4C}, and R^{4D} are the same or different, each independently a hydrogen atom, a halogen atom, a cyano group, a hydroxyl group, an optionally substituted amino group, an optionally substituted C₁₋₆ alkyl group, an optionally substituted C₃₋₁₀ cycloalkyl group, an optionally substituted C₆₋₁₀ aryl group, an optionally substituted 3- to 12-membered monocyclic or polycyclic heterocyclic group, an optionally substituted C₁₋₆ alkoxy group, an optionally substituted C₃₋₁₀ cycloalkoxy group, an optionally substituted C₆₋₁₀ aryloxy group, an optionally substituted 3- to 12-membered monocyclic or polycyclic heterocyclic oxy group, a carboxyl group, an optionally substituted C₁₋₆ alkylcarbonyl group, an optionally substituted C₃₋₁₀ cycloalkylcarbonyl group, an optionally substituted C₆₋₁₀ arylcarbonyl group, an optionally substituted 3- to 12-membered monocyclic or polycyclic heterocyclic carbonyl group, an optionally substituted C₁₋₆ alkoxycarbonyl group, an optionally substituted C₃₋₁₀ cycloalkoxycarbonyl group, an optionally substituted C₆₋₁₀ aryloxycarbonyl group, an optionally substituted 3- to 12-membered monocyclic or polycyclic heterocyclic oxycarbonyl group, an optionally substituted aminocarbonyl group, an optionally substituted C₁₋₆ alkylthio group, an optionally substituted C₃₋₁₀ cycloalkylthio group, an optionally substituted C₆₋₁₀ arylthio group, an optionally substituted 3- to 12-membered monocyclic or polycyclic heterocyclic thio group, a sulfinic acid group, an optionally substituted C₁₋₆ alkylsulfinyl group, an optionally substituted C₃₋₁₀ cycloalkylsulfinyl group, an optionally substituted C₆₋₁₀ arylsulfinyl group, an optionally substituted 3- to 12-membered monocyclic or polycyclic heterocyclic sulfinyl group, an optionally substituted aminosulfinyl group, a sulfonic acid group, an optionally substituted C₁₋₆ alkylsulfonyl group, an optionally substituted C₃₋₁₀ cycloalkylsulfonyl group, an optionally substituted C₆₋₁₀ arylsulfonyl group, an optionally substituted 3- to 12-membered monocyclic or polycyclic heterocyclic sulfonyl group, or an optionally substituted aminosulfonyl group.
[Item 101]

A compound or a pharmaceutically acceptable salt thereof represented by formula (8)

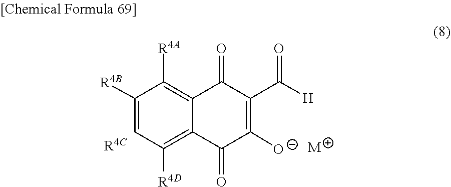

or a solvate thereof, wherein

M is Li, Na, or K, and

R^{4A}, R^{4B}, R^{4C}, and R^{4D} are the same or different, each independently a hydrogen atom, a halogen atom, a cyano group, a hydroxyl group, an optionally substituted amino group, an optionally substituted C₁₋₆ alkyl group, an optionally substituted C₃₋₁₀ cycloalkyl group, an optionally substituted C₆₋₁₀ aryl group, an optionally substituted 3- to 12-membered monocyclic or polycyclic heterocyclic group, an optionally substituted C₁₋₆ alkoxy group, an optionally substituted C₃₋₁₀ cycloalkoxy group, an optionally substituted C₆₋₁₀ aryloxy group, an optionally substituted 3- to 12-membered monocyclic or polycyclic heterocyclic oxy group, a carboxyl group, an optionally substituted C₁₋₆ alkylcarbonyl group, an optionally substituted C₃₋₁₀ cycloalkylcarbonyl group, an optionally substituted C₆₋₁₀ arylcarbonyl group, an optionally substituted 3- to 12-membered monocyclic or polycyclic heterocyclic carbonyl group, an optionally substituted C₁₋₆ alkoxycarbonyl group, an optionally substituted C₃₋₁₀ cycloalkoxycarbonyl group, an optionally substituted C₆₋₁₀ aryloxycarbonyl group, an optionally substituted 3- to 12-membered monocyclic or polycyclic heterocyclic oxycarbonyl group, an optionally substituted aminocarbonyl group, an optionally substituted C₁₋₆ alkylthio group, an optionally substituted C₃₋₁₀ cycloalkylthio group, an optionally substituted C₆₋₁₀ arylthio group, an optionally substituted 3- to 12-membered monocyclic or polycyclic heterocyclic thio group, a sulfinic acid group, an optionally substituted C₁₋₆ alkylsulfinyl group, an optionally substituted C₃₋₁₀ cycloalkylsulfinyl group, an optionally substituted C₆₋₁₀ arylsulfinyl group, an optionally substituted 3- to 12-membered monocyclic or polycyclic heterocyclic sulfinyl group, an optionally substituted aminosulfinyl group, a sulfonic acid group, an optionally substituted C₁₋₆ alkylsulfonyl group, an optionally substituted C₃₋₁₀ cycloalkylsulfonyl group, an optionally substituted C₆₋₁₀ arylsulfonyl group, an optionally substituted 3- to 12-membered monocyclic or polycyclic heterocyclic sulfonyl group, or an optionally substituted aminosulfonyl group. [Item 102]A manufacturing method of a compound or a pharmaceutically acceptable salt thereof represented by formula (6)

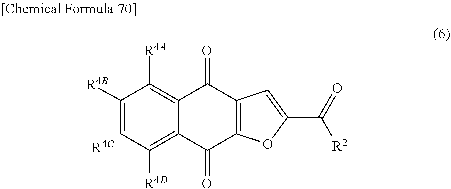

or a solvate thereof, wherein

R² is a hydrogen atom, an optionally substituted C₁₋₁₀ alkyl group, an optionally substituted C₃₋₁₀ cycloalkyl group, an optionally substituted C₆₋₁₀ aryl group, an optionally substituted 3- to 12-membered monocyclic or polycyclic heterocyclic group, and

R^{4A}, R^{4B}, R^{4C}, and R^{4D} are the same or different, each independently a hydrogen atom, a halogen atom, a cyano group, a hydroxyl group, an optionally substituted amino group, an optionally substituted C₁₋₆ alkyl group, an optionally substituted C₃₋₁₀ cycloalkyl group, an optionally substituted C₆₋₁₀ aryl group, an optionally substituted 3- to 12-membered monocyclic or polycyclic heterocyclic group, an optionally substituted C₁₋₆ alkoxy group, an optionally substituted C₃₋₁₀ cycloalkoxy group, an optionally substituted C₆₋₁₀ aryloxy group, an optionally substituted 3- to 12-membered monocyclic or polycyclic heterocyclic oxy group, a carboxyl group, an optionally substituted C₁₋₆ alkylcarbonyl group, an optionally substituted C₃₋₁₀ cycloalkylcarbonyl group, an optionally substituted C₆₋₁₀ arylcarbonyl group, an optionally substituted 3- to 12-membered monocyclic or polycyclic heterocyclic carbonyl group, an optionally substituted C₁₋₆ alkoxycarbonyl group, an optionally substituted C₃₋₁₀ cycloalkoxycarbonyl group, an optionally substituted C₆₋₁₀ aryloxycarbonyl group, an optionally substituted 3- to 12-membered monocyclic or polycyclic heterocyclic oxycarbonyl group, an optionally substituted aminocarbonyl group, an optionally substituted C₁₋₆ alkylthio group, an optionally substituted C₃₋₁₀ cycloalkylthio group, an optionally substituted C₆₋₁₀ arylthio group, an optionally substituted 3- to 12-membered monocyclic or polycyclic heterocyclic thio group, a sulfinic acid group, an optionally substituted C₁₋₆ alkylsulfinyl group, an optionally substituted C₃₋₁₀ cycloalkylsulfinyl group, an optionally substituted C₆₋₁₀ arylsulfinyl group, an optionally substituted 3- to 12-membered monocyclic or polycyclic heterocyclic sulfinyl group, an optionally substituted aminosulfinyl group, a sulfonic acid group, an optionally substituted C₁₋₆ alkylsulfonyl group, an optionally substituted C₃₋₁₀ cycloalkylsulfonyl group, an optionally substituted C₆₋₁₀ arylsulfonyl group, an optionally substituted 3- to 12-membered monocyclic or polycyclic heterocyclic sulfonyl group, or an optionally substituted aminosulfonyl group, comprising the following steps (a), (b), and (c):

(a) reacting 2-hydroxy-1,4-naphthoquinone or a pharmaceutically acceptable salt thereof represented by formula (1)

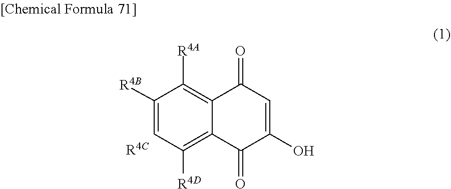

or a solvate thereof, wherein R^{4A}, R^{4B}, R^{4C}, and R^{4D} are defined as the same as above,

with a compound or a pharmaceutically acceptable salt thereof represented by formula (2a) or (2b)

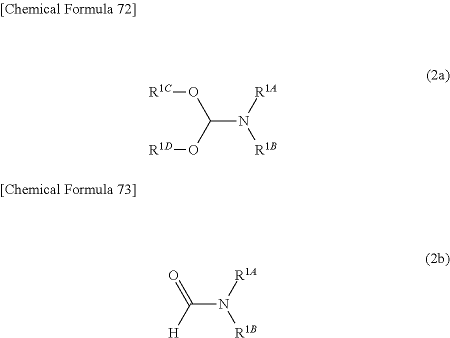

or a solvate thereof, wherein

R^{1A} and R^{1B} are defined as the same as above, and

R^{1C} and R^{1D} are the same or different, each independently a hydrogen atom, an optionally substituted C₁₋₆ alkyl group, an optionally substituted 3- to 12-membered monocyclic or polycyclic heterocyclic group, in the presence of a solvent;

(b) reacting a product of step (a) with a compound or a pharmaceutically acceptable salt thereof represented by formula (4)

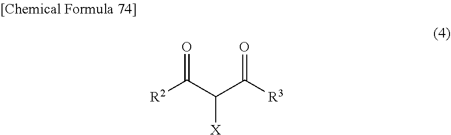

or a solvate thereof, wherein

R² is defined as the same as above,

R³ is a hydrogen atom, an optionally substituted C₁₋₁₀ alkyl group, an optionally substituted C₃₋₁₀ cycloalkyl group, an optionally substituted C₆₋₁₀ aryl group, an optionally substituted 3- to 12-membered monocyclic or polycyclic heterocyclic group, and

X is a halogen atom,

in the presence of a solvent; and
(c) heating a product obtained in step (b) in the presence of a solvent to manufacture a compound or a pharmaceutically acceptable salt thereof represented by formula (6)

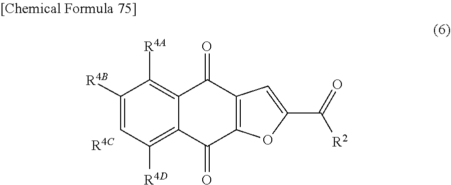

or a solvate thereof, wherein R², R^{4A}, R^{4B}, R^{4C}, and R^{4D} are defined as the same as above.
[Item 103]

A manufacturing method of a compound or a pharmaceutically acceptable salt thereof represented by formula (8)

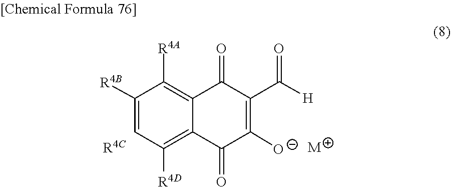

or a solvate thereof, wherein

M is Li, Na, or K, and

R^{4A}, R^{4B}, R^{4C}, and R^{4D} are the same or different, each independently a hydrogen atom, a halogen atom, a cyano group, a hydroxyl group, an optionally substituted amino group, an optionally substituted C₁₋₆ alkyl group, an optionally substituted C₃₋₁₀ cycloalkyl group, an optionally substituted C₆₋₁₀ aryl group, an optionally substituted 3- to 12-membered monocyclic or polycyclic heterocyclic group, an optionally substituted C₁₋₆ alkoxy group, an optionally substituted C₃₋₁₀ cycloalkoxy group, an optionally substituted C₆₋₁₀ aryloxy group, an optionally substituted 3- to 12-membered monocyclic or polycyclic heterocyclic oxy group, a carboxyl group, an optionally substituted C₁₋₆ alkylcarbonyl group, an optionally substituted C₃₋₁₀ cycloalkylcarbonyl group, an optionally substituted C₆₋₁₀ arylcarbonyl group, an optionally substituted 3- to 12-membered monocyclic or polycyclic heterocyclic carbonyl group, an optionally substituted C₁₋₆ alkoxycarbonyl group, an optionally substituted C₃₋₁₀ cycloalkoxycarbonyl group, an optionally substituted C₆₋₁₀ aryloxycarbonyl group, an optionally substituted 3- to 12-membered monocyclic or polycyclic heterocyclic oxycarbonyl group, an optionally substituted aminocarbonyl group, an optionally substituted C₁₋₆ alkylthio group, an optionally substituted C₃₋₁₀ cycloalkylthio group, an optionally substituted C₆₋₁₀ arylthio group, an optionally substituted 3- to 12-membered monocyclic or polycyclic heterocyclic thio group, a sulfinic acid group, an optionally substituted C₁₋₆ alkylsulfinyl group, an optionally substituted C₃₋₁₀ cycloalkylsulfinyl group, an optionally substituted C₆₋₁₀ arylsulfinyl group, an optionally substituted 3- to 12-membered monocyclic or polycyclic heterocyclic sulfinyl group, an optionally substituted aminosulfinyl group, a sulfonic acid group, an optionally substituted C₁₋₆ alkylsulfonyl group, an optionally substituted C₃₋₁₀ cycloalkylsulfonyl group, an optionally substituted C₆₋₁₀ arylsulfonyl group, an optionally substituted 3- to 12-membered monocyclic or polycyclic heterocyclic sulfonyl group, or an optionally substituted aminosulfonyl group, comprising the following steps (a) and (d):

(a) reacting 2-hydroxy-1,4-naphthoquinone or a pharmaceutically acceptable salt thereof represented by formula (1)

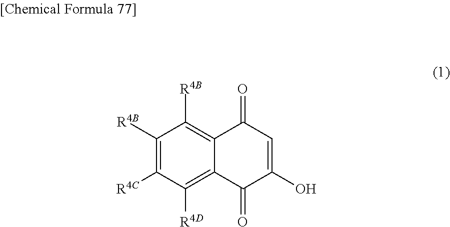

or a solvate thereof, wherein R^{4A}, R^{4B}, R^{4C}, and R^{4D} are defined as the same as above,
with a compound or a pharmaceutically acceptable salt thereof represented by formula (2a) or (2b)

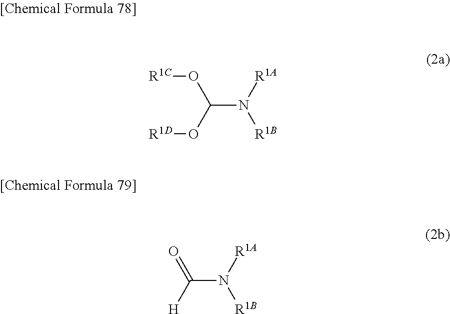

or a solvate thereof, wherein

R^{1A} and R^{1B} are defined as the same as above, and

R^{1C} and R^{1D} are the same or different, each independently a hydrogen atom, an optionally substituted C₁₋₆ alkyl group, an optionally substituted 3- to 12-membered monocyclic or polycyclic heterocyclic group, in the presence of a solvent; and

(d) reacting a product obtained in step (a) with a base in a solvent to manufacture a compound or a pharmaceutically acceptable salt thereof represented by formula (8)

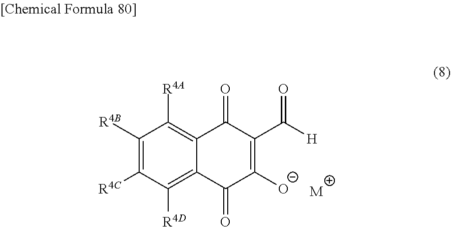

or a solvate thereof, wherein M, R^{4A}, R^{4B}, R^{4C}, and R^{4D} are defined as the same as above.

The present disclosure is intended so that one or more of the features described above can be provided not only as the explicitly disclosed combinations, but also as other combinations thereof. Additional embodiments and advantages of the present disclosure are recognized by those skilled in the art by reading and understanding the following detailed description as needed.

### Advantageous Effects of Invention

In one embodiment, the manufacturing method of the present disclosure can manufacture naphtho[2,3-b]furan-4,9-dione with a substitution at position 2 in a safer manner at a higher yield and higher purity, relative to known manufacturing methods, without using a metal or attaching or detaching a protecting group, so that a related substance in the manufacture of 2-alkylcarbonylnaphtho[2,3-b]furan-4,9-dione, which is useful as a pharmaceutical product, can be manufactured from a commercially available raw material in a safe and cost-efficient manner.

In one embodiment, the present disclosure provides a method suitable for industrial use, which has a higher yield when constructing a naphtho[2,3-b]furan-4,9-dione backbone with a substitution at position 2 and is capable of more efficiently manufacturing a product of interest more cost-efficiently from a commercially available raw material compared to known manufacturing methods.

In one embodiment, the method of the present disclosure can obtain 2-acetylnaphtho[2,3-b]furan-4,9-dione at a high yield without producing 2-acetyl-2,3-dihydronaphtho[2,3-b]furan-4,9-dione.

In one embodiment, the method of the present disclosure can manufacture a naphtho[2,3-b]furan-4,9-dione backbone with a substitution at position 2 from a cost-efficient and commercially available raw material without a need to use an expensive acetylene compound. The lack of use of a metal in a reaction would not pose the problem of residual metal in a pharmaceutical product and lead to alleviation of environment impact. Further, the method does not need to use gentotoxic substance 3-buten-2-one or aniline nor require an ultralow temperature facility or an operation to concentrate a high boiling point solvent. Furthermore, the method can provide convenience and advantages suitable for industrial use such as the need for only a filtration process without a fluid separation or concentration step in the post-processing step.

In addition, another embodiment can provide advantages such as the ability to readily perform a reaction and the like because a manufacture intermediate represented by formula (3), (5), or (7) does not need to be isolated in the reaction of the present disclosure.

### DESCRIPTION OF EMBODIMENTS

The present disclosure is described hereinafter while showing the best mode thereof. Throughout the entire specification, a singular expression should be understood as encompassing the concept thereof in the plural form, unless specifically noted otherwise. Thus, singular articles (e.g., “a”, “an”, “the”, and the like in the case of English) should also be understood as encompassing the concept thereof in the plural form, unless specifically noted otherwise. The terms used herein should also be understood as being used in the meaning that is commonly used in the art, unless specifically noted otherwise. Thus, unless defined otherwise, all terminologies and scientific technical terms that are used herein have the same meaning as the general understanding of those skilled in the art to which the disclosure pertains. In case of a contradiction, the present specification (including the definitions) takes precedence.

The presence of the following tautomers is conceivable herein for the carbonyl group and hydroxyl group of a compound represented by formula (4) and optionally pharmaceutically acceptable salt thereof. All isomers are represented by formula (4) for convenience.

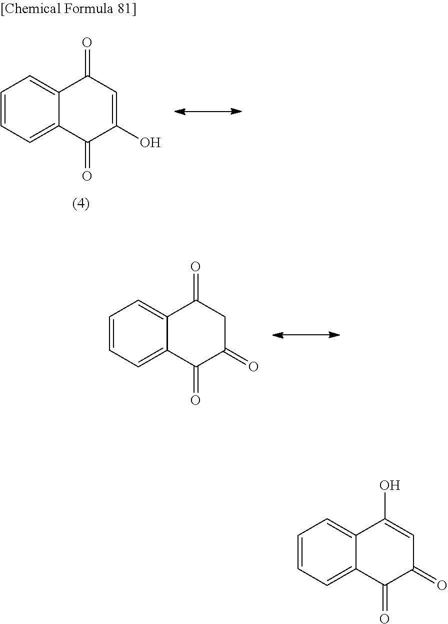 [SMILES: O=C1C=C(O)C(=O)c2ccccc21 ; O=C1CC(=O)c2ccccc2C1=O ; O=C1C=C(O)c2ccccc2C1=O]

In some cases, the presence of a tautomer is conceivable for compounds of other formulas (e.g., formula (7)) in addition to those described above for the compounds of the present disclosure. For example, it is understood that more tautomers are conceivable depending on the substituent. The present disclosure is understood to encompass any such tautomers.

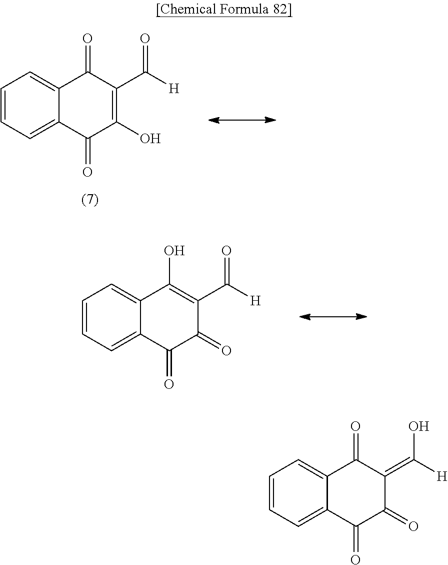 [SMILES: O=CC1=C(O)C(=O)c2ccccc2C1=O ; O=CC1=C(O)c2ccccc2C(=O)C1=O ; O=C1C(=O)c2ccccc2C(=O)/C1=C\O]

The compound of the present disclosure can also be present in the form of a solvate (e.g., hydrate). Thus, the compounds of the present disclosure also encompass solvates (e.g., hydrates) of the compounds represented by formula (1), (2a), (2b), (3), (4), (5), (6), (7), and (8) and tautomers thereof, and optionally pharmaceutically acceptable salts thereof.

The compounds represented by formulas (1), (2a), (2b), (3), (4), (5), (6), (7), and (8) may have one or optionally more asymmetric carbon atoms in some cases, resulting in geometrical isomerism or axial chirality, so that the compounds can be present as several types of stereoisomers. In the present disclosure, the compounds of the present disclosure also encompass such stereoisomers, and mixtures and racemates thereof.

Deuterated compounds prepared by converting any one or more of ¹H (hydrogen atom) of a compound represented by formula (1), (2a), (2b), (3), (4), (5), (6), (7), or (8) to ²H (D: deuterated atom) are also encompassed by the compounds represented by formula (1), (2a), (2b), (3), (4), (5), (6), (7), or (8).

The compounds represented by formulas (1), (2a), (2b), (3), (4), (5), (6), (7), and (8) and tautomers thereof, and optionally pharmaceutically acceptable salts thereof obtained as a crystal can have a crystalline polymorphism. The compounds of the present disclosure encompass any crystalline form.

The number of carbons in the definition of a “substituent” can be denoted herein as, for example, “C₁-6” or the like. Specifically, the expression “C₁₋₆ alkyl” is synonymous with an alkyl group having 1 to 6 carbons. As used herein, a substituent that is not expressly described with the term “optionally substituted” or “substituted” refers to an “unsubstituted” substituent. For example, “C₁₋₆ alkyl” means that the substituent is “unsubstituted”.

As used herein, the term “group” refers to a monovalent group. For example, “alkyl group” refers to a monovalent saturated hydrocarbon group. The term “group” may also be omitted in the descriptions of substituents herein.

The terms that are used herein are explained hereinafter.

The number of substituents in a group defined as “optionally substituted” or “substituted” is not particularly limited herein, as long as a substitution is possible. The number of substituents is 0, 1, or multiple substituents. Moreover, unless noted otherwise, the description for each group is also applicable when the group is a part of or a substituent of another group.

The substituent in “optionally substituted . . . ” can be appropriately selected herein depending on the substituted group. For example, “optionally substituted C₁₋₁₀ alkyl group” refers to the C₁₋₁₀ alkyl group which is optionally substituted at any substitutable position with a fluorine atom, chlorine atom, methoxy group, or hydroxyl group. Examples thereof include a methyl group, an ethyl group, an n-propyl group, an isopropyl group, a difluoromethyl group, a trifluoromethyl group, a 2-fluoroethyl group, a 2-methoxyethyl group, and the like. It is preferably a methyl group, an ethyl group, a difluoromethyl group, a trifluoromethyl group, a 2-fluoroethyl group, or a 2-methyoxyethyl group.

Examples of substituents in “optionally substituted C₁₋₆ alkoxy group”, “optionally substituted C₆₋₁₀ aryl group”, “optionally substituted C₁₋₁₀ alkylcarbonyl group”, “optionally substituted C₁₋₁₀ alkyloxycarbonyl group”, “optionally substituted C₆₋₁₀ arylcarbonyl group”, “optionally substituted C₃₋₁₀ cycloalkyl group”, “optionally substituted cyclic ketal”, “optionally substituted 1,3-dioxolane”, “optionally substituted 3- to 12-membered monocyclic or polycyclic heterocyclic group”, “optionally substituted C₂₋₁₀ alkenyl group”, “optionally substituted C₂₋₁₀ alkenyloxycarbonyl group”, “optionally substituted amino group”, “optionally substituted aminocarbonyl group”, “optionally substituted C₁₋₁₀ alkylsulfonyl group”, “optionally substituted C₆₋₁₀ arylsulfonyl group”, “optionally substituted aminocarbonyl group”, “optionally substituted phenyl group”, “optionally substituted iodonio group”, “optionally substituted sulfonyloxy group”, or “optionally substituted phosphoryloxy group” include substituents selected from the following substituent group (a). Each of these substituents can replace one or more substituents at any substitutable position.

Substituents (a): halogen atom, cyano group, nitro group, amino group, methylamino group, dimethylamino group, methanesulfonylamino group, acetyl group, propionyl group, methoxycarbonyl group, benzoyl group, C₁₋₃ alkyl group, C₁₋₃ alkoxy group, and 3- to 7-membered heterocyclic group. Substituents (a) can also be selected from a halogen atom, cyano group, nitro group, amino group, methylamino group, dimethylamino group, methanesulfonylamino group, acetyl group, propionyl group, methoxycarbonyl group, benzoyl group, C₁₋₆ alkyl group, C₁₋₆ alkoxy group, and 3- to 7-membered heterocyclic group.

Examples of a “halogen atom” herein include a fluorine atom, a chlorine atom, a bromine atom, and an iodine atom. A halogen atom is preferably a chlorine atom or a bromine atom, and more preferably a chlorine atom.

“Alkyl group” refers to a linear or branched, saturated hydrocarbon group. For example, “C₁_₄ alkyl group” or “C₆ alkyl group” refers to an alkyl group having 1 to 4 or 6 carbon atoms. The same applies to other numbers. “C₁₋₁₀ alkyl group” is preferably a “C₁₋₆ alkyl group” and more preferably a “C₁_₄ alkyl group”. Specific examples of “C₁₋₁₀ alkyl group” include a methyl group, an ethyl group, a propyl group, a 1-methylethyl group, a butyl group, a 2-methylpropyl group, a 1-methylpropyl group, a 1,1-dimethylethyl group, a pentyl group, a 3-methylbutyl group, a 2-methylbutyl group, a 2,2-dimethylpropyl group, a 1-ethylpropyl group, a 1,1-dimethylpropyl group, a hexyl group, a 4-methylpentyl group, a 3-methylpentyl group, a 2-methylpentyl group, a 1-methylpentyl group, a hexyl group, a heptyl group, an octyl group, a nonyl group, a decyl group, and the like. Specific examples of “C₁₋₆ alkyl group” include instances with 1 to 6 carbon atoms in the specific examples of “C₁₋₁₀ alkyl group”. Specific examples of “C₁₋₄ alkyl group” include instances with 1 to 4 carbon atoms in the specific examples of “C₁₋₁₀ alkyl group”.

“C₃₋₁₀ cycloalkyl group” refers to a cyclic alkyl having 3 to 10 carbon atoms, including cyclic alkyl with a partially bridged structure. “C₃₋₁₀ cycloalkyl group” is preferably a “C₃₋₇ cycloalkyl group” and more preferably a “C₄₋₆ cycloalkyl group”. Specific examples of “C₃₋₁₀ cycloalkyl group” include a cyclopropyl group, a cyclobutyl group, a cyclopentyl group, a cyclohexyl group, a cycloheptyl group, a cyclooctyl group, an adamantyl group, and the like. Specific examples of “C₃₋₇ cycloalkyl group” include a cyclopropyl group, a cyclobutyl group, a cyclopentyl group, a cyclohexyl group, a cycloheptyl group, and the like.

“C₁₋₆ alkoxy group” refers to a “C₁₋₆ alkyloxy group”, and the “C₁₋₆ alkyl” moiety is defined the same as the “C₁₋₆ alkyl group” described above. “C₁₋₆ alkoxy group” is preferably a “C₁_₄ alkoxy group”. Specific examples of “C₁₋₆ alkoxy group” include a methoxy group, an ethoxy group, a propoxy group, a 1-methylethoxy group, a butoxy group, a 2-methylpropoxy group, a 1-methylpropoxy group, a 1,1-dimethylethoxy group, a pentyloxy group, a 3-methylbutoxy group, a 2-methylbutoxy group, a 2,2-dimethylpropoxy group, a 1-ethylpropoxy group, a 1,1-dimethylpropoxy group, a hexyloxy group, a 4-methylpentyloxy group, a 3-methylpentyloxy group, a 2-methylpentyloxy group, a 1-methylpentyloxy group, a 3,3-dimethylbutoxy group, a 2,2-dimethylbutoxy group, a 1,1-dimethylbutoxy group, a 1,2-dimethylbutoxy group, and the like.

“C₃₋₁₀ cycloalkoxy group” refers to “C₃₋₁₀ cycloalkyloxy group”, and the “C₃₋₁₀ cycloalkyl” moiety is defined the same as the “C₃₋₁₀ cycloalkyl group” described above. “C₃₋₁₀ cycloalkoxy group” is preferably a “C₃₋₇ cycloalkoxy group”, and more preferably a “C₄₋₆ cycloalkoxy group”.

The “C₃₋₁₀ cycloalkoxy” moiety of “C₃₋₁₀ cycloalkoxycarbonyl group” is defined the same as the “C₃₋₁₀ cycloalkoxy group” described above.

“C₆₋₁₀ aryl group” refers to an aromatic hydrocarbon with 6 to 10 carbon atoms. Specific examples of “C₆₋₁₀ aryl group” include a phenyl group, a 1-naphthyl group, a 2-naphthyl group, and the like. A phenyl group is particularly preferred.

The “C₆₋₁₀ aryl” moiety of “C₆₋₁₀ aryloxy group” is defined the same as the “C₆₋₁₀ aryl group” described above. A C₆₋₁₀ aryloxy group is preferably a phenoxy group.

The “C₁₋₆ alkyl” moiety of “C₁₋₆ alkylcarbonyl group” is defined the same as the “C₁₋₆ alkyl group” described above. “C₁₋₆ alkylcarbonyl group” is preferably a “C₁_4 alkylcarbonyl group”. Specific examples of “C₁₋₆ alkylcarbonyl group” include a methylcarbonyl group (acetyl group), an ethylcarbonyl group, a propylcarbonyl group, a 1-methylethylcarbonyl group, a butylcarbonyl group, a 2-methylpropylcarbonyl group, a 1-methylpropylcarbonyl group, a 1,1-dimethylethylcarbonyl group, and the like.

The “C₃₋₁₀ cycloalkyl” moiety of “C₃₋₁₀ cycloalkylcarbonyl group” is defined the same as the “C₃₋₁₀ cycloalkyl group” described above.

The “C₆₋₁₀ aryl” moiety of “C₆₋₁₀ arylcarbonyl group” is defined the same as the “C₆₋₁₀ aryl group” described above. Specific examples of “C₆₋₁₀ arylcarbonyl group” include a phenylcarbonyl group, a 1-naphthylcarbonyl group, a 2-naphthylcarbonyl group, and the like. It is preferably a phenyl carbonyl group.

The “C₁₋₆ alkoxy” moiety of “C₁₋₆ alkoxycarbonyl group” is defined the same as the “C₁₋₆ alkoxy group” described above. “C₁₋₆ alkoxycarbonyl group” is preferably a “C₁₋₄ alkoxycarbonyl group”. Specific examples of “C₁₋₆ alkyloxycarbonyl group” include a methoxycarbonyl group, an ethoxycarbonyl group, a propoxycarbonyl group, a 1-methylethoxycarbonyl group, a butoxycarbonyl group, a 2-methylpropoxycarbonyl group, a 1-methylpropoxycarbonyl group, a 1,1-dimethylethoxycarbonyl group, a pentyloxycarbonyl group, a 3-methylbutoxycarbonyl group, a 2-methylbutoxycarbonyl group, a 2,2-dimethylpropoxycarbonyl group, a 1-ethylpropoxycarbonyl group, a 1,1-dimethylpropoxycarbonyl group, a hexyloxycarbonyl group, a 4-methylpentyloxycarbonyl group, a 3-methylpentyloxycarbonyl group, a 2-methylpentyloxycarbonyl group, a 1-methylpentyloxycarbonyl group, a 3,3-dimethylbutoxycarbonyl group, a 2,2-dimethylbutoxycarbonyl group, a 1,1-dimethylbutoxycarbonyl group, a 1,2-dimethylbutoxycarbonyl group, and the like.

The “C₆₋₁₀ aryloxy” moiety of “C₆₋₁₀ aryloxycarbonyl group” is defined the same as the “C₆₋₁₀ aryloxy group” described above. “C₆₋₁₀ aryloxycarbonyl group” is preferably phenoxycarbonyl.

Examples of “3- to 12-membered monocyclic or polycyclic heterocyclic group” include monocyclic or polycyclic heterocyclic groups comprising 1 to 4 atoms independently selected from the group consisting of a nitrogen atom, an oxygen atom, and a sulfur atom. It is preferably a 3- to 10-membered group, more preferably a 3- to 8-membered group, and still more preferably a 5- or 6-membered group. Each of the nitrogen atom, oxygen atom, and sulfur atom is an atom that constitutes a ring. The heterocyclic group may be either saturated or partially unsaturated, and is preferably a saturated heterocyclic group. Specific examples of “heterocyclic group” include an oxiranyl group, an aziridinyl group, an azetidinyl group, a pyranyl group, a tetrahydrofuranyl group, a pyrrolidinyl group, an imidazolidinyl group, a piperidinyl group, a piperadinyl group, a morpholinyl group, a thiomorpholinyl group, a dioxothiomorpholinyl group, a hexamethyleniminyl group, an oxazolidinyl group, a thiazolidinyl group, an imidazolidinyl group, an oxoimidazolidinyl group, a dioxoimidazolidinyl group, an oxooxazolidinyl group, a dioxooxazolidinyl group, a dioxothiazolidinyl group, a tetrahydropyridyl group, an oxetanyl group, a dioxanyl group, a tetrahydrothiopyranyl group, a tetrahydropyranyl group, and the like. It should be noted that the group also encompasses a heterocyclic group having a bridged structure. For such a group, a nitrogen atom constituting the ring cannot be at a position to be attached in “the group”. In other words, the group does not encompass the concepts of, for example, a 1-pyrrolidino group and the like.

The “3- to 12-membered monocyclic or polycyclic heterocycle” moiety of “3- to 12-membered monocyclic or polycyclic heterocyclic oxy group” is defined the same as the “3- to 12-membered monocyclic or polycyclic heterocyclic group” described above.

The “3- to 12-membered monocyclic or polycyclic heterocycle” moiety of “3- to 12-membered monocyclic or polycyclic heterocyclic carbonyl group” is defined the same as the “3- to 12-membered monocyclic or polycyclic heterocyclic group” described above.

The “3- to 12-membered monocyclic or polycyclic heterocycle” moiety of “3- to 12-membered monocyclic or polycyclic heterocyclic oxycarbonyl group” is defined the same as the “3- to 12-membered monocyclic or polycyclic heterocyclic group” described above.

“Aminocarbonyl group” refers to a group in which an “amino group” is bound to a carbonyl group. The “amino” therein refers to nitrogen atom unsubstituted amino, mono-substituted amino, di-substituted amino, or 3- to 12-membered cyclic amino. Specific examples thereof include a methylaminocarbonyl group, a cyclopropylaminocarbonyl group, a dimethylaminocarbonyl group, a dicyclopropylaminocarbonyl group, a phenylaminocarbonyl group, and the like. It is preferably a phenylaminocarbonyl group.

The “C₁₋₁₀ alkyl” moiety of a “C₁₋₁₀ alkylsulfonyl group” is defined the same as the “C₁₋₁₀ alkyl group” described above. “C₁₋₁₀ alkylsulfonyl group” is preferably a “C₁₋₆ alkylsulfonyl group”. Specific examples of “C₁₋₆ alkylsulfonyl group” include a methanesulfonyl group, an ethanesulfonyl group, a propanesulfonyl group, a 1-methylethanesulfonyl group, a butanesulfonyl group, a 2-methylpropanesulfonyl group, a 1-methylpropanesulfonyl group, a 1,1-dimethylethanesulfonyl group, and the like.

The “C₆₋₁₀ aryl” moiety of a “C₆₋₁₀ arylsulfonyl group” is defined the same as the “C₆₋₁₀ aryl group” described above. “C₆₋₁₀ arylsulfonyl group” is preferably a “C₆ arylsulfonyl group”. Specific examples of “C₆ arylsulfonyl group” include a benzenesulfonyl group (the benzenesulfonyl group is defined the same as a phenylsulfonyl group), a p-toluenesulfonyl group, and the like.

The “C₁₋₆ alkyl” moiety of “C₁₋₆ alkylthio group” is defined the same as the “C₁₋₆ alkyl group” described above.

The “C₃₋₁₀ cycloalkyl” moiety of “C₃₋₁₀ cycloalkylthio group” is defined the same as the “C₃₋₁₀ cycloalkyl group” described above.

The “C₆₋₁₀ aryl” moiety of “C₆₋₁₀ arylthio group” is defined the same as the “C₆₋₁₀ aryl group” described above.

The “3- to 12-membered monocyclic or polycyclic heterocycle” moiety of “3- to 12-membered monocyclic or polycyclic heterocyclic thio group” is defined the same as the “3- to 12-membered monocyclic or polycyclic heterocyclic group” described above.

The “C₁₋₆ alkyl” moiety of “C₁₋₆ alkylsulfinyl group” is defined the same as the “C₁_₆ alkylsulfinyl group” described above.

The “C₃₋₁₀ cycloalkyl” moiety of “C₃₋₁₀ cycloalkylsulfinyl group” is defined the same as the “C₃₋₁₀ cycloalkyl group” described above.

The “C₆₋₁₀ aryl” moiety of “C₆₋₁₀ arylsulfinyl group” is defined the same as the “C₆₋₁₀ aryl group” described above.

The “3- to 12-membered monocyclic or polycyclic heterocycle” moiety of “3- to 12-membered monocyclic or polycyclic heterocyclic sulfinyl group” is defined the same as the “3- to 12-membered monocyclic or polycyclic heterocyclic group” described above.

The “amino” moiety of “aminosulfinyl group” is defined the same as the “amino group” described above.

The “C₃₋₁₀ cycloalkyl” moiety of “C₃₋₁₀ cycloalkylsulfinyl group” is defined the same as the “C₃₋₁₀ cycloalkyl group” described above.

The “3- to 12-membered monocyclic or polycyclic heterocycle” moiety of “3- to 12-membered monocyclic or polycyclic heterocyclic sulfonyl group” is defined the same as the “3- to 12-membered monocyclic or polycyclic heterocyclic group”.

The “amino” moiety of “aminosulfonyl group” is defined the same as the “amino group” described above.

A phase transfer catalyst such as tetrabutylammonium salt may be added as needed to the reaction of the present disclosure to the extent that the reaction is not adversely affected.

“Base” encompasses both organic bases and inorganic bases.

Specific examples of “organic base” include triethylamine, N,N,N′,N′-tetramethylethane-1,2-diamine, N,N-dimethylaniline, N,N-diisopropylethylamine, N-methylpyrrolidine, N-methylpiperidine, 1,4-diazabicyclo[2.2.2]octane, N-methylmorpholine, diazabicycloundecene, methylamine, diisopropylamine, pyrimidine, and pyridine. An organic base is more preferably triethylamine, diisopropylethylamine N,N,N′,N′-tetramethylethane-1,2-diamine, 1,4-diazabicyclo[2.2.2]octane, N-methylpiperidine, pyrimidine, and pyridine, still more preferably N,N,N′,N′-tetramethylethane-1,2-diamine, 1,4-diazabicyclo[2.2.2]octane, N-methylpiperidine, pyrimidine, and pyridine, and most preferably N,N-diisopropylethylamine.

Specific examples of “inorganic base” include, but are not limited to, ammonium, lithium hydroxide, sodium hydroxide, calcium hydroxide, barium hydroxide, sodium carbonate, sodium hydrogen carbonate, potassium carbonate, cesium carbonate, mixtures thereof, and the like. An inorganic base is preferably lithium hydroxide, sodium hydroxide, sodium carbonate, potassium carbonate, or cesium carbonate, more preferably sodium carbonate, potassium carbonate, sodium hydrogen carbonate, or cesium carbonate, and most preferably potassium carbonate.

“Alcohol-based solvent” refers to a solvent, which is a compound comprising one or more hydroxyl groups in a molecule and is a liquid at a reaction temperature, having a property of dissolving or dispersing a reactant. Specific examples of “alcohol-based solvent” include methanol, ethanol, 1-propanol, 2-propanol, 1-butanol, 2-butanol, 3-methyl-1-butanol, 2-methyl-1-propanol, and tert-butyl alcohol. An alcohol-based solvent is preferably methanol.

“Amide-based solvent” refers to a solvent, which is a compound comprising one or more amide bonds in a molecule and is a liquid at a reaction temperature, having a property of dissolving or dispersing a reactant. Specific examples of “amide-based solvent” include N,N-dimethylacetamide, N,N-dimethylformamide, N,N-diethylformamide, N-methyl-2-pyrrolidone, tetramethylurea, hexamethylphosphoric triamide, and the like. An “amide-based solvent” is preferably N,N-dimethylformamide or N-methyl-2-pyrrolidone, and more preferably N-methyl-2-pyrrolidine.

As used herein, it is understood that the expression of “at least one independently selected from . . . ” for an expression for a solvent or the like encompasses mixtures of two or more options when two or more are selected from the options.

As used herein, the term “pharmaceutically acceptable salt” refers to a salt prepared from a pharmaceutically acceptable acid (including inorganic and organic acids) unless specifically noted otherwise. In addition, “optionally pharmaceutically acceptable salt (thereof)” means that this can be a salt which is optionally pharmaceutically acceptable. For example, this means that a salt, which is not pharmaceutically acceptable, up to a certain stage, can be used for the manufacture of an intermediate. Examples of pharmaceutically acceptable salts include, but are not limited to, acetic acid, alginic acid, anthranilic acid, benzenesulfonic acid, benzoic acid, camphorsulfonic acid, citric acid, ethenesulfonic acid, formic acid, fumaric acid, furoic acid, gluconic acid, glutamic acid, glucorenic acid, galacturonic acid, glycidic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phenylacetic acid, propionic acid, phosphoric acid, salicylic acid, stearic acid, succinic acid, sulfanilic acid, sulfuric acid, tartaric acid, p-toluenesulfonic acid, and the like.

“Purification” refers to any act that enhances the purity of a substance of interest and reduces the concentration of substances other than the substance of interest below the concentration prior to the purification. Various methods such as precipitation, recrystallization, sublimation, distillation, solvent extraction, use of molecular sieve, and application of various chromatographies can be used for purification. Purification does not include filtration using a filter paper or Celite.

Preferred R^{1A}, R^{1B}, R^{1C}, R^{1D}, R², R³, R^{4A}, R^{4B}, R^{4C}, and R^{4D} in the compounds of the present disclosure represented by formulas (1), (2a), (2b), (3), (4), (5), (6), (7), and (8) are the following, but the technical scope of the present disclosure is not limited to the scope of compounds listed below.

R^{1A} and R^{1B} are the same or different, each independently

(1) a hydrogen atom,
(2) an optionally substituted C₁₋₁₀ alkyl group, or
(3) an optionally substituted C₃₋₁₀ cycloalkyl group, wherein R^{1A} and R^{1B} are not simultaneously a hydrogen atom.

R^{1A} and R^{1B} are preferably the same or different, each independently

(1) a hydrogen atom,
(2) a C₁₋₆ alkyl group, wherein the alkyl group is optionally substituted with 1 to 3 groups independently selected from the group consisting of a phenyl group, a halogen atom, a hydroxyl group, and a C₁₋₆ alkoxy group, or (3) a C₃₋₁₀ cycloalkyl group, wherein the cycloalkyl group is optionally substituted with 1 to 3 groups independently selected from the group consisting of a C₁₋₆ alkyl group, a phenyl group, a halogen atom, a hydroxyl group, and a C₁₋₆ alkoxy group,

wherein R^{1A} and R^{1B} are not simultaneously a hydrogen atom.

R^{1A} and R^{1B} are more preferably the same or different, each independently

(1) a hydrogen atom,
(2) a C₁₋₆ alkyl group, or
(3) a C₃₋₁₀ cycloalkyl group,

wherein R^{1A} and R^{1B} are not simultaneously a hydrogen atom.

R^{1A} and R^{1B} are still more preferably the same or different, each independently a hydrogen atom or a C₁₋₃ alkyl group, wherein R^{1A} and R^{1B} are not simultaneously a hydrogen atom.

R^{1A} and R^{1B} are still more preferably each independently a methyl group, an ethyl group, a propyl groups, or an isopropyl group.

R^{1A} and R^{1B} are most preferably a methyl group.

R² is

(1) a hydrogen atom,
(2) an optionally substituted C₁₋₁₀ alkyl group,
(3) an optionally substituted C₃₋₁₀ cycloalkyl group,
(4) an optionally substituted C₆₋₁₀ aryl group, or
(5) an optionally substituted 3- to 12-membered monocyclic or polycyclic heterocyclic group.

R² is preferably an optionally substituted C₁₋₁₀ alkyl group.

R² is more preferably a C₁₋₁₀ alkyl group optionally substituted with 1 to 3 groups independently selected from the group consisting of a phenyl group, a halogen atom, a hydroxyl group, and a C₁₋₆ alkoxy group.

R² is still more preferably a C₁₋₆ alkyl group optionally substituted with 1 to 3 groups independently selected from the group consisting of a hydroxyl group and a C₁₋₆ alkoxy group.

R² is still more preferably a C₁_₃ alkyl group.

R² is most preferably a methyl group.

R³ is

(1) a hydrogen atom,
(2) an optionally substituted C₁₋₁₀ alkyl group,
(3) an optionally substituted C₃₋₁₀ cycloalkyl group,
(4) an optionally substituted C₆₋₁₀ aryl group, or
(5) an optionally substituted 3- to 12-membered monocyclic or polycyclic heterocyclic group.

R³ is preferably

(1) a hydrogen atom, or
(2) a C₁₋₁₀ alkyl group, a C₃₋₁₀ cycloalkyl group, a C₆₋₁₀ aryl group, a 3- to 12-membered monocyclic or polycyclic heterocyclic group (wherein the alkyl group is optionally substituted with 1 to 3 groups independently selected from the group consisting of a phenyl group, a halogen atom, a hydroxyl group, and a C₁₋₆ alkoxy group, and the cycloalkyl group, aryl group, or heterocyclic group is optionally substituted with 1 to 3 groups independently selected from the group consisting of a C₁₋₁₀ alkyl group, a phenyl group, a halogen atom, a hydroxyl group, and a C₁₋₆ alkoxy group.

R³ is more preferably a C₁₋₆ alkyl group optionally substituted with 1 to 3 groups independently selected from the group consisting of a halogen atom, a hydroxyl group, and a C₁₋₆ alkoxy group.

R³ is still more preferably a C₁₋₆ alkyl group.

R³ is most preferably a methyl group.

R^{4A}, R^{4B}, R^{4C}, and R^{4D} are the same or different, each independently

(1) a hydrogen atom,
(2) a halogen atom,
(3) a cyano group,
(4) a hydroxyl group,
(5) an optionally substituted amino group,
(6) an optionally substituted C₁₋₆ alkyl group,
(7) an optionally substituted C₃₋₁₀ cycloalkyl group,
(8) an optionally substituted C₆₋₁₀ aryl group,
(9) an optionally substituted 3- to 12-membered monocyclic or polycyclic heterocyclic group,
(10) an optionally substituted C₁₋₆ alkoxy group,
(11) an optionally substituted C₃₋₁₀ cycloalkoxy group,
(12) an optionally substituted C₆₋₁₀ aryloxy group,
(13) an optionally substituted 3- to 12-membered monocyclic or polycyclic heterocyclic oxy group,
(14) a carboxyl group,
(15) an optionally substituted C₁₋₆ alkylcarbonyl group,
(16) an optionally substituted C₃₋₁₀ cycloalkylcarbonyl group,
(17) an optionally substituted C₆₋₁₀ arylcarbonyl group,
(18) an optionally substituted 3- to 12-membered monocyclic or polycyclic heterocyclic carbonyl group,
(19) an optionally substituted C₁₋₆ alkoxycarbonyl group,
(20) an optionally substituted C₃₋₁₀ cycloalkoxycarbonyl group,
(21) an optionally substituted C₆₋₁₀ aryloxycarbonyl group,
(22) an optionally substituted 3- to 12-membered monocyclic or polycyclic heterocyclic oxycarbonyl group,
(23) an optionally substituted aminocarbonyl group,
(24) an optionally substituted C₁₋₆ alkylthio group,
(25) an optionally substituted C₃₋₁₀ cycloalkylthio group,
(26) an optionally substituted C₆₋₁₀ arylthio group,
(27) an optionally substituted 3- to 12-membered monocyclic or polycyclic heterocyclic thio group,
(28) a sulfinic acid group,
(29) an optionally substituted C₁₋₆ alkylsulfinyl group,
(30) an optionally substituted C₃₋₁₀ cycloalkylsulfinyl group,
(31) an optionally substituted C₆₋₁₀ arylsulfinyl group,
(32) an optionally substituted 3- to 12-membered monocyclic or polycyclic heterocyclic sulfinyl group,
(33) an optionally substituted aminosulfinyl group,
(34) a sulfonic acid group,
(35) an optionally substituted C₁₋₆ alkylsulfonyl group,
(36) an optionally substituted C₃₋₁₀ cycloalkylsulfonyl group,
(37) an optionally substituted C₆₋₁₀ arylsulfonyl group,
(38) an optionally substituted 3- to 12-membered monocyclic or polycyclic heterocyclic sulfonyl group, or
(39) an optionally substituted aminosulfonyl group.

R^{4A}, R^{4B}, R^{4C}, and R^{4D} are preferably the same or different, each independently

(1) a hydrogen atom,
(2) a halogen atom,
(3) a cyano group,
(4) a hydroxyl group,
(5) an amino group optionally substituted with a C₁₋₆ alkyl group,
(6) a C₁₋₆ alkyl group, wherein the alkyl group is optionally substituted with 1 to 3 groups independently selected from the group consisting of a phenyl group, a halogen atom, a hydroxyl group, and a C₁₋₆ alkoxy group,
(7) a C₃₋₁₀ cycloalkyl group, wherein the cycloalkyl group is optionally substituted with 1 to 3 groups independently selected from the group consisting of a C₁₋₆ alkyl group, a phenyl group, a halogen atom, a hydroxyl group, and a C₁₋₆ alkoxy group,
(8) a C₆₋₁₀ aryl group, wherein the aryl group is optionally substituted with 1 to 3 groups independently selected from the group consisting of a C₁₋₆ alkyl group, a phenyl group, a halogen atom, a hydroxyl group, and a C₁₋₆ alkoxy group,
(9) a 3- to 12-membered monocyclic or polycyclic heterocyclic group, wherein the heterocyclic group is optionally substituted with 1 to 3 groups independently selected from the group consisting of a C₁₋₆ alkyl group, a phenyl group, a halogen atom, a hydroxyl group, and a C₁₋₆ alkoxy group,
(10) a C₁₋₆ alkoxy group, wherein the alkoxy group is optionally substituted with 1 to 3 groups independently selected from the group consisting of a phenyl group, a halogen atom, a hydroxyl group, and a C₁₋₆ alkoxy group,
(11) a C₃₋₁₀ cycloalkoxy group, wherein the cycloalkoxy group is optionally substituted with 1 to 3 groups independently selected from the group consisting of a C₁₋₆ alkyl group, a phenyl group, a halogen atom, a hydroxyl group, and a C₁₋₆ alkoxy group,
(12) a C₆₋₁₀ aryloxy group, wherein the aryl group is optionally substituted with 1 to 3 groups independently selected from the group consisting of a C₁₋₆ alkyl group, a phenyl group, a halogen atom, a hydroxyl group, and a C₁₋₆ alkoxy group,
(13) a 3- to 12-membered monocyclic or polycyclic heterocyclic oxy group, wherein the heterocycle is optionally substituted with 1 to 3 groups independently selected from the group consisting of a C₁₋₆ alkyl group, a phenyl group, a halogen atom, a hydroxyl group, and a C₁₋₆ alkoxy group,
(14) a carboxyl group,
(15) a C₁₋₆ alkylcarbonyl group, wherein the alkyl is optionally substituted with 1 to 3 groups independently selected from the group consisting of a phenyl group, a halogen atom, a hydroxyl group, and a C₁₋₆ alkoxy group,
(16) a C₃₋₁₀ cycloalkylcarbonyl group, wherein the cycloalkyl is optionally substituted with 1 to 3 groups independently selected from the group consisting of a C₁₋₆ alkyl group, a phenyl group, a halogen atom, a hydroxyl group, and a C₁₋₆ alkoxy group,
(17) a C₆₋₁₀ arylcarbonyl group, wherein the aryl is optionally substituted with 1 to 3 groups independently selected from the group consisting of a C₁₋₆ alkyl group, a phenyl group, a halogen atom, a hydroxyl group, and a C₁₋₆ alkoxy group,
(18) a 3- to 12-membered monocyclic or polycyclic heterocyclic carbonyl group, wherein the heterocycle is optionally substituted with 1 to 3 groups independently selected from the group consisting of a C₁₋₆ alkyl group, a phenyl group, a halogen atom, a hydroxyl group, and a C₁₋₆ alkoxy group,
(19) a C₁₋₆ alkoxycarbonyl group, wherein the alkoxy is optionally substituted with 1 to 3 groups independently selected from the group consisting of a phenyl group, a halogen atom, a hydroxyl group, and a C₁₋₆ alkoxy group,
(20) a C₃₋₁₀ cycloalkoxycarbonyl group, wherein the cycloalkoxy is optionally substituted with 1 to 3 groups independently selected from the group consisting of a C₁₋₆ alkyl group, a phenyl group, a halogen atom, a hydroxyl group, and a C₁₋₆ alkoxy group,
(21) a C₆₋₁₀ aryloxycarbonyl group, wherein the aryl is optionally substituted with 1 to 3 groups independently selected from the group consisting of a C₁₋₆ alkyl group, a phenyl group, a halogen atom, a hydroxyl group, and a C₁₋₆ alkoxy group,
(22) a 3- to 12-membered monocyclic or polycyclic heterocyclic oxycarbonyl group, wherein the heterocycle is optionally substituted with 1 to 3 groups independently selected from the group consisting of a C₁₋₆ alkyl group, a phenyl group, a halogen atom, a hydroxyl group, and a C₁₋₆ alkoxy group, or
(23) an aminocarbonyl group optionally substituted with a C₁₋₆ alkyl group.

R^{4A}, R^{4B}, R^{4C}, and R^{4D} are more preferably the same or different, each independently

(1) a hydrogen atom,
(2) a halogen atom,
(3) a cyano group,
(4) a hydroxyl group,
(5) an amino group optionally substituted with a C₁₋₆ alkyl group,
(6) a C₁₋₆ alkyl group, wherein the alkyl group is optionally substituted with 1 to 3 groups independently selected from the group consisting of a halogen atom, a hydroxyl group, and a C₁₋₆ alkoxy group,
(7) a C₃₋₁₀ cycloalkyl group, wherein the cycloalkyl group is optionally substituted with 1 to 3 groups independently selected from the group consisting of a C₁₋₆ alkyl group, a a halogen atom, a hydroxyl group, and a C₁₋₆ alkoxy group,
(8) a C₆₋₁₀ aryl group, wherein the aryl group is optionally substituted with 1 to 3 groups independently selected from the group consisting of a C₁₋₆ alkyl group, a halogen atom, a hydroxyl group, and a C₁₋₆ alkoxy group,
(9) a 3- to 12-membered monocyclic or polycyclic heterocyclic group, wherein the heterocyclic group is optionally substituted with 1 to 3 groups independently selected from the group consisting of a C₁₋₆ alkyl group, a halogen atom, a hydroxyl group, and a C₁₋₆ alkoxy group,
(10) a C₁₋₆ alkoxy group, wherein the alkoxy group is optionally substituted with 1 to 3 groups independently selected from the group consisting of a halogen atom, a hydroxyl group, and a C₁₋₆ alkoxy group,
(11) a C₃₋₁₀ cycloalkoxy group, wherein the cycloalkoxy group is optionally substituted with 1 to 3 groups independently selected from the group consisting of a C₁₋₆ alkyl group, a halogen atom, a hydroxyl group, and a C₁₋₆ alkoxy group,
(12) a C₆₋₁₀ aryloxy group, wherein the aryl group is optionally substituted with 1 to 3 groups independently selected from the group consisting of a C₁₋₆ alkyl group, a halogen atom, a hydroxyl group, and a C₁₋₆ alkoxy group,
(13) a 3- to 12-membered monocyclic or polycyclic heterocyclic oxy group, wherein the heterocycle is optionally substituted with 1 to 3 groups independently selected from the group consisting of a C₁₋₆ alkyl group, a a halogen atom, a hydroxyl group, and a C₁₋₆ alkoxy group,
(14) a carboxyl group,
(15) a C₁₋₆ alkylcarbonyl group, wherein the alkyl is optionally substituted with 1 to 3 groups independently selected from the group consisting of a halogen atom, a hydroxyl group, and a C₁₋₆ alkoxy group, or
(16) a C₃₋₁₀ cycloalkylcarbonyl group, wherein the cycloalkyl is optionally substituted with 1 to 3 groups independently selected from the group consisting of a C₁₋₆ alkyl group, a halogen atom, a hydroxyl group, and a C₁₋₆ alkoxy group.

R^{4A}, R^{4B}, R^{4C}, and R^{4D} are still more preferably the same or different, each independently

(1) a hydrogen atom,
(2) a halogen atom,
(3) a cyano group,
(4) a hydroxyl group,
(5) an amino group optionally substituted with a C₁₋₆ alkyl group, or
(6) a C₁₋₆ alkyl group.

R^{4A}, R^{4B}, R^{4C}, and R^{4D} are still more preferably the same or different, each independently

(1) a hydrogen atom, or
(2) a halogen atom,

R^{4A}, R^{4B}, R^{4C}, and R^{4D} are the most preferably a hydrogen atom.

X is preferably a halogen atom, more preferably a fluorine atom, a chlorine atom, a bromine atom, or an iodine atom, more preferably a chorine atom or a bromine atom, and most preferably a chlorine atom.

Examples of preferred compounds among compound represented by formula (3) or (5) include the following compounds. Thus, it is understood that tautomers of the following preferred compounds, stereoisomers thereof, mixtures and racemates thereof, optionally pharmaceutically acceptable salts thereof, and solvates thereof are also preferred in a preferred embodiment.

Compounds represented by formula (3) include the following (3A).

### (3A)

A compound or a pharmaceutically acceptable salt thereof,

or a solvate thereof, wherein

R^{1A} and R^{1B} are the same or different, each independently

(1) a hydrogen atom,
(2) an optionally substituted C₁₋₁₀ alkyl group, or
(3) an optionally substituted C₃₋₁₀ cycloalkyl group,
wherein R^{1A} and R^{1B} are not simultaneously a hydrogen atom, and

R^{4A}, R^{4B}, R^{4C}, and R^{4D} are the same or different, each independently

(1) a hydrogen atom,
(2) a halogen atom,
(3) a cyano group,
(4) a hydroxyl group,
(5) an optionally substituted amino group,
(6) an optionally substituted C₁₋₆ alkyl group,
(7) an optionally substituted C₃₋₁₀ cycloalkyl group,
(8) an optionally substituted C₆₋₁₀ aryl group,
(9) an optionally substituted 3- to 12-membered monocyclic or polycyclic heterocyclic group,
(10) an optionally substituted C₁₋₆ alkoxy group,
(11) an optionally substituted C₃₋₁₀ cycloalkoxy group,
(12) an optionally substituted C₆₋₁₀ aryloxy group,
(13) an optionally substituted 3- to 12-membered monocyclic or polycyclic heterocyclic oxy group,
(14) a carboxyl group,
(15) an optionally substituted C₁₋₆ alkylcarbonyl group,
(16) an optionally substituted C₃₋₁₀ cycloalkylcarbonyl group,
(17) an optionally substituted C₆₋₁₀ arylcarbonyl group,
(18) an optionally substituted 3- to 12-membered monocyclic or polycyclic heterocyclic carbonyl group,
(19) an optionally substituted C₁₋₆ alkoxycarbonyl group,
(20) an optionally substituted C₃₋₁₀ cycloalkoxycarbonyl group,
(21) an optionally substituted C₆₋₁₀ aryloxycarbonyl group,
(22) an optionally substituted 3- to 12-membered monocyclic or polycyclic heterocyclic oxycarbonyl group,
(23) an optionally substituted aminocarbonyl group,
(24) an optionally substituted C₁₋₆ alkylthio group,
(25) an optionally substituted C₃₋₁₀ cycloalkylthio group,
(26) an optionally substituted C₆₋₁₀ arylthio group,
(27) an optionally substituted 3- to 12-membered monocyclic or polycyclic heterocyclic thio group,
(28) a sulfinic acid group,
(29) an optionally substituted C₁₋₆ alkylsulfinyl group,
(30) an optionally substituted C₃₋₁₀ cycloalkylsulfinyl group,
(31) an optionally substituted C₆₋₁₀ arylsulfinyl group,
(32) an optionally substituted 3- to 12-membered monocyclic or polycyclic heterocyclic sulfinyl group,
(33) an optionally substituted aminosulfinyl group,
(34) a sulfonic acid group,
(35) an optionally substituted C₁₋₆ alkylsulfonyl group,
(36) an optionally substituted C₃₋₁₀ cycloalkylsulfonyl group,
(37) an optionally substituted C₆₋₁₀ arylsulfonyl group,
(38) an optionally substituted 3- to 12-membered monocyclic or polycyclic heterocyclic sulfonyl group, or
(39) an optionally substituted aminosulfonyl group.

A preferred embodiment of compounds represented by formula (3) includes the following (3B):

### (3B)

A compound or a pharmaceutically acceptable salt thereof,

or a solvate thereof, wherein

R^{1A} and R^{1B} are the same or different, each independently

(1) a hydrogen atom,
(2) a C₁₋₆ alkyl group, wherein the alkyl group is optionally substituted with 1 to 3 groups independently selected from the group consisting of a phenyl group, a halogen atom, a hydroxyl group, and a C₁₋₆ alkoxy group, or
(3) a C₃₋₁₀ cycloalkyl group, wherein the cycloalkyl group is optionally substituted with 1 to 3 groups independently selected from the group consisting of a C₁₋₆ alkyl group, a phenyl group, a halogen atom, a hydroxyl group, and a C₁₋₆ alkoxy group,

wherein R^{1A} and R^{1B} are not simultaneously a hydrogen atom, and

R^{4A}, R^{4B}, R^{4C}, and R^{4D} are preferably the same or different, each independently

(1) a hydrogen atom,
(2) a halogen atom,
(3) a cyano group,
(4) a hydroxyl group,
(5) an amino group optionally substituted with a C₁₋₆ alkyl group,
(6) a C₁₋₆ alkyl group, wherein the alkyl group is optionally substituted with 1 to 3 groups independently selected from the group consisting of a phenyl group, a halogen atom, a hydroxyl group, and a C₁₋₆ alkoxy group,
(7) a C₃₋₁₀ cycloalkyl group, wherein the cycloalkyl group is optionally substituted with 1 to 3 groups independently selected from the group consisting of a C₁₋₆ alkyl group, a phenyl group, a halogen atom, a hydroxyl group, and a C₁₋₆ alkoxy group,
(8) a C₆₋₁₀ aryl group, wherein the aryl group is optionally substituted with 1 to 3 groups independently selected from the group consisting of a C₁₋₆ alkyl group, a phenyl group, a halogen atom, a hydroxyl group, and a C₁₋₆ alkoxy group,
(9) a 3- to 12-membered monocyclic or polycyclic heterocyclic group, wherein the heterocyclic group is optionally substituted with 1 to 3 groups independently selected from the group consisting of a C₁₋₆ alkyl group, a phenyl group, a halogen atom, a hydroxyl group, and a C₁₋₆ alkoxy group,
(10) a C₁₋₆ alkoxy group, wherein the alkoxy group is optionally substituted with 1 to 3 groups independently selected from the group consisting of a phenyl group, a halogen atom, a hydroxyl group, and a C₁₋₆ alkoxy group,
(11) a C₃₋₁₀ cycloalkoxy group, wherein the cycloalkoxy group is optionally substituted with 1 to 3 groups independently selected from the group consisting of a C₁₋₆ alkyl group, a phenyl group, a halogen atom, a hydroxyl group, and a C₁₋₆ alkoxy group,
(12) a C₆₋₁₀ aryloxy group, wherein the aryl group is optionally substituted with 1 to 3 groups independently selected from the group consisting of a C₁₋₆ alkyl group, a phenyl group, a halogen atom, a hydroxyl group, and a C₁₋₆ alkoxy group,
(13) a 3- to 12-membered monocyclic or polycyclic heterocyclic oxy group, wherein the heterocycle is optionally substituted with 1 to 3 groups independently selected from the group consisting of a C₁₋₆ alkyl group, a phenyl group, a halogen atom, a hydroxyl group, and a C₁₋₆ alkoxy group,
(14) a carboxyl group,
(15) a C₁₋₆ alkylcarbonyl group, wherein the alkyl is optionally substituted with 1 to 3 groups independently selected from the group consisting of a phenyl group, a halogen atom, a hydroxyl group, and a C₁₋₆ alkoxy group,
(16) a C₃₋₁₀ cycloalkylcarbonyl group, wherein the cycloalkyl is optionally substituted with 1 to 3 groups independently selected from the group consisting of a C₁₋₆ alkyl group, a phenyl group, a halogen atom, a hydroxyl group, and a C₁₋₆ alkoxy group,
(17) a C₆₋₁₀ arylcarbonyl group, wherein the aryl is optionally substituted with 1 to 3 groups independently selected from the group consisting of a C₁₋₆ alkyl group, a phenyl group, a halogen atom, a hydroxyl group, and a C₁₋₆ alkoxy group,
(18) a 3- to 12-membered monocyclic or polycyclic heterocyclic carbonyl group, wherein the heterocycle is optionally substituted with 1 to 3 groups independently selected from the group consisting of a C₁₋₆ alkyl group, a phenyl group, a halogen atom, a hydroxyl group, and a C₁₋₆ alkoxy group,
(19) a C₁₋₆ alkoxycarbonyl group, wherein the alkoxy is optionally substituted with 1 to 3 groups independently selected from the group consisting of a phenyl group, a halogen atom, a hydroxyl group, and a C₁₋₆ alkoxy group,
(20) a C₃₋₁₀ cycloalkoxycarbonyl group, wherein the cycloalkoxy is optionally substituted with 1 to 3 groups independently selected from the group consisting of a C₁₋₆ alkyl group, a phenyl group, a halogen atom, a hydroxyl group, and a C₁₋₆ alkoxy group,
(21) a C₆₋₁₀ aryloxycarbonyl group, wherein the aryl is optionally substituted with 1 to 3 groups independently selected from the group consisting of a C₁₋₆ alkyl group, a phenyl group, a halogen atom, a hydroxyl group, and a C₁₋₆ alkoxy group,
(22) a 3- to 12-membered monocyclic or polycyclic heterocyclic oxycarbonyl group, wherein the heterocycle is optionally substituted with 1 to 3 groups independently selected from the group consisting of a C₁₋₆ alkyl group, a phenyl group, a halogen atom, a hydroxyl group, and a C₁₋₆ alkoxy group, or
(23) an aminocarbonyl group optionally substituted with a C₁₋₆ alkyl group.

A preferred embodiment of compounds represented by formula (3) includes the following (3C):

### (3C)

A compound or a pharmaceutically acceptable salt thereof,

or a solvate thereof, wherein

R^{1A} and R^{1B} are the same or different, each independently

(1) a hydrogen atom,
(2) a C₁₋₆ alkyl group, or
(3) a C₃₋₁₀ cycloalkyl group,

wherein R^{1A} and R^{1B} are not simultaneously a hydrogen atom, and

R^{4A}, R^{4B}, R^{4C}, and R^{4D} are the same or different, each independently

(1) a hydrogen atom,
(2) a halogen atom,
(3) a cyano group,
(4) a hydroxyl group,
(5) an amino group optionally substituted with a C₁₋₆ alkyl group,
(6) a C₁₋₆ alkyl group, wherein the alkyl group is optionally substituted with 1 to 3 groups independently selected from the group consisting of a halogen atom, a hydroxyl group, and a C₁₋₆ alkoxy group,
(7) a C₃₋₁₀ cycloalkyl group, wherein the cycloalkyl group is optionally substituted with 1 to 3 groups independently selected from the group consisting of a C₁₋₆ alkyl group, a halogen atom, a hydroxyl group, and a C₁₋₆ alkoxy group,
(8) a C₆₋₁₀ aryl group, wherein the aryl group is optionally substituted with 1 to 3 groups independently selected from the group consisting of a C₁₋₆ alkyl group, a halogen atom, a hydroxyl group, and a C₁₋₆ alkoxy group,
(9) a 3- to 12-membered monocyclic or polycyclic heterocyclic group, wherein the heterocyclic group is optionally substituted with 1 to 3 groups independently selected from the group consisting of a C₁₋₆ alkyl group, a halogen atom, a hydroxyl group, and a C₁₋₆ alkoxy group,
(10) a C₁₋₆ alkoxy group, wherein the alkoxy group is optionally substituted with 1 to 3 groups independently selected from the group consisting of a halogen atom, a hydroxyl group, and a C₁₋₆ alkoxy group,
(11) a C₃₋₁₀ cycloalkoxy group, wherein the cycloalkoxy group is optionally substituted with 1 to 3 groups independently selected from the group consisting of a C₁₋₆ alkyl group, a halogen atom, a hydroxyl group, and a C₁₋₆ alkoxy group,
(12) a C₆₋₁₀ aryloxy group, wherein the aryl group is optionally substituted with 1 to 3 groups independently selected from the group consisting of a C₁₋₆ alkyl group, a halogen atom, a hydroxyl group, and a C₁₋₆ alkoxy group,
(13) a 3- to 12-membered monocyclic or polycyclic heterocyclic oxy group, wherein the heterocycle is optionally substituted with 1 to 3 groups independently selected from the group consisting of a C₁₋₆ alkyl group, a halogen atom, a hydroxyl group, and a C₁₋₆ alkoxy group,
(14) a carboxyl group,
(15) a C₁₋₆ alkylcarbonyl group, wherein the alkyl is optionally substituted with 1 to 3 groups independently selected from the group consisting of a halogen atom, a hydroxyl group, and a C₁₋₆ alkoxy group, or
(16) a C₃₋₁₀ cycloalkylcarbonyl group, wherein the cycloalkyl is optionally substituted with 1 to 3 groups independently selected from the group consisting of a C₁₋₆ alkyl group, a halogen atom, a hydroxyl group, and a C₁₋₆ alkoxy group.

A more preferred embodiment of compounds represented by formula (3) includes the following (3D):

### (3D)

A compound or a pharmaceutically acceptable salt thereof,

or a solvate thereof, wherein

R^{1A} and R^{1B} are the same or different, each independently a hydrogen atom or a C₁_₃ alkyl group, wherein R^{1A} and R^{1B} are not simultaneously a hydrogen atom, and

R^{4A}, R^{4B}, R^{4C}, and R^{4D} are the same or different, each independently

(1) a hydrogen atom,
(2) a halogen atom,
(3) a cyano group,
(4) a hydroxyl group,
(5) an amino group optionally substituted with a C₁₋₆ alkyl group, or
(6) a C₁₋₆ alkyl group.

A still more preferred embodiment of compounds represented by formula (3) includes the following (3E):

### (3E)

A compound or a pharmaceutically acceptable salt thereof, or a solvate thereof, wherein

R^{1A} and R^{1B} are each independently a methyl group, an ethyl group, a propyl group, or an isopropyl group, and

R^{4A}, R^{4B}, R^{4C}, and R^{4D} are the same or different, each independently a hydrogen atom or a halogen atom.

The most preferred embodiment of compounds represented by formula (3) includes the following (3G):

### (3G)

A compound or a pharmaceutically acceptable salt thereof, or a solvate thereof, wherein

R^{1A} and R^{1B} are methyl groups, and

R^{4A}, R^{4B}, R^{4C}, and R^{4D} are hydrogen atoms.

Compounds represented by formula (5) include the following (5A):

### (5A)

A compound or a pharmaceutically acceptable salt thereof, or a solvate thereof, wherein

R² is

(1) a hydrogen atom,
(2) an optionally substituted C₁₋₁₀ alkyl group,
(3) an optionally substituted C₃₋₁₀ cycloalkyl group,
(4) an optionally substituted C₆₋₁₀ aryl group, or
(5) an optionally substituted 3- to 12-membered monocyclic or polycyclic heterocyclic group,

R³ is

(1) a hydrogen atom,
(2) an optionally substituted C₁₋₁₀ alkyl group,
(3) an optionally substituted C₃₋₁₀ cycloalkyl group,
(4) an optionally substituted C₆₋₁₀ aryl group, or
(5) an optionally substituted 3- to 12-membered monocyclic or polycyclic heterocyclic group, and

R^{4A}, R^{4B}, R^{4C}, and R^{4D} are the same or different, each independently

(1) a hydrogen atom,
(2) a halogen atom,
(3) a cyano group,
(4) a hydroxyl group,
(5) an optionally substituted amino group,
(6) an optionally substituted C₁₋₆ alkyl group,
(7) an optionally substituted C₃₋₁₀ cycloalkyl group,
(8) an optionally substituted C₆₋₁₀ aryl group,
(9) an optionally substituted 3- to 12-membered monocyclic or polycyclic heterocyclic group,
(10) an optionally substituted C₁₋₆ alkoxy group,
(11) an optionally substituted C₃₋₁₀ cycloalkoxy group,
(12) an optionally substituted C₆₋₁₀ aryloxy group,
(13) an optionally substituted 3- to 12-membered monocyclic or polycyclic heterocyclic oxy group,
(14) a carboxyl group,
(15) an optionally substituted C₁₋₆ alkylcarbonyl group,
(16) an optionally substituted C₃₋₁₀ cycloalkylcarbonyl group,
(17) an optionally substituted C₆₋₁₀ arylcarbonyl group,
(18) an optionally substituted 3- to 12-membered monocyclic or polycyclic heterocyclic carbonyl group,
(19) an optionally substituted C₁₋₆ alkoxycarbonyl group,
(20) an optionally substituted C₃₋₁₀ cycloalkoxycarbonyl group,
(21) an optionally substituted C₆₋₁₀ aryloxycarbonyl group,
(22) an optionally substituted 3- to 12-membered monocyclic or polycyclic heterocyclic oxycarbonyl group,
(23) an optionally substituted aminocarbonyl group,
(24) an optionally substituted C₁₋₆ alkylthio group,
(25) an optionally substituted C₃₋₁₀ cycloalkylthio group,
(26) an optionally substituted C₆₋₁₀ arylthio group,
(27) an optionally substituted 3- to 12-membered monocyclic or polycyclic heterocyclic thio group,
(28) a sulfinic acid group,
(29) an optionally substituted C₁₋₆ alkylsulfinyl group,
(30) an optionally substituted C₃₋₁₀ cycloalkylsulfinyl group,
(31) an optionally substituted C₆₋₁₀ arylsulfinyl group,
(32) an optionally substituted 3- to 12-membered monocyclic or polycyclic heterocyclic sulfinyl group,
(33) an optionally substituted aminosulfinyl group,
(34) a sulfonic acid group,
(35) an optionally substituted C₁₋₆ alkylsulfonyl group,
(36) an optionally substituted C₃₋₁₀ cycloalkylsulfonyl group,
(37) an optionally substituted C₆₋₁₀ arylsulfonyl group,
(38) an optionally substituted 3- to 12-membered monocyclic or polycyclic heterocyclic sulfonyl group, or
(39) an optionally substituted aminosulfonyl group.

Compounds represented by formula (5) include the following (5B):

### (5B)

A compound or a pharmaceutically acceptable salt thereof, or a solvate thereof, wherein

R² is a C₁₋₁₀ alkyl group optionally substituted with 1 to 3 groups independently selected from the group consisting of a phenyl group, a halogen atom, a hydroxyl group, and a C₁₋₆ alkoxy group,

R³ is

(1) a hydrogen atom, or
(2) a C₁₋₁₀ alkyl group, a C₃₋₁₀ cycloalkyl group, a C₆₋₁₀ aryl group, a 3- to 12-membered monocyclic or polycyclic heterocyclic group (wherein the alkyl group is optionally substituted with 1 to 3 groups independently selected from the group consisting of a phenyl group, a halogen atom, a hydroxyl group, and a C₁₋₆ alkoxy group, and the cycloalkyl group, aryl group, or heterocyclic group is optionally substituted with 1 to 3 groups independently selected from the group consisting of a C₁₋₆ alkyl group, a phenyl group, a halogen atom, a hydroxyl group, and a C₁₋₆ alkoxy group, and R^{4A}, R^{4B}, R^{4C}, and R^{4D} are the same or different, each independently
(1) a hydrogen atom,
(2) a halogen atom,
(3) a cyano group,
(4) a hydroxyl group,
(5) an amino group optionally substituted with a C₁₋₆ alkyl group,
(6) a C₁₋₆ alkyl group, wherein the alkyl group is optionally substituted with 1 to 3 groups independently selected from the group consisting of a phenyl group, a halogen atom, a hydroxyl group, and a C₁₋₆ alkoxy group,
(7) a C₃₋₁₀ cycloalkyl group, wherein the cycloalkyl group is optionally substituted with 1 to 3 groups independently selected from the group consisting of a C₁₋₆ alkyl group, a phenyl group, a halogen atom, a hydroxyl group, and a C₁₋₆ alkoxy group,
(8) a C₆₋₁₀ aryl group, wherein the aryl group is optionally substituted with 1 to 3 groups independently selected from the group consisting of a C₁₋₆ alkyl group, a phenyl group, a halogen atom, a hydroxyl group, and a C₁₋₆ alkoxy group,
(9) a 3- to 12-membered monocyclic or polycyclic heterocyclic group, wherein the heterocyclic group is optionally substituted with 1 to 3 groups independently selected from the group consisting of a C₁₋₆ alkyl group, a phenyl group, a halogen atom, a hydroxyl group, and a C₁₋₆ alkoxy group,
(10) a C₁₋₆ alkoxy group, wherein the alkoxy group is optionally substituted with 1 to 3 groups independently selected from the group consisting of a phenyl group, a halogen atom, a hydroxyl group, and a C₁₋₆ alkoxy group,
(11) a C₃₋₁₀ cycloalkoxy group, wherein the cycloalkoxy group is optionally substituted with 1 to 3 groups independently selected from the group consisting of a C₁₋₆ alkyl group, a phenyl group, a halogen atom, a hydroxyl group, and a C₁₋₆ alkoxy group,
(12) a C₆₋₁₀ aryloxy group, wherein the aryl group is optionally substituted with 1 to 3 groups independently selected from the group consisting of a C₁₋₆ alkyl group, a phenyl group, a halogen atom, a hydroxyl group, and a C₁₋₆ alkoxy group,
(13) a 3- to 12-membered monocyclic or polycyclic heterocyclic oxy group, wherein the heterocycle is optionally substituted with 1 to 3 groups independently selected from the group consisting of a C₁₋₆ alkyl group, a phenyl group, a halogen atom, a hydroxyl group, and a C₁₋₆ alkoxy group,
(14) a carboxyl group,
(15) a C₁₋₆ alkylcarbonyl group, wherein the alkyl is optionally substituted with 1 to 3 groups independently selected from the group consisting of a phenyl group, a halogen atom, a hydroxyl group, and a C₁₋₆ alkoxy group,
(16) a C₃₋₁₀ cycloalkylcarbonyl group, wherein the cycloalkyl is optionally substituted with 1 to 3 groups independently selected from the group consisting of a C₁₋₆ alkyl group, a phenyl group, a halogen atom, a hydroxyl group, and a C₁₋₆ alkoxy group,
(17) a C₆₋₁₀ arylcarbonyl group, wherein the aryl is optionally substituted with 1 to 3 groups independently selected from the group consisting of a C₁₋₆ alkyl group, a phenyl group, a halogen atom, a hydroxyl group, and a C₁₋₆ alkoxy group,
(18) a 3- to 12-membered monocyclic or polycyclic heterocyclic carbonyl group, wherein the heterocycle is optionally substituted with 1 to 3 groups independently selected from the group consisting of a C₁₋₆ alkyl group, a phenyl group, a halogen atom, a hydroxyl group, and a C₁₋₆ alkoxy group,
(19) a C₁₋₆ alkoxycarbonyl group, wherein the alkoxy is optionally substituted with 1 to 3 groups independently selected from the group consisting of a phenyl group, a halogen atom, a hydroxyl group, and a C₁₋₆ alkoxy group,
(20) a C₃₋₁₀ cycloalkoxycarbonyl group, wherein the cycloalkoxy is optionally substituted with 1 to 3 groups independently selected from the group consisting of a C₁₋₆ alkyl group, a phenyl group, a halogen atom, a hydroxyl group, and a C₁₋₆ alkoxy group,
(21) a C₆₋₁₀ aryloxycarbonyl group, wherein the aryl is optionally substituted with 1 to 3 groups independently selected from the group consisting of a C₁₋₆ alkyl group, a phenyl group, a halogen atom, a hydroxyl group, and a C₁₋₆ alkoxy group,
(22) a 3- to 12-membered monocyclic or polycyclic heterocyclic oxycarbonyl group, wherein the heterocycle is optionally substituted with 1 to 3 groups independently selected from the group consisting of a C₁₋₆ alkyl group, a phenyl group, a halogen atom, a hydroxyl group, and a C₁₋₆ alkoxy group, or
(23) an aminocarbonyl group optionally substituted with a C₁₋₆ alkyl group.

Compounds represented by formula (5) include the following (5C):

### (5C)

A compound or a pharmaceutically acceptable salt thereof, or a solvate thereof, wherein

R² is a C₁₋₆ alkyl group optionally substituted with 1 to 3 groups independently selected from the group consisting of a hydroxyl group and a C₁₋₆ alkoxy group,

R³ is a C₁₋₆ alkyl group optionally substituted with 1 to 3 groups independently selected from the group consisting of a halogen atom, a hydroxyl group, and a C₁₋₆ alkoxy group, and

R^{4A}, R^{4B}, R^{4C}, and R^{4D} are the same or different, each independently

(1) a hydrogen atom,
(2) a halogen atom,
(3) a cyano group,
(4) a hydroxyl group,
(5) an amino group optionally substituted with a C₁₋₆ alkyl group,
(6) a C₁₋₆ alkyl group, wherein the alkyl group is optionally substituted with 1 to 3 groups independently selected from the group consisting of a halogen atom, a hydroxyl group, and a C₁₋₆ alkoxy group,
(7) a C₃₋₁₀ cycloalkyl group, wherein the cycloalkyl group is optionally substituted with 1 to 3 groups independently selected from the group consisting of a C₁₋₆ alkyl group, a halogen atom, a hydroxyl group, and a C₁₋₆ alkoxy group,
(8) a C₆₋₁₀ aryl group, wherein the aryl group is optionally substituted with 1 to 3 groups independently selected from the group consisting of a C₁₋₆ alkyl group, a halogen atom, a hydroxyl group, and a C₁₋₆ alkoxy group,
(9) a 3- to 12-membered monocyclic or polycyclic heterocyclic group, wherein the heterocyclic group is optionally substituted with 1 to 3 groups independently selected from the group consisting of a C₁₋₆ alkyl group, a halogen atom, a hydroxyl group, and a C₁₋₆ alkoxy group,
(10) a C₁₋₆ alkoxy group, wherein the alkoxy group is optionally substituted with 1 to 3 groups independently selected from the group consisting of a halogen atom, a hydroxyl group, and a C₁₋₆ alkoxy group,
(11) a C₃₋₁₀ cycloalkoxy group, wherein the cycloalkoxy group is optionally substituted with 1 to 3 groups independently selected from the group consisting of a C₁₋₆ alkyl group, a halogen atom, a hydroxyl group, and a C₁₋₆ alkoxy group,
(12) a C₆₋₁₀ aryloxy group, wherein the aryl group is optionally substituted with 1 to 3 groups independently selected from the group consisting of a C₁₋₆ alkyl group, a halogen atom, a hydroxyl group, and a C₁₋₆ alkoxy group,
(13) a 3- to 12-membered monocyclic or polycyclic heterocyclic oxy group, wherein the heterocycle is optionally substituted with 1 to 3 groups independently selected from the group consisting of a C₁₋₆ alkyl group, a halogen atom, a hydroxyl group, and a C₁₋₆ alkoxy group,
(14) a carboxyl group,
(15) a C₁₋₆ alkylcarbonyl group, wherein the alkyl is optionally substituted with 1 to 3 groups independently selected from the group consisting of a halogen atom, a hydroxyl group, and a C₁₋₆ alkoxy group, or
(16) a C₃₋₁₀ cycloalkylcarbonyl group, wherein the cycloalkyl is optionally substituted with 1 to 3 groups independently selected from the group consisting of a C₁₋₆ alkyl group, a halogen atom, a hydroxyl group, and a C₁₋₆ alkoxy group.

Compounds represented by formula (5) include the following (5D):

### (5D)

A compound or a pharmaceutically acceptable salt thereof, or a solvate thereof, wherein

R² is a C₁₋₃ alkyl group,

R³ is a C₁₋₆ alkyl group, and

R^{4A}, R^{4B}, R^{4C}, and R^{4D} are the same or different, each independently

(1) a hydrogen atom,
(2) a halogen atom,
(3) a cyano group,
(4) a hydroxyl group,
(5) an amino group optionally substituted with a C₁₋₆ alkyl group, or
(6) a C₁₋₆ alkyl group.

Compounds represented by formula (5) include the following (5E):

### (5E)

A compound or a pharmaceutically acceptable salt thereof, or a solvate thereof, wherein

R² is a methyl group, an ethyl group, a propyl group, or an isopropyl group,

R³ is a methyl group, an ethyl group, a propyl group, an isopropyl group, or a butyl group, and

R^{4A}, R^{4B}, R^{4C}, and R^{4D} are the same or different, each independently

(1) a hydrogen atom, or
(2) a halogen atom.

Compounds represented by formula (5) include the following (5F):

### (5F)

A compound or a pharmaceutically acceptable salt thereof, or a solvate thereof, wherein

R² is a methyl group,

R³ is a methyl group, and

R^{4A}, R^{4B}, R^{4C}, and R^{4D} are hydrogen atoms.

Compounds represented by formula (6) include the following (6A):

### (6A)

A compound or a pharmaceutically acceptable salt thereof, or a solvate thereof, wherein

R² is

(1) a hydrogen atom,
(2) an optionally substituted C₁₋₁₀ alkyl group,
(3) an optionally substituted C₃₋₁₀ cycloalkyl group,
(4) an optionally substituted C₆₋₁₀ aryl group, or
(5) an optionally substituted 3- to 12-membered monocyclic or polycyclic heterocyclic group, and

R^{4A}, R^{4B}, R^{4C}, and R^{4D} are the same or different, each independently

(1) a hydrogen atom,
(2) a halogen atom,
(3) a cyano group,
(4) a hydroxyl group,
(5) an optionally substituted amino group,
(6) an optionally substituted C₁₋₆ alkyl group,
(7) an optionally substituted C₃₋₁₀ cycloalkyl group,
(8) an optionally substituted C₆₋₁₀ aryl group,
(9) an optionally substituted 3- to 12-membered monocyclic or polycyclic heterocyclic group,
(10) an optionally substituted C₁₋₆ alkoxy group,
(11) an optionally substituted C₃₋₁₀ cycloalkoxy group,
(12) an optionally substituted C₆₋₁₀ aryloxy group,
(13) an optionally substituted 3- to 12-membered monocyclic or polycyclic heterocyclic oxy group,
(14) a carboxyl group,
(15) an optionally substituted C₁₋₆ alkylcarbonyl group,
(16) an optionally substituted C₃₋₁₀ cycloalkylcarbonyl group,
(17) an optionally substituted C₆₋₁₀ arylcarbonyl group,
(18) an optionally substituted 3- to 12-membered monocyclic or polycyclic heterocyclic carbonyl group,
(19) an optionally substituted C₁₋₆ alkoxycarbonyl group,
(20) an optionally substituted C₃₋₁₀ cycloalkoxycarbonyl group,
(21) an optionally substituted C₆₋₁₀ aryloxycarbonyl group,
(22) an optionally substituted 3- to 12-membered monocyclic or polycyclic heterocyclic oxycarbonyl group,
(23) an optionally substituted aminocarbonyl group,
(24) an optionally substituted C₁₋₆ alkylthio group,
(25) an optionally substituted C₃₋₁₀ cycloalkylthio group,
(26) an optionally substituted C₆₋₁₀ arylthio group,
(27) an optionally substituted 3- to 12-membered monocyclic or polycyclic heterocyclic thio group,
(28) a sulfinic acid group,
(29) an optionally substituted C₁₋₆ alkylsulfinyl group,
(30) an optionally substituted C₃₋₁₀ cycloalkylsulfinyl group,
(31) an optionally substituted C₆₋₁₀ arylsulfinyl group,
(32) an optionally substituted 3- to 12-membered monocyclic or polycyclic heterocyclic sulfinyl group,
(33) an optionally substituted aminosulfinyl group,
(34) a sulfonic acid group,
(35) an optionally substituted C₁₋₆ alkylsulfonyl group,
(36) an optionally substituted C₃₋₁₀ cycloalkylsulfonyl group,
(37) an optionally substituted C₆₋₁₀ arylsulfonyl group,
(38) an optionally substituted 3- to 12-membered monocyclic or polycyclic heterocyclic sulfonyl group, or
(39) an optionally substituted aminosulfonyl group.

Compounds represented by formula (6) include the following (6B):

### (6B)

A compound or a pharmaceutically acceptable salt thereof, or a solvate thereof, wherein

R² is a C₁₋₁₀ alkyl group optionally substituted with 1 to 3 groups independently selected from the group consisting of a phenyl group, a halogen atom, a hydroxyl group, and a C₁₋₆ alkoxy group, and

R^{4A}, R^{4B}, R^{4C}, and R^{4D} are the same or different, each independently

(1) a hydrogen atom,
(2) a halogen atom,
(3) a cyano group,
(4) a hydroxyl group,
(5) an amino group optionally substituted with a C₁₋₆ alkyl group,
(6) a C₁₋₆ alkyl group, wherein the alkyl group is optionally substituted with 1 to 3 groups independently selected from the group consisting of a phenyl group, a halogen atom, a hydroxyl group, and a C₁₋₆ alkoxy group,
(7) a C₃₋₁₀ cycloalkyl group, wherein the cycloalkyl group is optionally substituted with 1 to 3 groups independently selected from the group consisting of a C₁₋₆ alkyl group, a phenyl group, a halogen atom, a hydroxyl group, and a C₁₋₆ alkoxy group,
(8) a C₆₋₁₀ aryl group, wherein the aryl group is optionally substituted with 1 to 3 groups independently selected from the group consisting of a C₁₋₆ alkyl group, a phenyl group, a halogen atom, a hydroxyl group, and a C₁₋₆ alkoxy group,
(9) a 3- to 12-membered monocyclic or polycyclic heterocyclic group, wherein the heterocyclic group is optionally substituted with 1 to 3 groups independently selected from the group consisting of a C₁₋₆ alkyl group, a phenyl group, a halogen atom, a hydroxyl group, and a C₁₋₆ alkoxy group,
(10) a C₁₋₆ alkoxy group, wherein the alkoxy group is optionally substituted with 1 to 3 groups independently selected from the group consisting of a phenyl group, a halogen atom, a hydroxyl group, and a C₁₋₆ alkoxy group,
(11) a C₃₋₁₀ cycloalkoxy group, wherein the cycloalkoxy group is optionally substituted with 1 to 3 groups independently selected from the group consisting of a C₁₋₆ alkyl group, a phenyl group, a halogen atom, a hydroxyl group, and a C₁₋₆ alkoxy group,
(12) a C₆₋₁₀ aryloxy group, wherein the aryl group is optionally substituted with 1 to 3 groups independently selected from the group consisting of a C₁₋₆ alkyl group, a phenyl group, a halogen atom, a hydroxyl group, and a C₁₋₆ alkoxy group,
(13) a 3- to 12-membered monocyclic or polycyclic heterocyclic oxy group, wherein the heterocycle is optionally substituted with 1 to 3 groups independently selected from the group consisting of a C₁₋₆ alkyl group, a phenyl group, a halogen atom, a hydroxyl group, and a C₁₋₆ alkoxy group,
(14) a carboxyl group,
(15) a C₁₋₆ alkylcarbonyl group, wherein the alkyl is optionally substituted with 1 to 3 groups independently selected from the group consisting of a phenyl group, a halogen atom, a hydroxyl group, and a C₁₋₆ alkoxy group,
(16) a C₃₋₁₀ cycloalkylcarbonyl group, wherein the cycloalkyl is optionally substituted with 1 to 3 groups independently selected from the group consisting of a C₁₋₆ alkyl group, a phenyl group, a halogen atom, a hydroxyl group, and a C₁₋₆ alkoxy group,
(17) a C₆₋₁₀ arylcarbonyl group, wherein the aryl is optionally substituted with 1 to 3 groups independently selected from the group consisting of a C₁₋₆ alkyl group, a phenyl group, a halogen atom, a hydroxyl group, and a C₁₋₆ alkoxy group,
(18) a 3- to 12-membered monocyclic or polycyclic heterocyclic carbonyl group, wherein the heterocycle is optionally substituted with 1 to 3 groups independently selected from the group consisting of a C₁₋₆ alkyl group, a phenyl group, a halogen atom, a hydroxyl group, and a C₁₋₆ alkoxy group,
(19) a C₁₋₆ alkoxycarbonyl group, wherein the alkoxy is optionally substituted with 1 to 3 groups independently selected from the group consisting of a phenyl group, a halogen atom, a hydroxyl group, and a C₁₋₆ alkoxy group,
(20) a C₃₋₁₀ cycloalkoxycarbonyl group, wherein the cycloalkoxy is optionally substituted with 1 to 3 groups independently selected from the group consisting of a C₁₋₆ alkyl group, a phenyl group, a halogen atom, a hydroxyl group, and a C₁₋₆ alkoxy group,
(21) a C₆₋₁₀ aryloxycarbonyl group, wherein the aryl is optionally substituted with 1 to 3 groups independently selected from the group consisting of a C₁₋₆ alkyl group, a phenyl group, a halogen atom, a hydroxyl group, and a C₁₋₆ alkoxy group,
(22) a 3- to 12-membered monocyclic or polycyclic heterocyclic oxycarbonyl group, wherein the heterocycle is optionally substituted with 1 to 3 groups independently selected from the group consisting of a C₁₋₆ alkyl group, a phenyl group, a halogen atom, a hydroxyl group, and a C₁₋₆ alkoxy group, or
(23) an aminocarbonyl group optionally substituted with a C₁₋₆ alkyl group.

Compounds represented by formula (6) include the following (6C):

### (6C)

A compound or a pharmaceutically acceptable salt thereof, or a solvate thereof, wherein

R² is a C₁₋₆ alkyl group optionally substituted with 1 to 3 groups independently selected from the group consisting of a hydroxyl group and a C₁₋₆ alkoxy group, and

R^{4A}, R^{4B}, R^{4C}, and R^{4D} are the same or different, each independently

(1) a hydrogen atom,
(2) a halogen atom,
(3) a cyano group,
(4) a hydroxyl group,
(5) an amino group optionally substituted with a C₁₋₆ alkyl group,
(6) a C₁₋₆ alkyl group, wherein the alkyl group is optionally substituted with 1 to 3 groups independently selected from the group consisting of a halogen atom, a hydroxyl group, and a C₁₋₆ alkoxy group,
(7) a C₃₋₁₀ cycloalkyl group, wherein the cycloalkyl group is optionally substituted with 1 to 3 groups independently selected from the group consisting of a C₁₋₆ alkyl group, a halogen atom, a hydroxyl group, and a C₁₋₆ alkoxy group,
(8) a C₆₋₁₀ aryl group, wherein the aryl group is optionally substituted with 1 to 3 groups independently selected from the group consisting of a C₁₋₆ alkyl group, a halogen atom, a hydroxyl group, and a C₁₋₆ alkoxy group,
(9) a 3- to 12-membered monocyclic or polycyclic heterocyclic group, wherein the heterocyclic group is optionally substituted with 1 to 3 groups independently selected from the group consisting of a C₁₋₆ alkyl group, a halogen atom, a hydroxyl group, and a C₁₋₆ alkoxy group,
(10) a C₁₋₆ alkoxy group, wherein the alkoxy group is optionally substituted with 1 to 3 groups independently selected from the group consisting of a halogen atom, a hydroxyl group, and a C₁₋₆ alkoxy group,
(11) a C₃₋₁₀ cycloalkoxy group, wherein the cycloalkoxy group is optionally substituted with 1 to 3 groups independently selected from the group consisting of a C₁₋₆ alkyl group, a halogen atom, a hydroxyl group, and a C₁₋₆ alkoxy group,
(12) a C₆₋₁₀ aryloxy group, wherein the aryl group is optionally substituted with 1 to 3 groups independently selected from the group consisting of a C₁₋₆ alkyl group, a halogen atom, a hydroxyl group, and a C₁₋₆ alkoxy group,
(13) a 3- to 12-membered monocyclic or polycyclic heterocyclic oxy group, wherein the heterocycle is optionally substituted with 1 to 3 groups independently selected from the group consisting of a C₁₋₆ alkyl group, a halogen atom, a hydroxyl group, and a C₁₋₆ alkoxy group,
(14) a carboxyl group,
(15) a C₁₋₆ alkylcarbonyl group, wherein the alkyl is optionally substituted with 1 to 3 groups independently selected from the group consisting of a halogen atom, a hydroxyl group, and a C₁₋₆ alkoxy group, or
(16) a C₃₋₁₀ cycloalkylcarbonyl group, wherein the cycloalkyl is optionally substituted with 1 to 3 groups independently selected from the group consisting of a C₁₋₆ alkyl group, a halogen atom, a hydroxyl group, and a C₁₋₆ alkoxy group.

Compounds represented by formula (6) include the following (6D):

### (6D)

A compound or a pharmaceutically acceptable salt thereof, or a solvate thereof, wherein

R² is a C₁_₃ alkyl group, and

R^{4A}, R^{4B}, R^{4C}, and R^{4D} are the same or different, each independently

(1) a hydrogen atom,
(2) a halogen atom,
(3) a cyano group,
(4) a hydroxyl group,
(5) an amino group optionally substituted with a C₁₋₆ alkyl group, or
(6) a C₁₋₆ alkyl group.

Compounds represented by formula (6) include the following (6E):

### (6E)

A compound or a pharmaceutically acceptable salt thereof, or a solvate thereof, wherein

R² is a methyl group, an ethyl group, a propyl group, or an isopropyl group, and

R^{4A}, R^{4B}, R^{4C}, and R^{4D} are the same or different, each independently

(1) a hydrogen atom, or
(2) a halogen atom.

Compounds represented by formula (6) include the following (6F):

### (6F)

A compound or a pharmaceutically acceptable salt thereof, or a solvate thereof, wherein

R² is a methyl group, and

R^{4A}, R^{4B}, R^{4C}, and R^{4D} are hydrogen atoms.

While the present disclosure is explained in more detail hereinafter with preferred embodiments, the technical scope of the present disclosure is not limited by the preferred embodiments. The present disclosure may also be altered to the extent that the altered invention remains within the scope of the present disclosure. It should be noted that compound names in the following preferred embodiments do not necessarily follow the IUPAC nomenclature.

The following abbreviations may be used to simplify the descriptions herein.
DMF: N,N-dimethylformamide,
Me: methyl group, and
DBU: 1,8-diazabicyclo[5.4.0]-7-undecene.

The manufacturing method of a compound of formula (3) or (5) in the present disclosure, or a tautomer thereof, a stereoisomer thereof, a mixture or racemate thereof, an optionally pharmaceutically acceptable salt thereof, or a solvate thereof (which are used as a manufacture intermediate of 2-alkylcarbonylnaphtho[2,3-b]furan-4,9-dione) is described below. Intermediates of 2-alkylcarbonylnaphtho[2,3-b]furan-4,9-dione and 2-alkylcarbonylnaphtho[2,3-b]furan-4,9-dione can be manufactured from a known compound by the following manufacturing method or a method corresponding thereto, or by appropriately combining synthesis methods that are well known to those skilled in the art.

While a compound obtained in each step can be used in a subsequent reaction directly as a reaction solution or as a composition, the compound can also be isolated from a reaction mixture by a conventional method, and readily purified by separation means such as recrystallization, distillation, or chromatography.

Unless specifically noted otherwise, each symbol of compounds in the following reactions is synonymous with the descriptions above.

The manufacturing method of the present disclosure is described hereinafter. The starting materials without a description hereinafter are commercially available, or can be manufactured by a method known to those skilled in the art or a method in accordance therewith.

Manufacturing Method 1 [Chemical Formula 83]

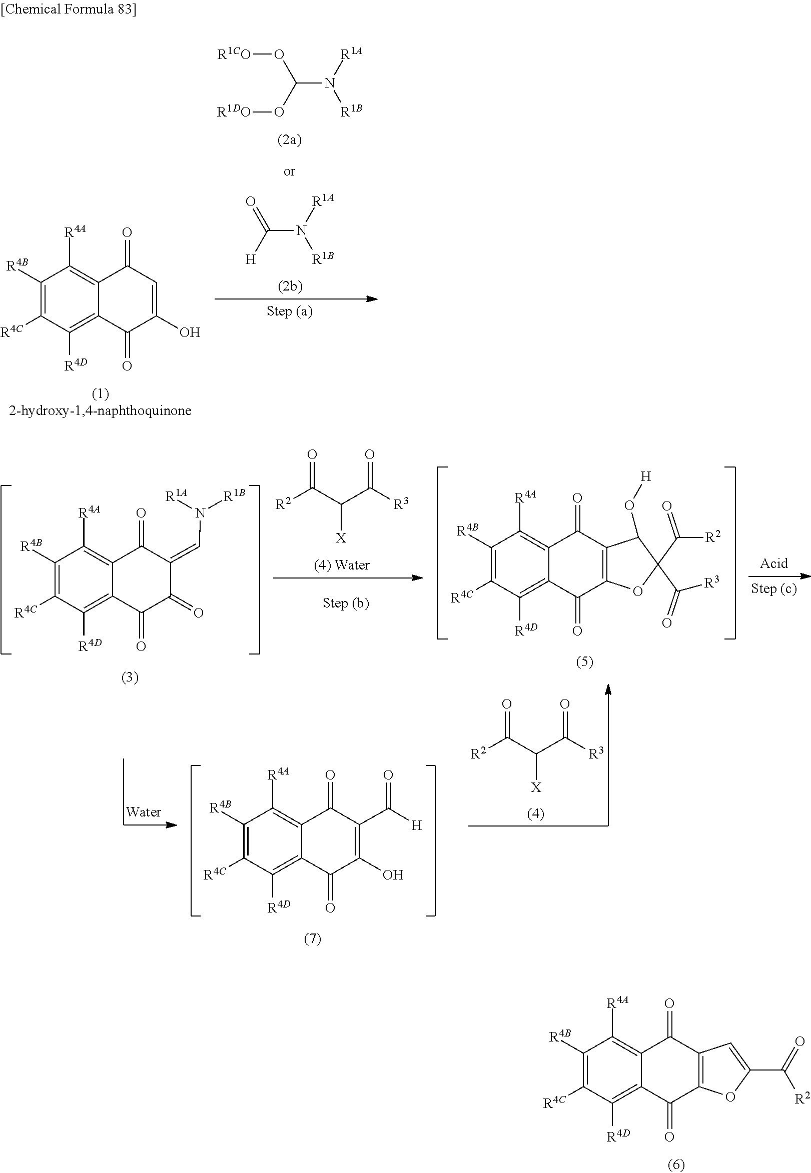

wherein R^{1A}, R^{1B}, R^{1C}, R^{1D}, R², R³, R^{4A}, R^{4B}, R^{4C}, R^{4D} are defined the same as item 1 and/or other items.

As described below, step (a) for constructing an enamine structure, step (b) for constructing a 2-acetyl-2-alkylcarbonyl-3-hydroxy-2,3-dihydronaphtho[2,3-b]furan-4,9-dione backbone, and step (c) are one of the most important features of the method of the present disclosure associated with Manufacturing Method 1. In particular, step (b) is the most important step. While steps (a), (b), and (c) are described hereinafter with a preferred embodiment, the present disclosure is not limited thereto.

Regarding Step (a):

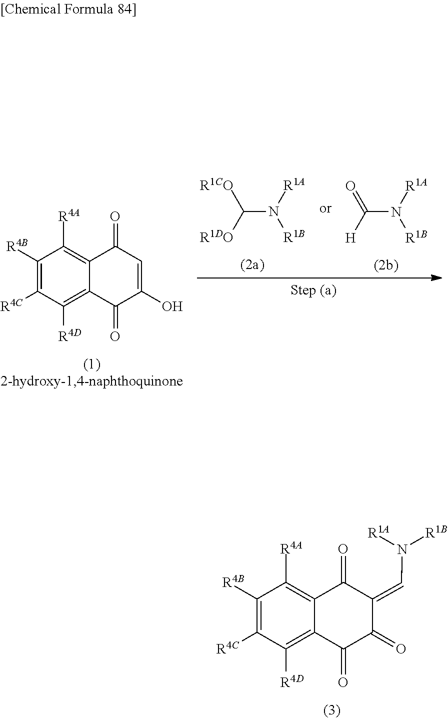

wherein R^{1A}, R^{1B}, R^{1C}, R^{1D}, R^{4A}, R^{4B}, R^{4A}, and R^{4B} are defined the same as item 1 and/or other items.

This is a step for obtaining a compound represented by formula (3) by reacting a compound represented by formula (2a) or formula (2b) with the compound 2-hydroxy-1,4-naphthoquinone (1) in a solvent.

The solvent used in this step is not particularly limited, as long as the boiling point is at or above the reaction temperature of this reaction. Examples thereof include ethylene glycol, N,N-dimethylacetamide, N,N-dimethylformamide, dimethyl sulfoxide, N-methyl-2-pyrrolidone, monochlorobenzene, and toluene. The solvent is preferably N,N-dimethylacetamide, N,N-dimethylformamide, N-methyl-2-pyrrolidone, or toluene, more preferably N,N-dimethylformamide or N-methyl-2-pyrrolidone, and still more preferably N,N-dimethylformamide.

Another preferred embodiment as a solvent used in this step includes an amide-based solvent.

The amount of solvent used in this step is generally 2-fold to 10-fold in weight, preferably 3-fold to 9-fold in weight, and more preferably 3-fold to 8-fold in weight with respect to 1 weight of 2-hydroxy-1,4-naphthoquinone.

The amount of compound represented by formula (2a) or formula (2b) used is generally 1.0 equivalent to 10.0 equivalent, preferably 1.0 equivalent to 5.0 equivalent, more preferably 1.0 equivalent to 2.0 equivalent, and most preferably 1.0 equivalent to 1.5 equivalent with respect to 1 equivalent of 2-hydroxy-1,4-naphthoquinone.

The reaction time is generally about 0.5 hours to 12 hours, and preferably 0.5 hours to 8 hours.

The reaction temperature is generally −30° C. to 120° C., preferably −10° C. to 130° C., more preferably −10° C. to 10° C., and still more preferably −5° C. to 5° C.

R^{1A}, R^{1B}, R^{1C}, R^{4D}, R^{4A}, R^{4B}, R^{4A}, and R^{4B} are preferably as described above.

Step (b) and Step (c)

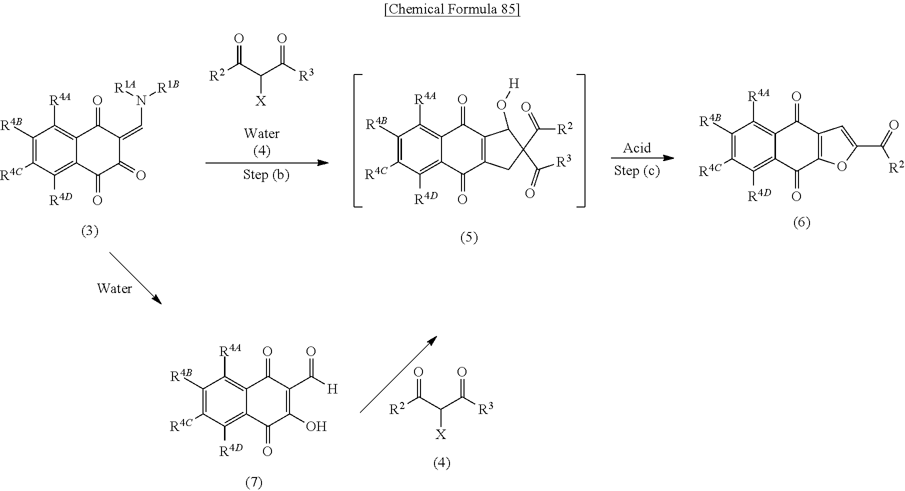

wherein R^{1A}, R^{1B}, R², R³, R^{4A}, R^{4B}, R^{4C}, and R^{4D} are defined the same as item 1 and/or other items.

Regarding Step (b)

This is a step for converting a compound represented by formula (3) obtained using formula (1) and formula (2a) or formula (2b) in manufacturing step (a) described above into a compound represented by formula (5) by adding water in the presence of a compound represented by formula (4) in a solvent. Step (b) is characterized by conversion into a compound represented by formula (5) via 3-hydroxy-1,4-dioxo-1,4-dihydronaphthalene-2-carbaldehyde (7) of a salt thereof by hydrolysis with water.

Examples of solvents used in this step include water, methanol, ethanol, acetone, diethyl ether, dichloromethane, chloroform, tetrahydrofuran, acetonitrile, dimethyl sulfoxide, N,N-dimethylacetamide, N,N-dimethylformamide, N-methyl-2-pyrrolidone, and a mixture solvent thereof. The solvent is preferably dimethyl sulfoxide, N,N-dimethylacetamide, N,N-dimethylformamide, or N-methyl-2-pyrrolidone, and more preferably N-methyl-2-pyrrolidone.

Another preferred embodiment as a solvent used in this step includes an amide-based solvent.

The amount of solvent used in this step is generally 4-fold to 10-fold in weight, preferably 5-fold to 10-fold in weight, and more preferably 6-fold to 9-fold in weight with respect to 1 weight of a compound represented by formula (3).

The amount of compound represented by formula (4) used is generally 1.0 equivalent to 10.0 equivalent, preferably 1.0 equivalent to 5.0 equivalent, more preferably 1.0 equivalent to 2.0 equivalent, and most preferably 1.0 equivalent to 1.5 equivalent with respect to 1 equivalent of a compound represented by formula (3).

The amount of water used is generally 0.0 equivalent to 50.0 equivalent, preferably 1.0 equivalent to 40.0 equivalent, more preferably 2.0 equivalent to 20.0 equivalent, and most preferably 5.0 equivalent to 10.0 equivalent with respect to 1 equivalent of a compound represented by formula (3).

The reaction time is generally about 4 hours to 20 hours, preferably about 5 hours to 15 hours, and more preferably about 6 hours to 10 hours.

The reaction temperature is generally 0° C. to 100° C., preferably 40° C. to 80° C., and more preferably 40° C. to 60° C.

A quaternary ammonium salt can be added as an additive for promoting a reaction. The amount used is preferably 0.5 equivalent to 5.0 equivalent. Examples of quaternary ammonium salts include tetrabutylammonium fluoride, tetrabutylammonium chloride, tetrabutylammonium bromide, tetrabutylammonium iodide, tetrabutylammonium hydroxide, tetrabutylammonium hydrogen sulfate, and the like. A quaternary ammonium salt is preferably tetrabutylammonium fluoride, tetrabutylammonium chloride, or tetrabutylammonium bromide, and more preferably tetrabutylammonium chloride or tetrabutylammonium bromide.

Regarding Step (c)

Step (c) is a step for obtaining 2-alkylcarbonylnaphtho[2,3-b]furan-4,9-dione set forth in formula (6) via an elimination reaction by heating the compound represented by formula (5) obtained in manufacturing step (b) described above.

Examples of solvents used in step (c) include water, methanol, ethanol, acetone, diethyl ether, dichloromethane, chloroform, tetrahydrofuran, acetonitrile, dimethyl sulfoxide, N,N-dimethylacetamide, N,N-dimethylformamide, N-methyl-2-pyrrolidone, and a mixture solvent thereof. The solvent is preferably dimethyl sulfoxide, N,N-dimethylacetamide, N,N-dimethylformamide, or N-methyl-2-pyrrolidone, and more preferably N-methyl-2-pyrrolidone.

The amount of solvent used in step (c) is generally 4-fold to 10-fold in weight, preferably 5-fold to 10-fold in weight, and more preferably 6-fold to 9-fold in weight with respect to 1 weight of a compound represented by formula (5).

Examples of acids used in step (c) include hydrochloric acid, hydrobromic acid, trifluoroacetic acid, sulfuric acid, methanesulfonic acid, p-toluenesulfonic acid, formic acid, and acetic acid. The acid is preferably hydrochloric acid or sulfuric acid.

The amount of acid used (amount added) in this step is generally 0.1 equivalent to 10.0 equivalent, preferably 0.5 equivalent to 5.0 equivalent, more preferably 1.0 equivalent to 4.0 equivalent, still more preferably 1.5 equivalent to 3.5 equivalent, and most preferably 2.0 equivalent to 3.0 equivalent with respect to 1 equivalent of a compound represented by formula (3).

The reaction time is generally about 3 hours to 20 hours, preferably about 4 hours to 15 hours, and more preferably about 5 hours to 10 hours.

The reaction temperature is generally 20° C. to 150° C., preferably 40° C. to 140° C., more preferably 50° C. to 130° C., still more preferably 60° C. to 120° C., and most preferably 60° C. to 100° C.

When performing step (b) and step (c) in one-pot reaction

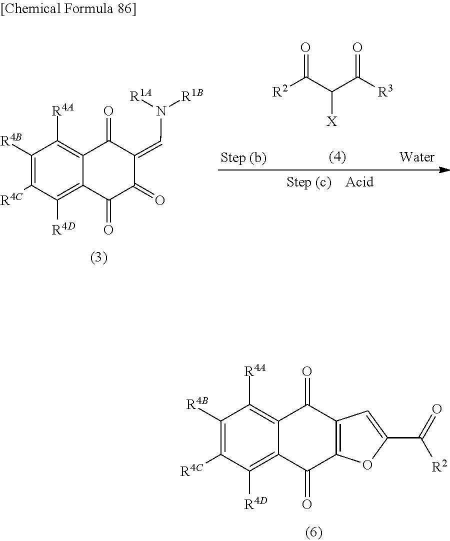

Step (b) and step (c) can be performed by first isolating the compound represented by formula (5) obtained in step (b), as well as by performing step (b) and step (c) consecutively in one-pot without isolating the compound represented by formula (5).

When performing step (b) and step (c) in one-pot, a solvent is preferably N,N-dimethylformamide or N-methyl-2-pyrrolidone, and more preferably N-methyl-2-pyrrolidone.

When performing step (b) and step (c) in one-pot, the amount of solvent used is generally 4-fold to 10-fold in weight, preferably 5-fold to 10-fold in weight, and more preferably 6-fold to 9-fold in weight with respect to 1 weight of a compound represented by formula (3).

When performing step (b) and step (c) in one-pot, the amount of compound represented by formula (4) used is generally 1.0 equivalent to 10.0 equivalent, preferably 1.0 equivalent to 5.0 equivalent, more preferably 1.0 equivalent to 2.0 equivalent, and most preferably 1.0 equivalent to 1.5 equivalent with respect to 1 equivalent of a compound represented by formula (3).

When performing step (b) and step (c) in one-pot, the amount of water used is generally 0.0 equivalent to 50.0 equivalent, preferably 1.0 equivalent to 40.0 equivalent, more preferably 2.0 equivalent to 20.0 equivalent, and most preferably 5.0 equivalent to 10.0 equivalent with respect to 1 equivalent of a compound represented by formula (3).

When performing step (b) and step (c) in one-pot, the reaction time in step (b) is generally about 4 hours to 20 hours, preferably about 5 hours to 15 hours, and more preferably about 6 hours to 10 hours.

When performing step (b) and step (c) in one-pot, the reaction time in step (c) is generally about 3 hours to 20 hours, preferably about 4 hours to 15 hours, and more preferably about 5 hours to 10 hours.

When performing step (b) and step (c) in one-pot, the reaction temperature in step (b) is generally 0° C. to 100° C., preferably 40° C. to 80° C., and more preferably 40° C. to 60° C.

When performing step (b) and step (c) in one-pot, the reaction temperature in step (c) is generally 20° C. to 150° C., preferably 40° C. to 140° C., more preferably 50° C. to 130° C., still more preferably 60° C. to 120° C., and most preferably 60° C. to 100° C.

When performing step (b) and step (c) in one-pot, a quaternary ammonium salt can be added as an additive for promoting a reaction in step (b). The amount used is preferably 0.5 equivalent to 5.0 equivalent. Examples of quaternary ammonium salts include tetrabutylammonium fluoride, tetrabutylammonium chloride, tetrabutylammonium bromide, tetrabutylammonium iodide, tetrabutylammonium hydroxide, tetrabutylammonium hydrogen sulfate, and the like. A quaternary ammonium salt is preferably tetrabutylammonium fluoride, tetrabutylammonium chloride, or tetrabutylammonium bromide, and more preferably tetrabutylammonium chloride or tetrabutylammonium bromide.

When performing step (b) and step (c) in one-pot, an acid used in step (c) is hydrochloric acid, hydrobromic acid, trifluoroacetic acid, sulfuric acid, methanesulfonic acid, p-toluenesulfonic acid, formic acid, or acetic acid, and is preferably hydrochloric acid or sulfuric acid.

The amount of acid used (amount added) in this step is generally 0.1 equivalent to 10.0 equivalent, preferably 0.5 equivalent to 5.0 equivalent, more preferably 1.0 equivalent to 4.0 equivalent, still more preferably 1.5 equivalent to 3.5 equivalent, and most preferably 2.0 equivalent to 3.0 equivalent with respect to 1 equivalent of a compound represented by formula (3).

Furthermore, steps (a), (b), and (c) can be performed consecutively in one-pot without isolating the compounds represented by formulas (3) and (5). Specifically, a compound represented by formula (6) can be directly obtained from a compound represented by formula (1).

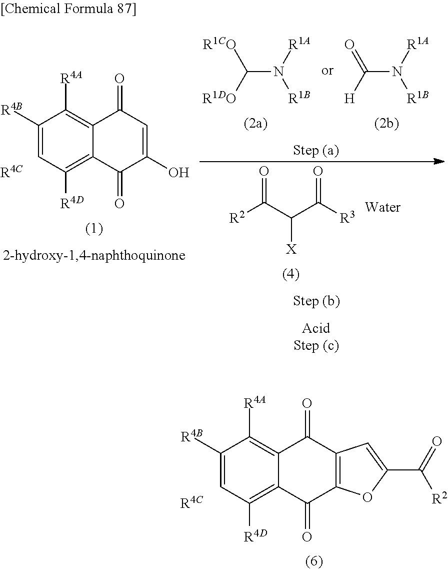

When performing steps (a), (b), and (c) in one-pot, the solvent is water, methanol, ethanol, acetone, diethyl ether, dichloromethane, chloroform, tetrahydrofuran, acetonitrile, dimethyl sulfoxide, N,N-dimethylacetamide, N,N-dimethylformamide, N-methyl-2-pyrrolidone, pyridine, or a mixture solvent thereof, and preferably N,N-dimethylformamide or N-methyl-2-pyrrolidone.

When performing steps (a), (b), and (c) in one-pot, the amount of solvent used is generally 3-fold to 10-fold in weight, preferably 4-fold to 9-fold in weight, and more preferably 5-fold to 9-fold in weight with respect to 1 weight of 2-hydroxy-1,4-naphthoquinone.

When performing steps (a), (b), and (c) in one-pot, the amount of compound represented by formula (2a) or formula (2b) used in step (a) is generally 1.0 equivalent to 10.0 equivalent, preferably 1.0 equivalent to 5.0 equivalent, more preferably 1.0 equivalent to 2.0 equivalent, and most preferably 1.0 equivalent to 1.5 equivalent with respect to 1 equivalent of 2-hydroxy-1,4-naphthoquinone.

When performing steps (a), (b), and (c) in one-pot, the amount of compound represented by formula (4) used in step (b) is generally 1.0 equivalent to 10.0 equivalent, preferably 1.0 equivalent to 5.0 equivalent, more preferably 1.0 equivalent to 2.0 equivalent, and most preferably 1.0 equivalent to 1.5 equivalent with respect to 1 equivalent of 2-hydroxy-1,4-naphthoquinone.

When performing steps (a), (b), and (c) in one-pot, the amount of water used in step (b) is generally 0.0 equivalent to 50.0 equivalent, preferably 1.0 equivalent to 40.0 equivalent, more preferably 2.0 equivalent to 20.0 equivalent, and most preferably 5.0 equivalent to 10.0 equivalent with respect to 1 equivalent of 2-hydroxy-1,4-naphthoquinone.

When performing steps (a), (b), and (c) in one-pot, an acid used in step (c) is hydrochloric acid, hydrobromic acid, trifluoroacetic acid, sulfuric acid, methanesulfonic acid, p-toluenesulfonic acid, formic acid, or acetic acid, and is preferably hydrochloric acid or sulfuric acid.

When performing steps (a), (b), and (c) in one-pot, the amount of acid used in the one-pot step is generally 0.1 equivalent to 10.0 equivalent, preferably 0.5 equivalent to 5.0 equivalent, more preferably 1.0 equivalent to 4.0 equivalent, still more preferably 1.5 equivalent to 3.5 equivalent, and most preferably 2.0 equivalent to 3.0 equivalent with respect to 1 equivalent of 2-hydroxy-1,4-naphthoquinone.

When performing steps (a), (b), and (c) in one-pot, the reaction time in step (a) is generally about 0.5 hours to 12 hours, and preferably about 0.5 hours to 8 hours.

When performing steps (a), (b), and (c) in one-pot, the reaction time in step (b) is generally about 4 hours to 20 hours, preferably about 5 hours to 15 hours, and more preferably about 5 hours to 10 hours.

When performing steps (a), (b), and (c) in one-pot, the reaction time in step (c) is generally about 3 hours to 20 hours, preferably about 4 hours to 15 hours, and more preferably about 5 hours to 13 hours.

When performing steps (a), (b), and (c) in one-pot, the reaction temperature in step (a) is generally −30° C. to 120° C., preferably −10° C. to 130° C., more preferably −10° C. to 10° C., and still more preferably −5° C. to 5° C.

When performing steps (a), (b), and (c) in one-pot, the reaction temperature in step (b) is generally 0° C. to 100° C., preferably 40° C. to 80° C., and more preferably 40° C. to 60° C.

When performing steps (a), (b), and (c) in one-pot, the reaction temperature in step (c) is generally 20° C. to 150° C., preferably 40° C. to 140° C., more preferably 50° C. to 130° C., still more preferably 60° C. to 120° C., and most preferably 60° C. to 100° C.

When performing steps (a), (b), and (c) in one-pot, a quaternary ammonium salt can be added as an additive for promoting a reaction in step (b). The amount used is preferably 0.5 equivalent to 5.0 equivalent. Examples of quaternary ammonium salts include tetrabutylammonium fluoride, tetrabutylammonium chloride, tetrabutylammonium bromide, tetrabutylammonium iodide, tetrabutylammonium hydroxide, tetrabutylammonium hydrogen sulfate, and the like. A quaternary ammonium salt is preferably tetrabutylammonium fluoride, tetrabutylammonium chloride, or tetrabutylammonium bromide, and more preferably tetrabutylammonium chloride or tetrabutylammonium bromide.

A naphtho[2,3-b]furan-4,9-dione backbone with a substitution at position 2 can be manufactured cost-effectively and safely at a high purity and high yield by the manufacturing methods (a), (b), and (c) of the present disclosure. Therefore, a related substance that is useful as an intermediate of 2-alkylcarbonylnaphtho[2,3-b]furan-4,9-dione can be manufactured more cost effectively and more safely at a higher purity and higher yield compared to conventional methods. Furthermore, 2-alkylcarbonylnaphtho[2,3-b]furan-4,9-dione, which is useful as a pharmaceutical product, can be manufactured cost effectively, safely, and conveniently by performing step (c) on said intermediate.

The compound of formula (1) is a commercially available, readily obtainable, cost effective, safe, and stable compound. By using such a compound, a related substance (compound of formula (3)) of 2-alkylcarbonylnaphtho[2,3-b]furan-4,9-dione can be manufactured cost effectively, safely, and conveniently at a high yield and high purity by performing step (a) in N,N-dimethylformamide using only the compound represented by formula (2a) or formula (2b). Surprisingly, heating, quenching, concentration, and separation steps are not required whatsoever under the reaction conditions described above. The intermediate (compound of formula (3)) can be manufactured through only filtration.

With the compound of formula (3) obtained in step (a), a related substance (compound of formula (5)) of 2-alkylcarbonylnaphtho[2,3-b]furan-4,9-dione can be manufactured safely, conveniently, and cost effectively at a high yield and high purity through step (b). Surprisingly, formula (5) does not require isolation under the reaction condition described above. 2-alkylcarbonylnaphtho[2,3-b]furan-4,9-dione can be manufactured by subsequently performing step (c). Furthermore, said step also requires no quenching, concentration, or separation step. 2-alkylcarbonylnaphtho[2,3-b]furan-4,9-dione can be manufactured through only filtration. Said step is extremely useful in the manufacture of 2-alkylcarbonylnaphtho[2,3-b]furan-4,9-dione and related substances thereof from the viewpoint of environmental impact and industrial productivity.

Manufacturing Method 2

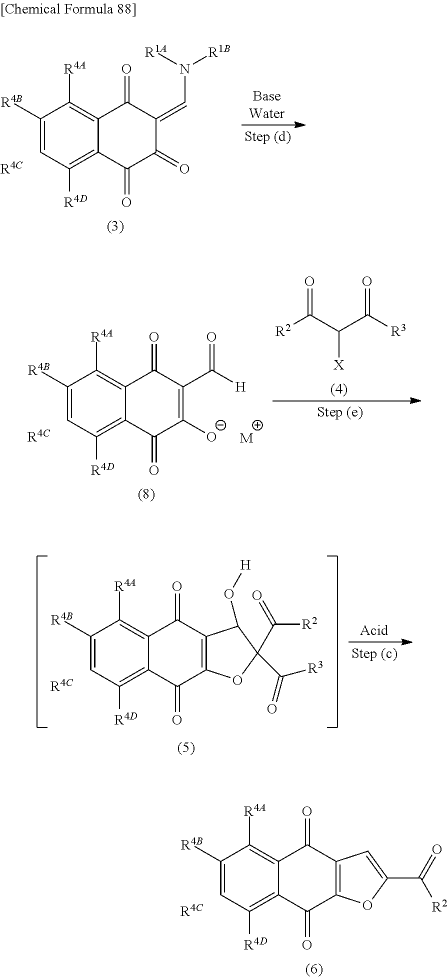

wherein M, R^{1A}, R^{1B}, R², R³, R^{4A}, R^{4B}, R^{4C}, and R^{4D} are defined the same as item 1 and/or other items.

The method of the present disclosure associated with Manufacturing Method 2 obtains aldehyde (8) by treating the compound represented by formula (3) obtained by Manufacturing Method 1 described above with a base at step (d). This is a method, which subsequently obtains a compound represented by formula (5) by reacting a compound represented by formula (8) with a compound represented by formula (4) in step (e), and then obtains the compound represented by formula (6) by the same method as step (c) in Manufacturing Method 1 described above. Step (e) is a step of constructing a 2-alkylcarbonyl-3-hydroxy-2,3-dihydronaphtho[2,3-b]furan-4,9-dione backbone, which is one of the most important feature in Manufacturing Method 2. Steps (d) and (e) are described hereinafter with a preferred embodiment, but the present disclosure is not limited thereto.

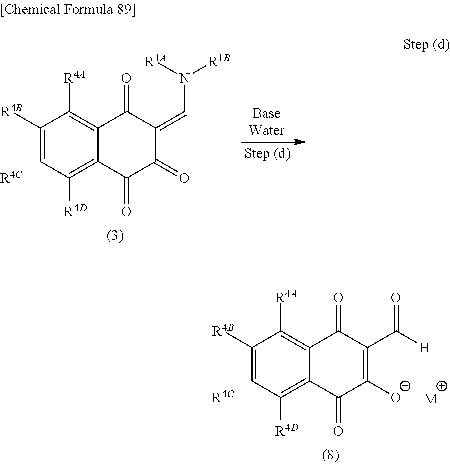

wherein M, R^{1A}, R^{1B}, R^{1C}, R^{1D}, R^{4A}, R^{4B}, R^{4A}, and R^{4B} are defined the same as item 1 and/or other items.

This is a step for obtaining a compound represented by formula (8) by reacting the compound represented by formula (3) obtained in step (a) with a base and water.

The solvent used in this step is not particularly limited, as long as the boiling point is at or above the reaction temperature of this reaction. Examples thereof include ethylene glycol, N,N-dimethylacetamide, N,N-dimethylformamide, dimethyl sulfoxide, N-methyl-2-pyrrolidone, monochlorobenzene, and toluene. The solvent is preferably N,N-dimethylacetamide, N,N-dimethylformamide, N-methyl-2-pyrrolidone, or toluene, more preferably N,N-dimethylformamide or N-methyl-2-pyrrolidone, and still more preferably N,N-dimethylformamide.

Another preferred embodiment as a solvent used in this step includes an amide-based solvent.

The amount of solvent used in this step is generally 3-fold to 30-fold in weight, preferably 5-fold to 30-fold in weight, and more preferably 10-fold to 30-fold in weight with respect to 1 weight of a compound represented by formula (3).

Examples of bases used in this step include inorganic bases such as ammonium, lithium hydroxide, sodium hydroxide, calcium hydroxide, barium hydroxide, sodium carbonate, sodium hydrogen carbonate, potassium carbonate, and cesium carbonate, and organic bases such as pyridine, N,N-diisopropylethylamine, and triethylamine. The base is preferably lithium hydroxide, sodium hydroxide, sodium carbonate, potassium carbonate, sodium hydrogen carbonate, or cesium carbonate, more preferably sodium carbonate, potassium carbonate, sodium hydrogen carbonate, or cesium carbonate, and most preferably potassium carbonate.

The amount of based used in this step is generally 1.0 equivalent to 10.0 equivalent, preferably 1.0 equivalent to 5.0 equivalent, more preferably 1.0 equivalent to 2.0 equivalent, and most preferably 1.0 equivalent to 1.5 equivalent with respect to 1 equivalent of a compound represented by formula (3).

The reaction time is generally about 0.5 hours to 5 hours, and preferably 1 hour to 3 hours.

The reaction temperature is generally −30° C. to 100° C., preferably −10° C. to 50° C., and more preferably 0° C. to 30° C.

The amount of water used in this step is generally 0.1-fold to 10-fold in weight, preferably 0.5-fold to 5-fold in weight, and more preferably 0.8-fold to 4-fold in weight with respect to 1 weight of a compound represented by formula (3).

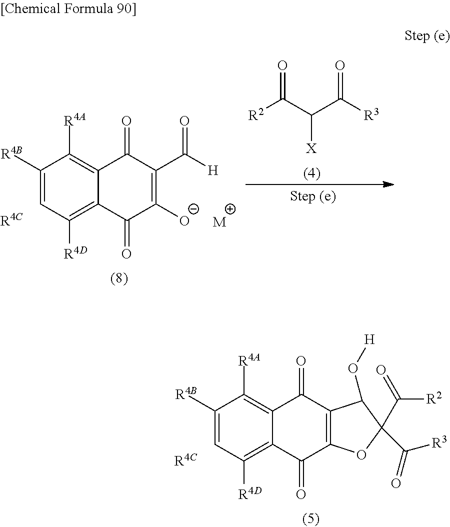

wherein M, R², R, R^{4A}, R^{4B}, R^{4A}, and R^{4B} are defined the same as item 1 and/or other items.

This is a step for obtaining a compound represented by formula (5) by reacting the compound represented by formula (8) obtained in step (d) with a compound represented by formula (4).

The solvent used in this step is not particularly limited, as long as the boiling point is at or above the reaction temperature of this reaction. Examples thereof include ethylene glycol, N,N-dimethylacetamide, N,N-dimethylformamide, dimethyl sulfoxide, N-methyl-2-pyrrolidone, monochlorobenzene, and toluene. The solvent is preferably N,N-dimethylacetamide, N,N-dimethylformamide, N-methyl-2-pyrrolidone, or toluene, more preferably N,N-dimethylformamide or N-methyl-2-pyrrolidone, and still more preferably N-methyl-2-pyrrolidone.

Another preferred embodiment as a solvent used in this step includes an amide-based solvent.

The amount of solvent used in this step is generally 4-fold to 10-fold in weight, preferably 5-fold to 10-fold in weight, and more preferably 6-fold to 9-fold in weight with respect to 1 weight of a compound represented by formula (3).

The amount of compound represented by formula (4) used is generally 1.0 equivalent to 10.0 equivalent, preferably 1.0 equivalent to 5.0 equivalent, more preferably 1.0 equivalent to 2.0 equivalent, and most preferably 1.0 equivalent to 1.5 equivalent with respect to 1 equivalent of the compound represented by formula (8).

The reaction time is generally about 4 hours to 20 hours, preferably about 5 hour to 18 hours, and more preferably about 6 hours to 15 hours.

The reaction temperature is generally 0° C. to 100° C., preferably 40° C. to 80° C., and more preferably 40° C. to 60° C.

Step (e) and step (c) can be performed by first isolating the compound represented by formula (5) obtained in step (e), as well as by performing step (e) and step (c) consecutively in one-pot without isolating the compound represented by formula (5).

When performing step (e) and step (c) in one-pot, a solvent is preferably N,N-dimethylformamide or N-methyl-2-pyrrolidone, and more preferably N-methyl-2-pyrrolidone.

When performing step (e) and step (c) in one-pot, the amount of solvent used is generally 4-fold to 10-fold in weight, preferably 5-fold to 10-fold in weight, and more preferably 6-fold to 9-fold in weight with respect to 1 weight of a compound represented by formula (8).

When performing step (e) and step (c) in one-pot, the amount of compound represented by formula (4) used is generally 1.0 equivalent to 10.0 equivalent, preferably 1.0 equivalent to 5.0 equivalent, more preferably 1.0 equivalent to 2.0 equivalent, and most preferably 1.0 equivalent to 1.5 equivalent with respect to 1 equivalent of a compound represented by formula (8).

When performing step (e) and step (c) in one-pot, the reaction time in step (e) is generally about 4 hours to 20 hours, preferably about 5 hours to 15 hours, and more preferably about 6 hours to 10 hours.

When performing step (e) and step (c) in one-pot, the reaction time in step (c) is generally about 3 hours to 30 hours, preferably about 4 hours to 25 hours, and more preferably about 5 hours to 20 hours.

When performing step (e) and step (c) in one-pot, the reaction temperature in step (e) is generally 0° C. to 100° C., preferably 40° C. to 80° C., and more preferably 40° C. to 70° C.

When performing step (e) and step (c) in one-pot, the reaction temperature in step (c) is generally 20° C. to 150° C., preferably 40° C. to 140° C., more preferably 50° C. to 130° C., still more preferably 60° C. to 120° C., and most preferably 60° C. to 100° C.

When performing step (e) and step (c) in one-pot, an acid used in step (c) is hydrochloric acid, hydrobromic acid, trifluoroacetic acid, sulfuric acid, methanesulfonic acid, p-toluenesulfonic acid, formic acid, or acetic acid, and is preferably hydrochloric acid or sulfuric acid.

The amount of acid used (amount added) in this step is generally 0.1 equivalent to 10.0 equivalent, preferably 0.5 equivalent to 5.0 equivalent, more preferably 1.0 equivalent to 4.0 equivalent, still more preferably 1.5 equivalent to 3.5 equivalent, and most preferably 2.0 equivalent to 3.0 equivalent with respect to 1 equivalent of a compound represented by formula (8).

Furthermore, steps (a) and (d) can be performed consecutively in one-pot without isolating the compound represented by formula (3). Specifically, a compound represented by formula (8) can be directly obtained from a compound represented by formula (1).

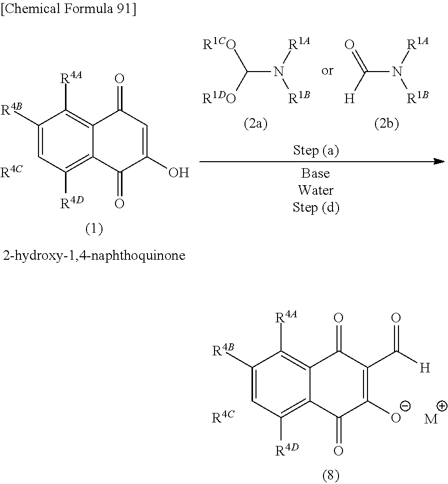

When performing step (a) and step (d) in one-pot, the solvent is preferably N,N-dimethylformamide or N-methyl-2-pyrrolidone, and more preferably N,N-dimethylformamide.

When performing step (a) and step (d) in one-pot, the amount of solvent used is generally 4-fold to 10-fold in weight, preferably 5-fold to 10-fold in weight, and more preferably 6-fold to 9-fold in weight with respect to 1 weight of 2-hydroxy-1,4-naphthoquinone.

When performing step (a) and step (d) in one-pot, the amount of compound represented by formula (2a) or formula (2b) used in step (a) is generally 1.0 equivalent to 10.0 equivalent, preferably 1.0 equivalent to 5.0 equivalent, more preferably 1.0 equivalent to 2.0 equivalent, and most preferably 1.0 equivalent to 1.5 equivalent with respect to 1 equivalent of 2-hydroxy-1,4-naphthoquinone.

When performing step (a) and step (d) in one-pot, the base used in step (d) is an inorganic base such as ammonium, lithium hydroxide, sodium hydroxide, calcium hydroxide, barium hydroxide, sodium carbonate, sodium hydrogen carbonate, potassium carbonate, or cesium carbonate, or an organic base such as pyridine, N,N-diisopropylethylamine, or triethylamine. The base is preferably lithium hydroxide, sodium hydroxide, sodium carbonate, potassium carbonate, sodium hydrogen carbonate, or cesium carbonate, more preferably sodium carbonate, potassium carbonate, sodium hydrogen carbonate, or cesium carbonate, and most preferably potassium carbonate.

When performing step (a) and step (d) in one-pot, the amount of based used in step (d) is generally 1.0 equivalent to 10.0 equivalent, preferably 1.0 equivalent to 5.0 equivalent, more preferably 1.0 equivalent to 2.0 equivalent, and most preferably 1.0 equivalent to 1.5 equivalent with respect to 1 equivalent of 2-hydroxy-1,4-naphthoquinone.

When performing step (a) and step (d) in one-pot, the amount of water used in step (d) is generally 0.0 equivalent to 50.0 equivalent, preferably 1.0 equivalent to 40.0 equivalent, more preferably 2.0 equivalent to 20.0 equivalent, and most preferably 5.0 equivalent to 10.0 equivalent with respect to 1 equivalent of 2-hydroxy-1,4-naphthoquinone.

When performing step (a) and step (d) in one-pot, the reaction time in step (a) is generally about 0.5 hours to 12 hours, and preferably about 0.5 hours to 8 hours.

When performing step (a) and step (d) in one-pot, the reaction time in step (d) is generally about 0.5 hours to 5 hours, and preferably about 1 hour to 3 hours.

When performing step (a) and step (d) in one-pot, the reaction temperature in step (a) is generally −30° C. to 120° C., preferably −10° C. to 130° C., more preferably −10° C. to 10° C., and still more preferably −5° C. to 5° C.

When performing step (a) and step (d) in one-pot, the reaction temperature in step (d) is generally −30° C. to 100° C., preferably −10° C. to 50° C., and more preferably 0° C. to 30° C.

The order of adding reagents or the like is not limited to the order described above.

The present disclosure is described in further detail hereinafter with Reference Examples and Examples, but the present disclosure is not limited thereby. Compounds were identified with an elemental analysis value, a mass spectrum, a high performance liquid chromatography mass spectrometer (LCMS), infrared (IR) absorption spectra, nuclear magnetic resonance (NMR) spectra, high performance liquid chromatography (HPLC), or the like.

The measurement conditions for high performance liquid chromatography (HPLC) are described below. The retention time is indicated by Rt (minutes). The measurement conditions used for measurement are described in each of the actual measurement values. In the following description, HPLC purity (area %) calculates purity by comparing each peak area using the following measurement conditions.

### Column: Kinetex 1.7u C18 100 Å (100×2.1 mm)

Eluent: solution A: aqueous 0.05% trifluoroacetic acid solution, solution B: 0.05% trifluoroacetic acid-acetonitrile solution

### Gradient Condition:

TABLE 1

| Minutes | A (%) | B (%) |
|---|---|---|
| 0-1 | 95 | 5 |
| 1-9 | 95→10 | 5→90 |
| 9-13 | 10 | 90 |
| 13-13.1 | 10→95 | 90→5 |
| 13.1-18 | 95 | 5 |

Flow rate: 0.3 mL/min
Column temperature: 40° C.

### Wavelength: 254 nm

Rt for each compound measured under the above measurement conditions is shown in the following Table.

TABLE 2

| Compound | Rt (minutes) |
|---|---|
| 2-hydroxy-1,4-naphthoquinone (1) | 5.7 |
| (3E)-3- | 5.1 |
| [(dimethylamino)methylidene]naphthalene- | |
| 1,2,4(3H)-trione (3) | |
| 3-hydroxy-1,4-dioxo-1,4- | 4.5 |
| dihydronaphthalene-2-carbaldehyde (7) | |
| 2,2-diacetyl-3-hydroxy-2,3- | 6.3 |
| dihydronaphtho[2,3-b]furan-4,9-dione (5) | |
| 2-acetylnaphtho[2,3-b]furan-4,9-dione (6) | 7.1 |

The following abbreviations may be used in the Reference Examples, Examples, and Tables in the Examples to simplify the descriptions herein.
Me: methyl
DMF: N,N-dimethylformamide
NMP: N-methyl-2-pyrrolidone
TFA: trifluoroacetic acid
THF: tetrahydrofuran
DMSO: dimethyl sulfoxide
wt %: weight %
   As symbols used in NMR, s refers to singlet, d refers to doublet, t refers to triplet, q refers to quartet, and m refers to multiplet.
   Room temperature is 10° C. to 30° C.

### EXAMPLES

While the present disclosure is described in more detail hereinafter with Examples and Reference Examples, the technical scope of the present disclosure is not limited to such Examples. The present disclosure may be altered to the extent that the altered invention remains within the scope of the present disclosure. It should be noted that compound names in the following Examples and Comparative Examples do not necessarily follow the IUPAC nomenclature.

### Example 1: Manufacturing Method of 2-acetylnaphtho[2,3-b]furan-4,9-dione

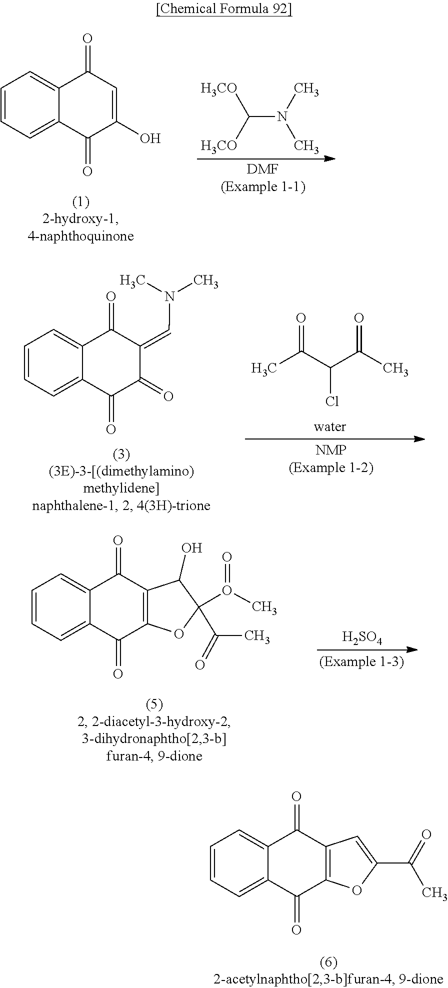

### Example 1-1: Manufacturing Method of (3E)-3-[(dimethylamino)methylidene]naphthalene-1,2,4(3H)-trione

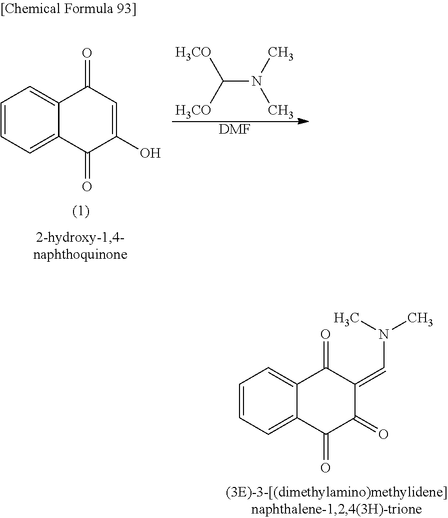

Under a nitrogen atmosphere, a DMF (200.4 kg) solution of 2-hydroxy-1,4-naphthoquinone (50.00 kg) was added dropwise over 2 hours at 0±5C to a DMF (150.2 kg) solution of N,N-dimethylformamide dimethyl acetal (51.4 kg). (3E)-3-[(dimethylamino)methylidene]naphthalene-1,2,4(3H)-trione started to elute out during the addition. A crystal eluted after stirring for 5 hours at 0±5° C. was filtered, then washed with methanol, and dried under reduced pressure to obtain (3E)-3-[(dimethylamino)methylidene]naphthalene-1,2,4(3H)-trione (46.28 kg, yield: 70%, HPLC purity: 99.89 area %).

¹H-NMR (400 MHz, DMSO-d₆) δ: 8.51 (s, 1H), 8.09 (dd, 1H, J=7.3, 1.2 Hz), 7.98 (dd, 1H, J=7.3, 1.2 Hz), 7.85 (ddd, 1H, J=7.9, 7.3, 1.2 Hz), 7.76 (ddd, 1H, J=7.9, 7.3, 1.2 Hz), 3.61 (s, 3H), 3.23 (s, 3H).

### Example 1-2: Manufacturing Method of 2,2-diacetyl-3-hydroxy-2,3-dihydronaphtho[2,3-b]furan-4,9-dione

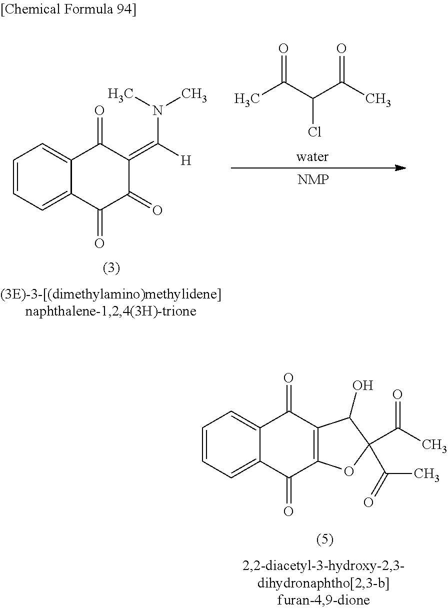

Under a nitrogen atmosphere, 3-chloropentane-2,4-dione (11.74 g) and water (1.97 g) were added at room temperature to an NMP (40 g) solution of (3E)-3-[(dimethylamino)methylidene]naphthalene-1,2,4(3H)-trione (5.00 g) obtained in Example 1-1 described above. The temperature was raised to 50° C. The mixture was stirred for 7 hours at 50° C. After cooling the mixture to room temperature, water (100 mL) and ethyl acetate (100 mL) were added, and the organic layer was separated. The organic layer was washed with water (100 mL) and saturated saline (50 mL), dried with sodium sulfate, and then concentration under reduced pressure. After concentration, the eluted solid was filtered, washed four times with chloroform (5 mL), dried under reduced pressure at 50° C. to obtain the compound of interest (4.47 g, yield: 68%, HPLC purity: 88.47 area %, 2,2-diacetyl-3-hydroxy-2,3-dihydronaphtho[2,3-b]furan-4,9-dione isomer (2,2-diacetyl-3-hydroxy-2,3-dihydronaphtho[1,2-b]furan-4,5-dione): 11.47 area %, 2-acetylnaphtho[2,3-b]furan-4,9-dione: 0.06 area %).

¹H-NMR (400 MHz, CDCl₃) δ: 8.21-8.06 (m, 2H), 7.85-7.70 (m, 2H), 6.11 (d, 1H, J=4.9 Hz), 3.15 (d, 1H, J=4.9 Hz), 2.43 (s, 3H), 2.32 (s, 3H).

### Example 1-3: Manufacturing Method of 2-acetylnaphtho[2,3-b]furan-4,9-dione

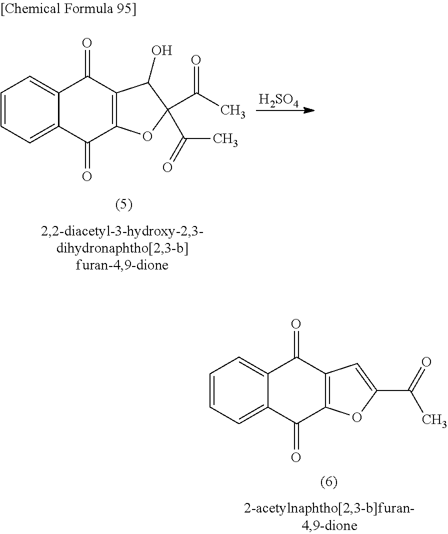

Concentrated sulfuric acid (0.92 g) was added to an NMP (8.4 mL) solution of the 2,2-diacetyl-3-hydroxy-2,3-dihydronaphtho[2,3-b]furan-4,9-dione (1.08 g) obtained in Example 1-2 described above, and the mixture was stirred for 8 hours at 90° C. The reaction solution was cooled, and then additional concentrated sulfuric acid (0.92 g) was added. The mixture was stirred for 17 hours at 90° C. After cooling to 20° C., methanol (3.3 g) was added. The mixture was stirred for 3 hours at 20° C., and then the eluted solid was filtered out. The solid was washed with methanol, then dried under reduced pressure at 50° C. to obtain the compound of interest (0.61 g, yield: 71%, HPLC purity: 99.77 area %)

¹H-NMR (400 MHz, CDCl₃) δ: 8.31-8.20 (m, 2H), 7.85-7.78 (m, 2H), 7.61 (s, 1H), 2.67 (s, 3H).

Aggregate yield based on 2-hydroxy-1,4-naphthoquinone: 34%

### Example 2: Manufacturing Method of 2-acetylnaphtho[2,3-b]furan-4,9-dione

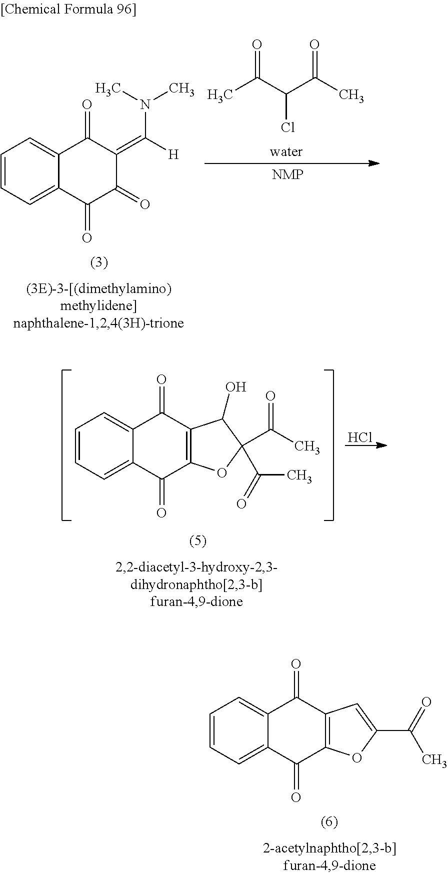

Under a nitrogen atmosphere, 3-chloropentane-2,4-dione (32.87 kg) and water (31.43 kg) were added to an NMP (188.1 kg) solution of the (3E)-3-[(dimethylamino)methylidene]naphthalene-1,2,4(3H)-trione (40.0 kg) obtained in Example 1-1 described above. The temperature was raised to 50±10° C. The mixture was stirred for 6 hours (2,2-diacetyl-3-hydroxy-2,3-dihydronaphtho[2,3-b]furan-4,9-dione solution). In another reaction vessel, a mixture of water (80.2 kg) and concentrated hydrochloric acid (90.88 kg) and NMP (40.00 kg) was prepared at room temperature, and the temperature was raised to 70±10° C. A 2,2-diacetyl-3-hydroxy-2,3-dihydronaphtho[2,3-b]furan-4,9-dione solution prepared in advance was maintained at a temperature of 50±10° C. and added dropwise to the hydrochloric acid water-NMP mixture at 70±10° C. After stirring for 7 hours at 70±10° C., methanol (160 kg) was added at 65° C. or lower. The mixture was cooled to 20±5° C. then stirred for 12 hours at 20±5° C. The eluted solid was filtered, washed four times with methanol (120 kg), and then dried by aeration with nitrogen at 50° C. or lower to obtain a crude crystal of the compound of interest (33.67 kg, yield: 80%, HPLC purity: 99.23 area %).

Under a nitrogen atmosphere, activated carbon (1.50 kg) and anisol (570.0 kg) were added to the resulting crude crystal (30.00 kg) at room temperature. The temperature was raised to 100±10° C., and the mixture was stirred for 2 hours. Activated carbon was removed by filtration at 100±10° C., and the filtrate was washed with anisol (30.00 kg). The resulting filtrate was cooled to −5±5° C. at a cooling rate of 10° C./hour, and stirred for 1 hour or longer at −5±5° C. The eluted solid was filtered. The solid was washed twice with ethyl acetate (120.0 kg) and then dried by aeration with nitrogen at 60° C. or lower to obtain the compound of interest (27.22 kg, yield: 91%, HPLC purity: 99.98 area %).

Aggregate yield based on 2-hydroxy-1,4-naphthoquinone: 51%

### Example 3: Manufacturing Method of 2-acetylnaphtho[2,3-b]furan-4,9-dione

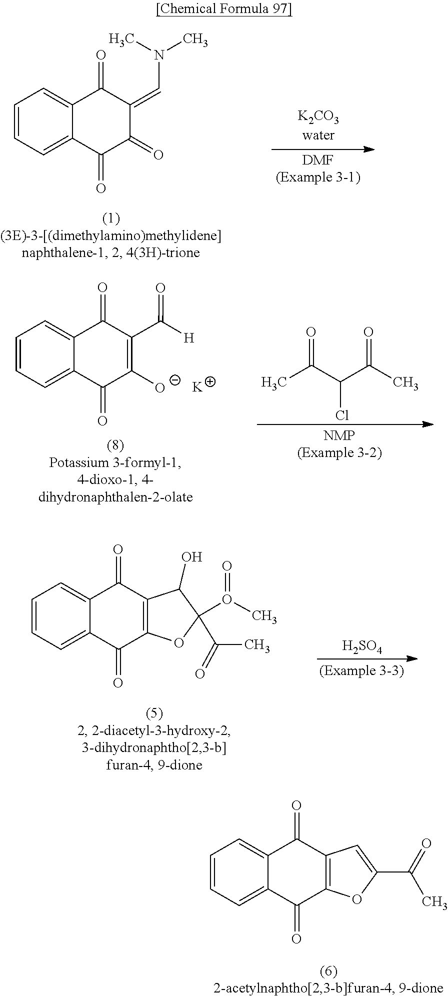

### Example 3-1: Manufacturing Method of potassium 3-formyl-1,4-dioxo-1,4-dihydronaphthalen-2-olate

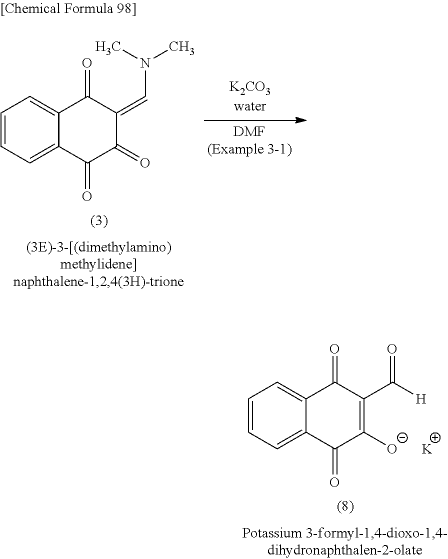 [SMILES: CN(C)/C=C1/C(=O)C(=O)c2ccccc2C1=O ; O=CC1=C([O-])C(=O)c2ccccc2C1=O ; [K+]]

Under a nitrogen atmosphere, an aqueous (16.4 g) solution of potassium carbonate (2.54 g) was added dropwise at room temperature to an NMP (127 mL) solution of the (3E)-3-[(dimethylamino)methylidene]naphthalene-1,2,4(3H)-trione (4.22 g) obtained in Example 1-1 described above. The compound of interest was eluted during the addition. After stirring for 3 hours at room temperature, the eluted solid was filtered and washed with water until the filtrate had a yellowish tinge, which was dried under reduced pressure at 50° C. to obtain potassium 3-formyl-1,4-dioxo-1,4-dihydronaphthalen-2-olate (4.18 g, yield: 95%, HPLC purity: 98.89 area %).

¹H-NMR (400 MHz, DMSO-d₆CDCl₃) δ: 10.05 (s, 1H), 8.03 (dd, 1H, J=7.3, 1.2 Hz), 7.84 (dd, 1H, J=7.3, 1.2 Hz), 7.76 (ddd, 1H, J=7.3, 7.3, 1.2 Hz), 7.63 (ddd, 1H, J=7.3, 7.3, 1.2 Hz).

### Example 3-2: Manufacture of 2,2-diacetyl-3-hydroxy-2,3-dihydronaphtho[2,3-b]furan-4,9-dione

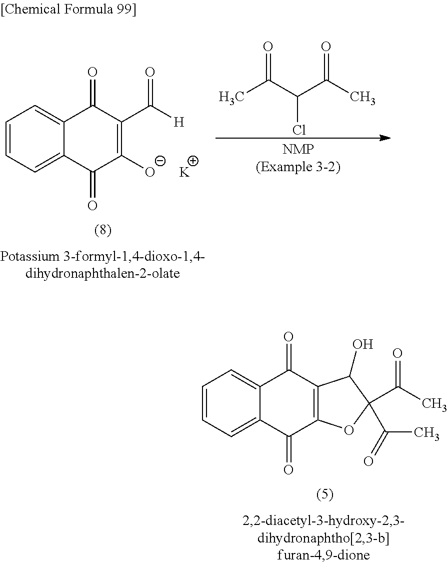

Under a nitrogen atmosphere, 3-chloropentane-2,4-dione (0.78 g) was added at room temperature to an NMP (8 g) solution of potassium 3-formyl-1,4-dioxo-1,4-dihydronaphthalen-2-olate (1.0 g). The mixture was stirred for 14 hours at 50° C. After cooling to room temperature, water (30 mL) and ethyl acetate (30 mL) were added, and the organic layer was separated. The resulting organic layer was further washed with water (30 mL) and saturated saline (20 g), dried with sodium sulfate, and then filtered and concentrated under reduced pressure at 40° C. to obtain 2,2-diacetyl-3-hydroxy-2,3-dihydronaphtho[2,3-b]furan-4,9-dione (1.39 g, yield: quant., HPLC purity: 86.97 area %, 2,2-diacetyl-3-hydroxy-2,3-dihydronaphtho[2,3-b]furan-4,9-dione isomer (2,2-diacetyl-3-hydroxy-2,3-dihydronaphtho[1,2-b]furan-4,5-dione): 12.73 area %, 2-acetylnaphtho[2,3-b]furan-4,9-dione: 0.30 area %)).

### Example 3-3: Manufacture of 2-acetylnaphtho[2,3-b]furan-4,9-dione

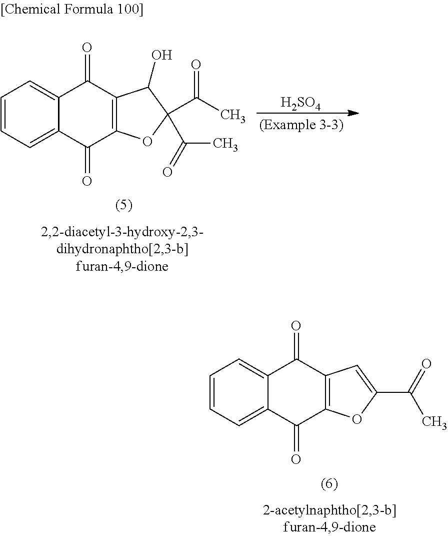 [SMILES: CC(=O)C1(C(C)=O)OC2=C(C(=O)c3ccccc3C2=O)C1O ; "H2SO4" ; CC(=O)c1cc2c(o1)C(=O)c1ccccc1C2=O]

2-acetylnaphtho[2,3-b]furan-4,9-dione was obtained in the same manner as Example 1-3 described above.

### Example 4: Manufacturing method of 2-acetylnaphtho[2,3-b]furan-4,9-dione

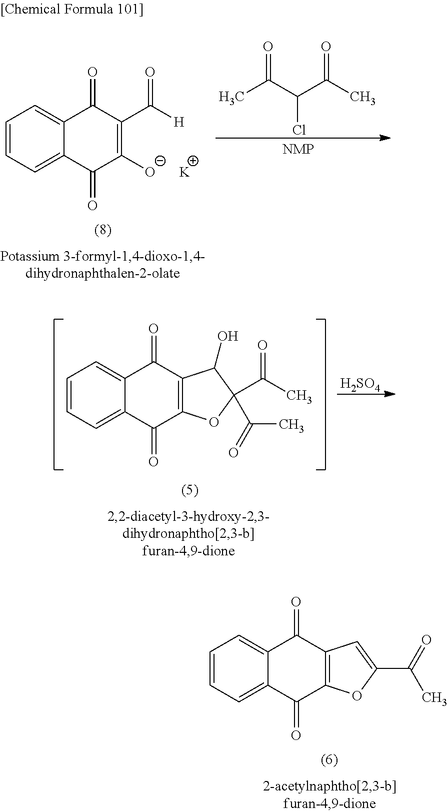

Under a nitrogen atmosphere, 3-chloropentane-2,4-dione (0.86 g) was added at room temperature to an NMP (8 g) solution of potassium 3-formyl-1,4-dioxo-1,4-dihydronaphthalen-2-olate (1.03 g) obtained in Example 3-1 described above. The mixture was stirred for 8 hours at 60° C. to prepare 2,2-diacetyl-3-hydroxy-2,3-dihydronaphtho[2,3-b]furan-4,9-dione. After cooling at room temperature, concentrated sulfuric acid (1.14 g) was added. The temperature was raised to 90° C. The mixture was stirred for 18 hours at 90° C. Water (0.7 mL) was added at 90° C. After stirring for 0.5 hours at 90° C., the mixture was cooled to 60° C. Methanol (4.2 mL) was added. The mixture was cooled to 20° C. and stirred for 3 hours at 20° C. The eluted solid was filtered. The solid was washed with methanol and water and dried under reduced pressure at 50° C. to obtain 2-acetylnaphtho[2,3-b]furan-4,9-dione (0.80 g, yield: 78%, HPLC purity: 99.81 area %).

Aggregate yield based on 2-hydroxy-1,4-naphthoquinone: 52%

### Example 5: Manufacturing Method of 2-acetylnaphtho[2,3-b]furan-4,9-dione

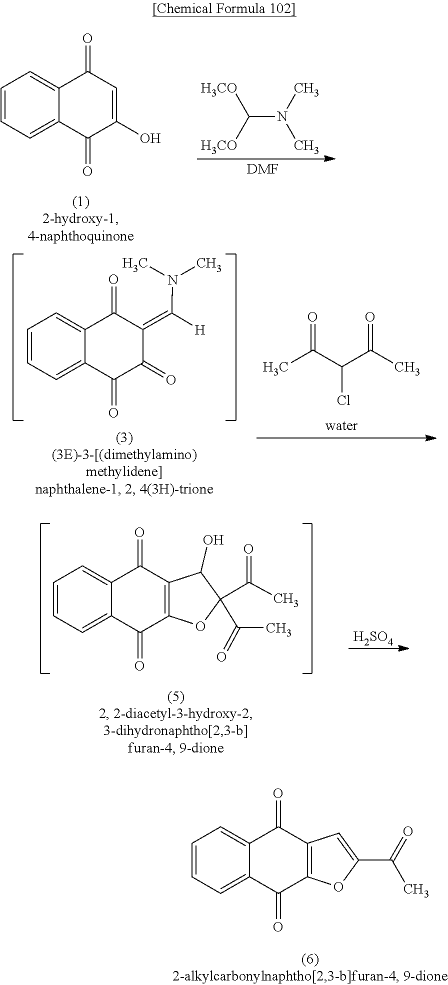

Under a nitrogen atmosphere, a DMF (45.0 g) solution of 2-hydroxy-1,4-naphthoquinone (10.0 g) was added dropwise at 0±5C to a DMF (30.0 g) solution of N,N-dimethylformamide dimethyl acetal (10.26 g). The mixture was stirred for 2 hours at 0±5° C. to prepare (3E)-3-[(dimethylamino)methylidene]naphthalene-1,2,4(3H)-trione. 3-chloropentane-2,4-dione (10.8 g) and water (10.3 g) were added at 0° C. to the reaction solution. The mixture was stirred for 5 hours at 50±5° C. to prepare 2,2-diacetyl-3-hydroxy-2,3-dihydronaphtho[2,3-b]furan-4,9-dione. After cooling to room temperature and adding sulfuric acid (14.6 g), the mixture was stirred for 3 hours at 60° C. and 9 hours at 90° C. After cooling to room temperature, methanol (40.0 g) was added. The mixture was stirred for 3 hours at room temperature, and the eluted solid was filtered. The solid was washed with methanol (52.7 g) and dried under reduced pressure at 50° C. to obtain 2-acetylnaphtho[2,3-b]furan-4,9-dione (4.23 g, yield: 31%, HPLC purity: 99.43 area %). Aggregate yield based on 2-hydroxy-1,4-naphthoquinone: 31%

### Example 6: Manufacturing Method of potassium 3-formyl-1,4-dioxo-1,4-dihydronaphthalen-2-olate

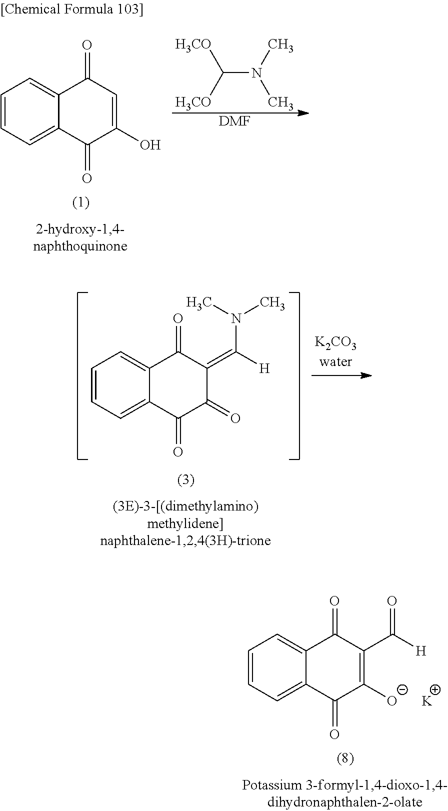

Under a nitrogen atmosphere, a DMF (40.0 g) of 2-hydroxy-1,4-naphthoquinone (10.0 g) was added dropwise at 0±5° C. to a DMF (30.0 g) solution of N,N-dimethylformamide dimethyl acetal (10.26 g). The mixture was stirred for 2 hours at 0±5° C. to prepare (3E)-3-[(dimethylamino)methylidene]naphthalene-1,2,4(3H)-trione. An aqueous solution prepared by dissolving potassium carbonate (8.7 g) into water (10.3 g) was added to the reaction solution at 5±5° C. The temperature was raised to 20±5° C., and the mixture was stirred for 1 to 19 hours. The eluted solid was filtered and washed with water until the filtrate had a yellowish tinge, which was dried under reduced pressure at 50° C. to obtain potassium 3-formyl-1,4-dioxo-1,4-dihydronaphthalen-2-olate (10.6 g, yield: 76%, HPLC purity: 98.36 area %).

### Comparative Example 1: Manufacture of 2-acetyl-2,3-dihydronaphtho[2,3-b]furan-4,9-dione and 2-acetylnaphtho[2,3-b]furan-4,9-dione

2-acetylnaphtho[2,3-b]furan-4,9-dione was manufactured in accordance with the method described in International Publication No. WO 2012/119265 as a Comparative Example, specifically as follows.

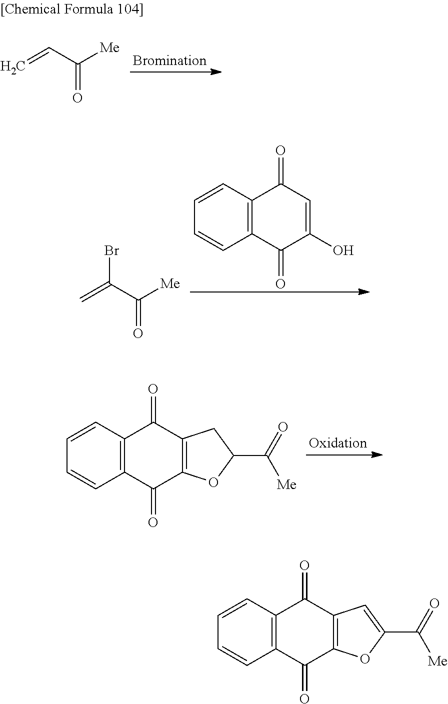

After adding 3-buten-2-one (16.1 g) to a 300 mL flask containing dichloromethane (40 mL) cooled to −2° C., bromine (36.7 g) was added dropwise over 25 minutes at 2 to 3° C. The reaction solution was washed with water (50 mL), and then the organic layer was dried with anhydrous sodium sulfate (5 g). After removing the anhydrous sodium sulfate, the organic layer was concentrated under reduced pressure. The resulting residue (48.8 g) was transferred to a 1 L flask using DMF (40 mL) and cooled to −2° C. After adding DBU (27.3 g) to a DMF solution dropwise over 15 minutes, DMF (50 mL) and 2-hydroxy-1,4-naphthoquinone (31.4 g) were added. The temperature was raised to room temperature under air atmosphere. DBU (25.8 g) was added dropwise to the reaction solution over 45 minutes at room temperature, and then DMF (50 mL) was added. The reaction solution was stirred for about 3 hours at room temperature and then cooled to 0° C. Water (500 mL) was added. The eluted compound was filtered out and washed with water (80 mL), aqueous 5% sodium carbonate solution (80 mL), water (80 mL), aqueous 2% acetic acid solution (80 mL), and ethanol (80 mL) in this order to obtain 2-acetyl-2,3-dihydronaphtho[2,3-b]furan-4,9-dione (21.1 g) (yield: 48%).

2-acetyl-2,3-dihydronaphtho[2,3-b]furan-4,9-dione (10.0 g), ethanol (250 mL), and DBU (5.1 g) were added to a 500 mL flask. The mixture was refluxed under heating for 30 minutes under an air atmosphere. The reaction solution was cooled to 0° C. and then water (250 mL) was added. The eluted crystal was obtained by filtration. The crystal was washed with water (10 mL), aqueous 2% acetic acid solution (10 mL), and ethanol (10 mL) in this order and dried under reduced pressure to obtain a crystal of 2-acetylnaphtho[2,3-b]furan-4,9-dione (3.2 g, HPLC purity: 99.70 area %) (yield of 32%).

Aggregate yield based on 2-hydroxy-1,4-naphthoquinone: 15%

Compared to Comparative Example 1, the method of the present disclosure dramatically improves the aggregate yield based on 2-hydroxy-1,4-naphthoquinone (e.g., improved 36% in Example 2), reduces the number of steps to 2 steps, and avoids the use of 3-buten-2-one, which has a risk of genotoxicity, and can manufacture highly pure 2-acetylnaphtho[2,3-b]furan-4,9-dione conveniently and efficiently without use of an ultralow temperature facility, separation, or concentration step from a safer and more cost efficient raw material.

As described above, the present disclosure is exemplified by the use of its preferred embodiments. However, it is understood that the scope of the present disclosure should be interpreted based solely on the Claims. The present application claims priority to Japanese Patent Application No. 2019-110987 (filed on Jun. 14, 2019) with the Japan Patent Office. The entire content thereof is incorporated herein by reference. It is also understood that any patent, any patent application, and any references cited herein should be incorporated herein by reference in the same manner as the contents are specifically described herein.

### INDUSTRIAL APPLICABILITY

A related substance of 2-alkylcarbonylnaphtho[2,3-b]furan-4,9-dione, which is useful as a pharmaceutical product, can be manufactured safely and cost-effectively at a high yield and high purity by using the manufacturing method of the present disclosure.

1. A manufacturing method of a compound or a pharmaceutically acceptable salt thereof represented by formula (6) 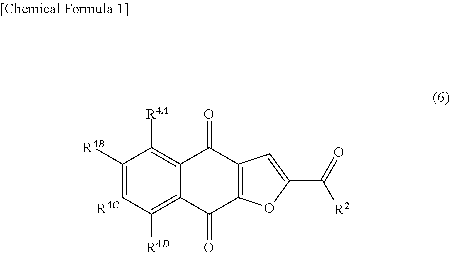
or a solvate thereof, wherein
R² is a hydrogen atom, an optionally substituted C₁₋₁₀ alkyl group, an optionally substituted C₃₋₁₀ cycloalkyl group, an optionally substituted C₆₋₁₀ aryl group, an optionally substituted 3- to 12-membered monocyclic or polycyclic heterocyclic group, and
R^{4A}, R^{4B}, R^{4C}, and R^{4D} are the same or different, each independently a hydrogen atom, a halogen atom, a cyano group, a hydroxyl group, an optionally substituted amino group, an optionally substituted C₁₋₆ alkyl group, an optionally substituted C₃₋₁₀ cycloalkyl group, an optionally substituted C₆₋₁₀ aryl group, an optionally substituted 3- to 12-membered monocyclic or polycyclic heterocyclic group, an optionally substituted C₁₋₆ alkoxy group, an optionally substituted C₃₋₁₀ cycloalkoxy group, an optionally substituted C₆₋₁₀ aryloxy group, an optionally substituted 3- to 12-membered monocyclic or polycyclic heterocyclic oxy group, a carboxyl group, an optionally substituted C₁₋₆ alkylcarbonyl group, an optionally substituted C₃₋₁₀ cycloalkylcarbonyl group, an optionally substituted C₆₋₁₀ arylcarbonyl group, an optionally substituted 3- to 12-membered monocyclic or polycyclic heterocyclic carbonyl group, an optionally substituted C₁₋₆ alkoxycarbonyl group, an optionally substituted C₃₋₁₀ cycloalkoxycarbonyl group, an optionally substituted C₆₋₁₀ aryloxycarbonyl group, an optionally substituted 3- to 12-membered monocyclic or polycyclic heterocyclic oxycarbonyl group, an optionally substituted aminocarbonyl group, an optionally substituted C₁₋₆ alkylthio group, an optionally substituted C₃₋₁₀ cycloalkylthio group, an optionally substituted C₆₋₁₀ arylthio group, an optionally substituted 3- to 12-membered monocyclic or polycyclic heterocyclic thio group, a sulfinic acid group, an optionally substituted C₁₋₆ alkylsulfinyl group, an optionally substituted C₃₋₁₀ cycloalkylsulfinyl group, an optionally substituted C₆₋₁₀ arylsulfinyl group, an optionally substituted 3- to 12-membered monocyclic or polycyclic heterocyclic sulfinyl group, an optionally substituted aminosulfinyl group, a sulfonic acid group, an optionally substituted C₁₋₆ alkylsulfonyl group, an optionally substituted C₃₋₁₀ cycloalkylsulfonyl group, an optionally substituted C₆₋₁₀ arylsulfonyl group, an optionally substituted 3- to 12-membered monocyclic or polycyclic heterocyclic sulfonyl group, or an optionally substituted aminosulfonyl group, comprising the following step (b):
(b) reacting a compound or a pharmaceutically acceptable salt thereof represented by formula (3) 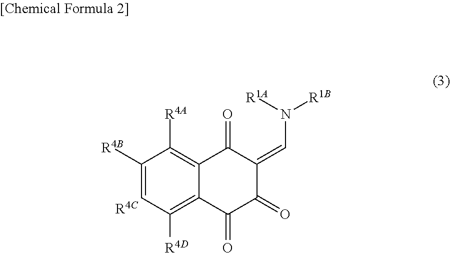
or a solvate thereof, wherein
R^{1A} and R^{1B} are the same or different, each independently a hydrogen atom, an optionally substituted C₁₋₁₀ alkyl, or an optionally substituted C₃₋₁₀ cycloalkyl group, wherein R^{1A} and R^{1B} are not simultaneously a hydrogen atom,
with a compound or a pharmaceutically acceptable salt thereof represented by formula (4) 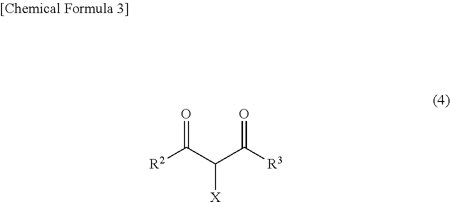
or a solvate thereof, wherein
R² is defined as the same as above,
R³ is a hydrogen atom, an optionally substituted C₁₋₁₀ alkyl group, an optionally substituted C₃₋₁₀ cycloalkyl group, an optionally substituted C₆₋₁₀ aryl group, an optionally substituted 3- to 12-membered monocyclic or polycyclic heterocyclic group, and
X is a halogen atom,
in the presence of a solvent to manufacture a compound or a pharmaceutically acceptable salt thereof represented by formula (5) 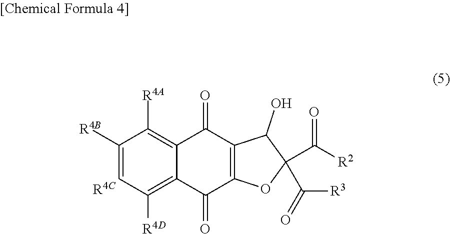
or a solvate thereof,
wherein R², R³, R^{4A}, R^{4B}, R^{4C}, and R^{4D} are defined as the same as above.

2. The manufacturing method of claim 1, wherein water is added in step (b).

3. The manufacturing method of claim 2, wherein an amount of water added is 1.0 equivalent to 40.0 equivalent with respect to a compound represented by formula (3) in step (b).

4. The manufacturing method of claim 1, wherein an amount of a compound represented by formula (4) used is 1.0 equivalent to 4.0 equivalent with respect to a compound represented by formula (3) in step (b).

5. The manufacturing method of claim 1, wherein the solvent is an amide-based solvent in step (b).

6. The manufacturing method of claim 1, wherein the solvent is an N-methyl-2-pyrrolidone in step (b).

7. The manufacturing method of claim 1, wherein a reaction temperature is 10° C. to 70° C. in step (b).

8. The manufacturing method of claim 1, further comprising the following step (c) after step (b):
(c) heating a compound or a pharmaceutically acceptable salt thereof represented by formula (5) 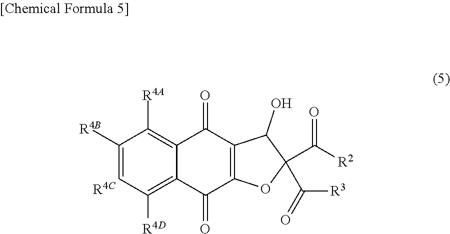
obtained in step (b) or a solvate thereof, wherein R², R³, R^{4A}, R^{4B}, R^{4C}, and R^{4D} are defined as the same as above, in the presence of a solvent to manufacture a compound or a pharmaceutically acceptable salt thereof represented by formula (6) 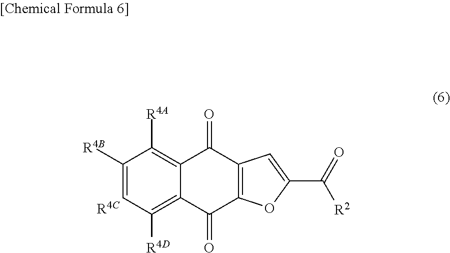
or a solvate thereof, wherein R², R^{4A}, R^{4B}, R^{4C}, and R^{4D} are defined as the same as above.

9. The manufacturing method of claim 8, wherein an acid is added in step (c).

10. The manufacturing method of claim 9, wherein the acid is sulfuric acid or a hydrochloric acid.

11. The manufacturing method of claim 9, wherein an amount of the acid added is 1.0 equivalent to 4.0 equivalent with respect to a compound represented by formula (5) in step (c).

12. The manufacturing method of claim 8, wherein a reaction temperature in step (c) is 20° C. to 150° C.

13. The manufacturing method of claim 8, wherein a compound represented by formula (6) is deposited after completion of a reaction in step (c).

14. The manufacturing method of claim 8, wherein step (c) is performed without isolating the compound or pharmaceutically acceptable salt thereof represented by formula (5) or a solvate thereof in step (b).

15. The manufacturing method of claim 1, further comprising the following step (a) before step (b):
(a) reacting 2-hydroxy-1,4-naphthoquinone or a pharmaceutically acceptable salt thereof represented by formula (1) 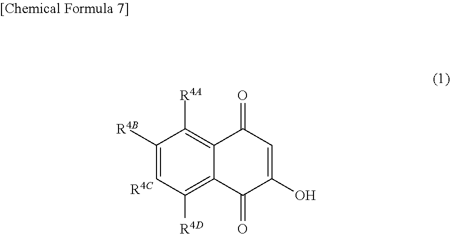
or a solvate thereof, wherein R^{4A}, R^{4B}, R^{4C}, and R^{4D} are defined as the same as above, with a compound or a pharmaceutically acceptable salt thereof represented by formula (2a) or (2b) 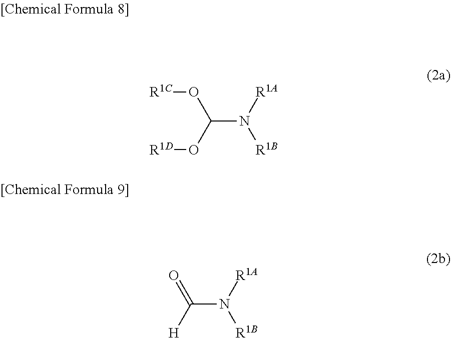
or a solvate thereof, wherein
R^{1A} and R^{1B} are defined as the same as above, and
R^{1C} and R^{1D} are the same or different, each independently a hydrogen atom, an optionally substituted C₁₋₆ alkyl group, an optionally substituted 3- to 12-membered monocyclic or polycyclic heterocyclic group,
in the presence of a solvent to manufacture a compound or a pharmaceutically acceptable salt thereof represented by formula (3) 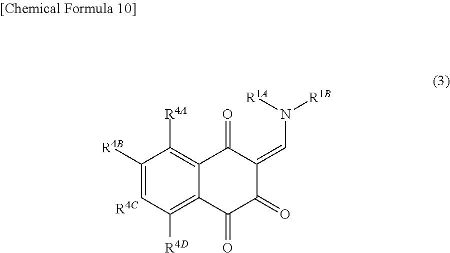
or a solvate thereof, wherein R^{1A}, R^{1B}, R^{4A}, R^{4B}, R^{4C}, and R^{4D} are defined as the same as above.

16. The manufacturing method of claim 15, wherein the solvent is an amide-based solvent in step (a).

17. The manufacturing method of claim 15, wherein the solvent is N,N-dimethylformamide or N-methyl-2-pyrrolidone in step (a).

18. The manufacturing method of claim 15, wherein a reaction temperature is −10° C. to 30° C. in step (a).

19. The manufacturing method of claim 15, wherein an amount of a compound represented by formula (2a) or (2b) used is 1.0 equivalent to 10.0 equivalent with respect to a compound represented by formula (1) in step (a).

20. The manufacturing method of claim 15, wherein a compound represented by formula (2a) is used in step (a).

21. The manufacturing method of claim 15, wherein a compound represented by formula (3) is deposited after completion of a reaction in step (a).

22. The manufacturing method of claim 21, wherein a solid that is the deposited compound represented by formula (3) is filtered out and washed with an alcohol-based solvent in step (a).

23. The manufacturing method of claim 22, wherein the alcohol-based solvent is methanol.

24. The manufacturing method of claim 15, wherein steps (a), (b), and (c) are performed without isolating the compounds represented by formula (3) and formula (5) in steps (a), (b), and (c).

25. The manufacturing method of claim 1, wherein R^{1A} and R^{1B} are methyl groups.

26. The manufacturing method of claim 15, wherein R^{1C} and R^{1D} are methyl groups.

27. The manufacturing method of claim 1, wherein R² is a methyl group.

28. The manufacturing method of claim 1, wherein R³ is a methyl group.

29. The manufacturing method of claim 1, wherein R^{4A}, R^{4B}, R^{4C}, and R^{4D} are hydrogen atoms.

30. A manufacturing method of a compound or a pharmaceutically acceptable salt thereof represented by formula (3) 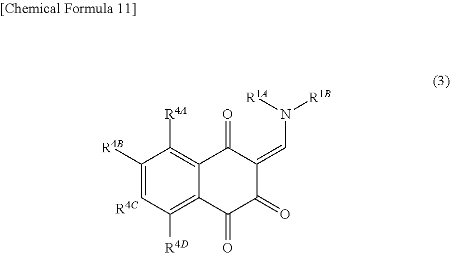
or a solvate thereof, wherein
R^{1A} and R^{1B} are the same or different, each independently a hydrogen atom, an optionally substituted C₁₋₁₀ alkyl group, or an optionally substituted C₃₋₁₀ cycloalkyl group, wherein R^{1A} and R^{1B} are not simultaneously a hydrogen atom, and
R^{4A}, R^{4B}, R^{4C}, and R^{4D} are the same or different, each independently a hydrogen atom, a halogen atom, a cyano group, a hydroxyl group, an optionally substituted amino group, an optionally substituted C₁₋₆ alkyl group, an optionally substituted C₃₋₁₀ cycloalkyl group, an optionally substituted C₆₋₁₀ aryl group, an optionally substituted 3- to 12-membered monocyclic or polycyclic heterocyclic group, an optionally substituted C₁₋₆ alkoxy group, an optionally substituted C₃₋₁₀ cycloalkoxy group, an optionally substituted C₆₋₁₀ aryloxy group, an optionally substituted 3- to 12-membered monocyclic or polycyclic heterocyclic oxy group, a carboxyl group, an optionally substituted C₁₋₆ alkylcarbonyl group, an optionally substituted C₃₋₁₀ cycloalkylcarbonyl group, an optionally substituted C₆₋₁₀ arylcarbonyl group, an optionally substituted 3- to 12-membered monocyclic or polycyclic heterocyclic carbonyl group, an optionally substituted C₁₋₆ alkoxycarbonyl group, an optionally substituted C₃₋₁₀ cycloalkoxycarbonyl group, an optionally substituted C₆₋₁₀ aryloxycarbonyl group, an optionally substituted 3- to 12-membered monocyclic or polycyclic heterocyclic oxycarbonyl group, an optionally substituted aminocarbonyl group, an optionally substituted C₁₋₆ alkylthio group, an optionally substituted C₃₋₁₀ cycloalkylthio group, an optionally substituted C₆₋₁₀ arylthio group, an optionally substituted 3- to 12-membered monocyclic or polycyclic heterocyclic thio group, a sulfinic acid group, an optionally substituted C₁₋₆ alkylsulfinyl group, an optionally substituted C₃₋₁₀ cycloalkylsulfinyl group, an optionally substituted C₆₋₁₀ arylsulfinyl group, an optionally substituted 3- to 12-membered monocyclic or polycyclic heterocyclic sulfinyl group, an optionally substituted aminosulfinyl group, a sulfonic acid group, an optionally substituted C₁₋₆ alkylsulfonyl group, an optionally substituted C₃₋₁₀ cycloalkylsulfonyl group, an optionally substituted C₆₋₁₀ arylsulfonyl group, an optionally substituted 3- to 12-membered monocyclic or polycyclic heterocyclic sulfonyl group, or an optionally substituted aminosulfonyl group, comprising the following step (a):
(a) reacting 2-hydroxy-1,4-naphthoquinone or a pharmaceutically acceptable salt thereof represented by formula (1) 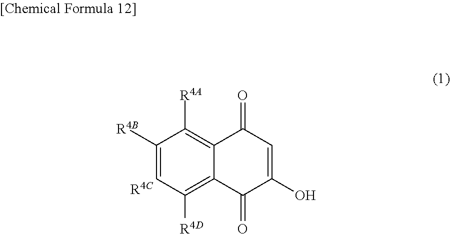
or a solvate thereof, wherein R^{4A}, R^{4B}, R^{4C}, and R^{4D} are defined as the same as above, with a compound or a pharmaceutically acceptable salt thereof represented by formula (2a) or (2b) 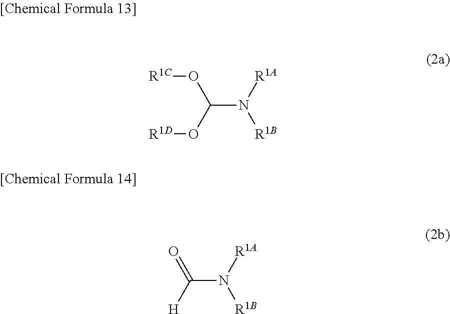
or a solvate thereof, wherein
R^{1A} and R^{1B} are defined as the same as above, and
R^{1C} and R^{1D} are the same or different, each independently a hydrogen atom, an optionally substituted C₁₋₆ alkyl group, an optionally substituted 3- to 12-membered monocyclic or polycyclic heterocyclic group,
in the presence of a solvent to manufacture a compound or a pharmaceutically acceptable salt thereof represented by formula (3) 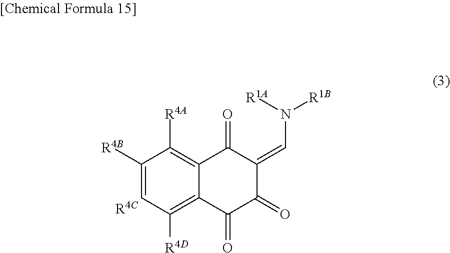
or a solvate thereof,
wherein R^{1A}, R^{1B}, R^{4A}, R^{4B}, R^{4C}, and R^{4D} are defined as the same as above.

31. The manufacturing method of claim 30, wherein the solvent is an amide-based solvent in step (a).

32. The manufacturing method of claim 30, wherein the solvent is N,N-dimethylformamide or N-methyl-2-pyrrolidone in step (a).

33. The manufacturing method of claim 30, wherein a reaction temperature is −10° C. to 30° C. in step (a).

34. The manufacturing method of claim 30, wherein an amount of a compound represented by formula (2a) or (2b) used is 1.0 equivalent to 10.0 equivalent with respect to a compound represented by formula (1) in step (a).

35. The manufacturing method of claim 30, wherein a compound represented by formula (2a) is used in step (a).

36. The manufacturing method of claim 30, wherein a compound represented by formula (3) is deposited after completion of a reaction in step (a).

37. The manufacturing method of claim 36, wherein a solid that is the deposited compound represented by formula (3) is filtered out and washed with an alcohol-based solvent in step (a).

38. The manufacturing method of claim 37, wherein the alcohol-based solvent is methanol.

39. The manufacturing method of claim 30, wherein R^{1A} and R^{1B} are methyl groups.

40. The manufacturing method of claim 30, wherein R^{1C} and R^{1D} are methyl groups.

41. The manufacturing method of claim 30, wherein R^{4A}, R^{4B}, R^{4C}, and R^{4D} are hydrogen atoms.

42. A manufacturing method of a compound or a pharmaceutically acceptable salt thereof represented by formula (5) 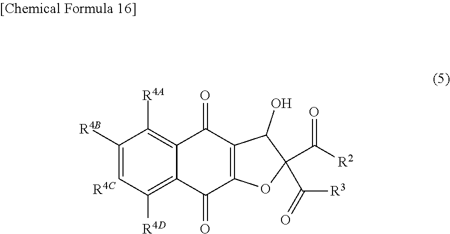
or a solvate thereof, wherein
R² is a hydrogen atom, an optionally substituted C₁₋₁₀ alkyl group, an optionally substituted C₃₋₁₀ cycloalkyl group, an optionally substituted C₆₋₁₀ aryl group, or an optionally substituted 3- to 12-membered monocyclic or polycyclic heterocyclic group,
R³ is a hydrogen atom, an optionally substituted C₁₋₁₀ alkyl group, an optionally substituted C₃₋₁₀ cycloalkyl group, an optionally substituted C₆₋₁₀ aryl group, or an optionally substituted 3- to 12-membered monocyclic or polycyclic heterocyclic group, and
R^{4A}, R^{4B}, R^{4C}, and R^{4D} are the same or different, each independently a hydrogen atom, a halogen atom, a cyano group, a hydroxyl group, an optionally substituted amino group, an optionally substituted C₁₋₆ alkyl group, an optionally substituted C₃₋₁₀ cycloalkyl group, an optionally substituted C₆₋₁₀ aryl group, an optionally substituted 3- to 12-membered monocyclic or polycyclic heterocyclic group, an optionally substituted C₁₋₆ alkoxy group, an optionally substituted C₃₋₁₀ cycloalkoxy group, an optionally substituted C₆₋₁₀ aryloxy group, an optionally substituted 3- to 12-membered monocyclic or polycyclic heterocyclic oxy group, a carboxyl group, an optionally substituted C₁₋₆ alkylcarbonyl group, an optionally substituted C₃₋₁₀ cycloalkylcarbonyl group, an optionally substituted C₆₋₁₀ arylcarbonyl group, an optionally substituted 3- to 12-membered monocyclic or polycyclic heterocyclic carbonyl group, an optionally substituted C₁₋₆ alkoxycarbonyl group, an optionally substituted C₃₋₁₀ cycloalkoxycarbonyl group, an optionally substituted C₆₋₁₀ aryloxycarbonyl group, an optionally substituted 3- to 12-membered monocyclic or polycyclic heterocyclic oxycarbonyl group, an optionally substituted aminocarbonyl group, an optionally substituted C₁₋₆ alkylthio group, an optionally substituted C₃₋₁₀ cycloalkylthio group, an optionally substituted C₆₋₁₀ arylthio group, an optionally substituted 3- to 12-membered monocyclic or polycyclic heterocyclic thio group, a sulfinic acid group, an optionally substituted C₁₋₆ alkylsulfinyl group, an optionally substituted C₃₋₁₀ cycloalkylsulfinyl group, an optionally substituted C₆₋₁₀ arylsulfinyl group, an optionally substituted 3- to 12-membered monocyclic or polycyclic heterocyclic sulfinyl group, an optionally substituted aminosulfinyl group, a sulfonic acid group, an optionally substituted C₁₋₆ alkylsulfonyl group, an optionally substituted C₃₋₁₀ cycloalkylsulfonyl group, an optionally substituted C₆₋₁₀ arylsulfonyl group, an optionally substituted 3- to 12-membered monocyclic or polycyclic heterocyclic sulfonyl group, or an optionally substituted aminosulfonyl group, comprising the following step (b):
(b) reacting a compound or a pharmaceutically acceptable salt thereof represented by formula (3) 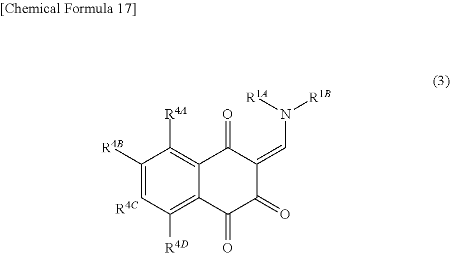
or a solvate thereof,
wherein R^{1A} and R^{1B} are the same or different, each independently a hydrogen atom, an optionally substituted C₁₋₁₀ alkyl, or an optionally substituted C₃₋₁₀ cycloalkyl group, wherein R^{1A} and R^{1B} are not simultaneously a hydrogen atom,
with a compound or a pharmaceutically acceptable salt thereof represented by formula (4) 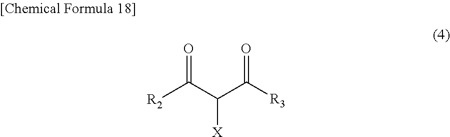
or a solvate thereof, wherein R² and R³ are defined as the same as above, and X is a halogen atom,
in the presence of a solvent to manufacture a compound or a pharmaceutically acceptable salt thereof represented by formula (5) 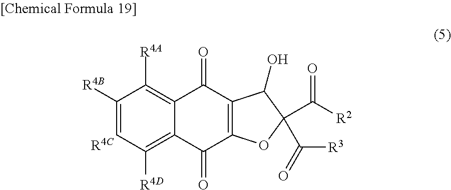
or a solvate thereof,
wherein R², R³, R^{4A}, R^{4B}, R^{4C}, and R^{4D} are defined as the same as above.

43. The manufacturing method of claim 42, wherein water is added in step (b).

44. The manufacturing method of claim 43, wherein an amount of water added is 1.0 equivalent to 40.0 equivalent with respect to a compound represented by formula (3) in step (b).

45. The manufacturing method of claim 42, wherein an amount of a compound represented by formula (4) used is 1.0 equivalent to 4.0 equivalent with respect to a compound represented by formula (3) in step (b).

46. The manufacturing method of claim 42, wherein the solvent is an amide-based solvent in step (b).

47. The manufacturing method of claim 42, wherein the solvent is an N-methyl-2-pyrrolidone in step (b).

48. The manufacturing method of claim 42, wherein a reaction temperature is 10° C. to 70° C. in step (b).

49. The manufacturing method of claim 42, further comprising the following step (a) before step (b):
(a) reacting 2-hydroxy-1,4-naphthoquinone or a pharmaceutically acceptable salt thereof represented by formula (1) 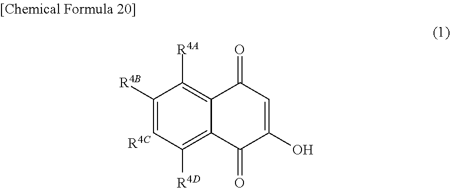
or a solvate thereof, wherein R^{4A}, R^{4B}, R^{4C}, and R^{4D} are defined as the same as above, with a compound or a pharmaceutically acceptable salt thereof represented by formula (2a) or (2b) 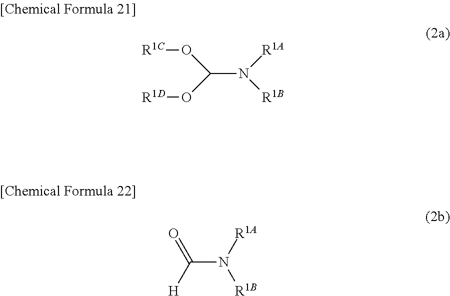
or a solvate thereof, wherein R^{1A} and R^{1B} are defined as the same as above, and R^{1C} and R^{ID} are the same or different, each independently a hydrogen atom, an optionally substituted C₁₋₆ alkyl group, or an optionally substituted 3- to 12-membered monocyclic or polycyclic heterocyclic group,
in the presence of a solvent to manufacture a compound or a pharmaceutically acceptable salt thereof represented by formula (3) 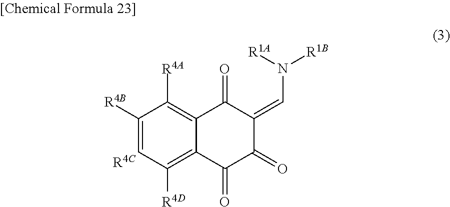
or a solvate thereof, wherein R^{1A}, R^{1B}, R^{4A}, R^{4B}, R^{4C}, and R^{4D} are defined as the same as above.

50. The manufacturing method of claim 49, wherein the solvent is an amide-based solvent in step (a).

51. The manufacturing method of claim 49, wherein the solvent is N,N-dimethylformamide or N-methyl-2-pyrrolidone in step (a).

52. The manufacturing method of claim 49, wherein a reaction temperature is −10° C. to 30° C. in step (a).

53. The manufacturing method of claim 49, wherein an amount of a compound represented by formula (2a) or (2b) used is 1.0 equivalent to 10.0 equivalent with respect to a compound represented by formula (1) in step (a).

54. The manufacturing method of claim 49, wherein a compound represented by formula (2a) is used in step (a).

55. The manufacturing method of claim 49, wherein a compound represented by formula (3) is deposited after completion of a reaction in step (a).

56. The manufacturing method of claim 55, wherein a solid that is the deposited compound represented by formula (3) is filtered out and washed with an alcohol-based solvent in step (a).

57. The manufacturing method of claim 56, wherein the alcohol-based solvent is methanol.

58. The manufacturing method of claim 42, wherein R^{1A} and R^{1B} are methyl groups.

59. The manufacturing method of claim 49, wherein R^{1C} and R^{1D} are methyl groups.

60. The manufacturing method of claim 42, wherein R² is a methyl group.

61. The manufacturing method of claim 42, wherein R³ is a methyl group.

62. The manufacturing method of claim 42, wherein R^{4A}, R^{4B}, R^{4C}, and R^{4D} are hydrogen atoms.

63. A manufacturing method of a compound or a pharmaceutically acceptable salt thereof represented by formula (6) 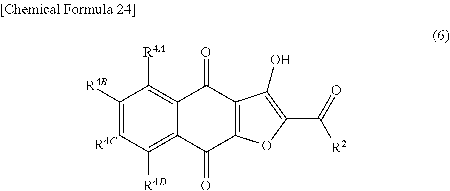
or a solvate thereof, wherein
R² is a hydrogen atom, an optionally substituted C₁₋₁₀ alkyl group, an optionally substituted C₃₋₁₀ cycloalkyl group, an optionally substituted C₆₋₁₀ aryl group, or an optionally substituted 3- to 12-membered monocyclic or polycyclic heterocyclic group, and
R^{4A}, R^{4B}, R^{4C}, and R^{4D} are the same or different, each independently a hydrogen atom, a halogen atom, a cyano group, a hydroxyl group, an optionally substituted amino group, an optionally substituted C₁₋₆ alkyl group, an optionally substituted C₃₋₁₀ cycloalkyl group, an optionally substituted C₆₋₁₀ aryl group, an optionally substituted 3- to 12-membered monocyclic or polycyclic heterocyclic group, an optionally substituted C₁₋₆ alkoxy group, an optionally substituted C₃₋₁₀ cycloalkoxy group, an optionally substituted C₆₋₁₀ aryloxy group, an optionally substituted 3- to 12-membered monocyclic or polycyclic heterocyclic oxy group, a carboxyl group, an optionally substituted C₁₋₆ alkylcarbonyl group, an optionally substituted C₃₋₁₀ cycloalkylcarbonyl group, an optionally substituted C₆₋₁₀ arylcarbonyl group, an optionally substituted 3- to 12-membered monocyclic or polycyclic heterocyclic carbonyl group, an optionally substituted C₁₋₆ alkoxycarbonyl group, an optionally substituted C₃₋₁₀ cycloalkoxycarbonyl group, an optionally substituted C₆₋₁₀ aryloxycarbonyl group, an optionally substituted 3- to 12-membered monocyclic or polycyclic heterocyclic oxycarbonyl group, an optionally substituted aminocarbonyl group, an optionally substituted C₁₋₆ alkylthio group, an optionally substituted C₃₋₁₀ cycloalkylthio group, an optionally substituted C₆₋₁₀ arylthio group, an optionally substituted 3- to 12-membered monocyclic or polycyclic heterocyclic thio group, a sulfinic acid group, an optionally substituted C₁₋₆ alkylsulfinyl group, an optionally substituted C₃₋₁₀ cycloalkylsulfinyl group, an optionally substituted C₆₋₁₀ arylsulfinyl group, an optionally substituted 3- to 12-membered monocyclic or polycyclic heterocyclic sulfinyl group, an optionally substituted aminosulfinyl group, a sulfonic acid group, an optionally substituted C₁₋₆ alkylsulfonyl group, an optionally substituted C₃₋₁₀ cycloalkylsulfonyl group, an optionally substituted C₆₋₁₀ arylsulfonyl group, an optionally substituted 3- to 12-membered monocyclic or polycyclic heterocyclic sulfonyl group, or an optionally substituted aminosulfonyl group, comprising the following step (e):
(e) reacting a compound or a pharmaceutically acceptable salt thereof represented by formula (8) 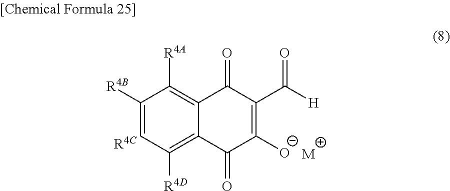
or a solvate thereof, wherein
M is Li, Na, or K, and
R^{4A}, R^{4B}, R^{4C}, and R^{4D} are defined as the same as above,
with a compound or a pharmaceutically acceptable salt thereof represented by formula (4) 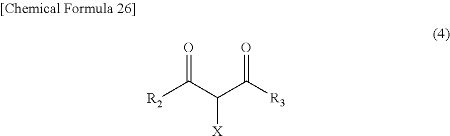
or a solvate thereof,
wherein
R² is defined as the same as above,
R³ is a hydrogen atom, an optionally substituted C₁₋₁₀ alkyl group, an optionally substituted C₃₋₁₀ cycloalkyl group, an optionally substituted C₆₋₁₀ aryl group, an optionally substituted 3- to 12-membered monocyclic or polycyclic heterocyclic group, and
X is a halogen atom
in the presence of a solvent to manufacture a compound or a pharmaceutically acceptable salt thereof represented by formula (5) 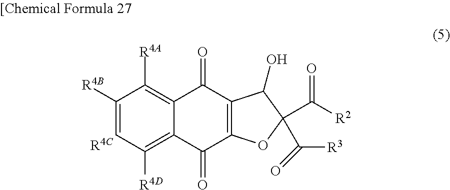
or a solvate thereof,
wherein R², R³, R^{4A}, R^{4B}, R^{4C}, and R^{4D} are defined as the same as above.

64. The manufacturing method of claim 63, wherein the solvent is an amide-based solvent in step (e).

65. The manufacturing method of claim 63, wherein the solvent in step (e) is an N-methyl-2-pyrrolidone.

66. The manufacturing method of claim 63, wherein an amount of a compound represented by formula (4) used is 1.0 equivalent to 4.0 equivalent with respect to a compound represented by formula (8) in step (e).

67. The manufacturing method of claim 63, wherein a reaction temperature is 10° C. to 70° C. in step (e).

68. The manufacturing method of claim 63, further comprising the following step (c) after step (e):
(c) heating a compound or a pharmaceutically acceptable salt thereof represented by formula (5) 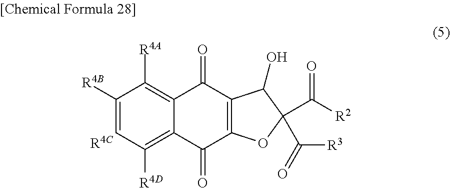
obtained in step (e), or a solvate thereof, wherein R², R³, R^{4A}, R^{4B}, R^{4C}, and R^{4D} are defined as the same as above, in the presence of a solvent to manufacture a compound or a pharmaceutically acceptable salt thereof represented by formula (6) 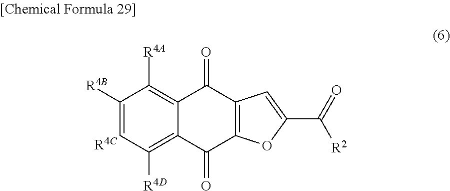
or a solvate thereof, wherein R² is defined as the same as above.

69. The manufacturing method of claim 68, wherein an acid is added in step (c).

70. The manufacturing method of claim 69, wherein the acid is sulfuric acid or a hydrochloric acid.

71. The manufacturing method of claim 69, wherein an amount of the acid added is 1.0 equivalent to 4.0 equivalent with respect to a compound represented by formula (5) in step (c).

72. The manufacturing method of claim 68, wherein a reaction temperature in step (c) is 20° C. to 150° C.

73. The manufacturing method of claim 68, wherein a compound represented by formula (6) is deposited after completion of a reaction in step (c).

74. The manufacturing method of claim 68, wherein step (c) is performed without isolating the compound or pharmaceutically acceptable salt thereof represented by formula (5) or a solvate thereof in step (e).

75. The manufacturing method of claim 68, further comprising the following step (d) before step (e):
(d) reacting a compound or a pharmaceutically acceptable salt thereof represented by formula (3) 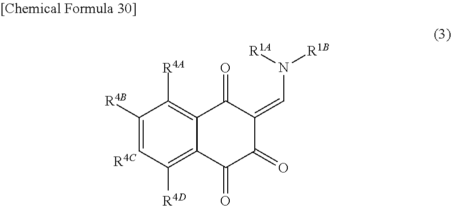
or a solvate thereof, wherein
R^{1A} and R^{1B} are the same or different, each independently a hydrogen atom, an optionally substituted C₁₋₁₀ alkyl, or an optionally substituted C₃₋₁₀ cycloalkyl group, wherein R^{1A} and R^{1B} are not simultaneously a hydrogen atom, and
R^{4A}, R^{4B}, R^{4C}, and R^{4D} are defined as the same as above, with a base in a solvent to manufacture a compound or a pharmaceutically acceptable salt thereof represented by formula (8) 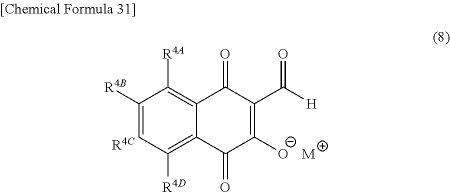
or a solvate thereof, wherein M, R^{4A}, R^{4B}, R^{4C}, and R^{4D} are defined as the same as above.

76. The manufacturing method of claim 75, wherein a reaction is performed in the presence of water in step (d).

77. The manufacturing method of claim 76, wherein an amount of water used is 0.1-fold to 10.0-fold in weight with respect to a compound represented by formula (3) in step (d).

78. The manufacturing method of claim 75, wherein the base is an inorganic base in step (d).

79. The manufacturing method of claim 78, wherein the inorganic base is potassium carbonate.

80. The manufacturing method of claim 75, wherein an amount of a base used is 1.0 equivalent to 10.0 equivalent with respect to a compound represented by formula (3) in step (d).

81. The manufacturing method of claim 75, wherein the solvent is an amide-based solvent in step (d).

82. The manufacturing method of claim 75, wherein the solvent is N,N-dimethylformamide or N-methyl-2-pyrrolidone in step (d).

83. The manufacturing method of claim 75, wherein a compound represented by formula (8) is deposited after completion of a reaction in step (d).

84. The manufacturing method of claim 75, wherein a reaction temperature is 0° C. to 50° C. in step (d).

85. The manufacturing method of claim 63, wherein R^{1A} and R^{1B} are methyl groups.

86. The manufacturing method of claim 63, wherein R² is a methyl group.

87. The manufacturing method of claim 63, wherein R³ is a methyl group.

88. The manufacturing method of claim 63, wherein R^{4A}, R^{4B}, R^{4C}, and R^{4D} are hydrogen atoms.

89. The manufacturing method of claim 63, wherein M is potassium.

90. A compound or a pharmaceutically acceptable salt thereof represented by formula (3) 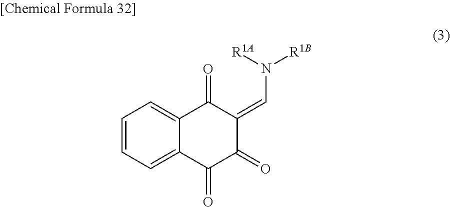
or a solvate thereof, wherein
R^{1A} and R^{1B} are the same or different, each independently a hydrogen atom, an optionally substituted C₁₋₁₀ alkyl, or an optionally substituted C₃₋₁₀ cycloalkyl group, wherein R^{1A} and R^{1B} are not simultaneously a hydrogen atom.

91. The compound or a pharmaceutically acceptable salt, or a solvate thereof of claim 90, wherein R^{1A} and R^{1B} are optionally substituted C₁₋₁₀ alkyl groups.

92. The compound or a pharmaceutically acceptable salt, or a solvate thereof of claim 90, wherein R^{1A} and R^{1B} are methyl groups.

93. A compound or a pharmaceutically acceptable salt thereof represented by formula (5) 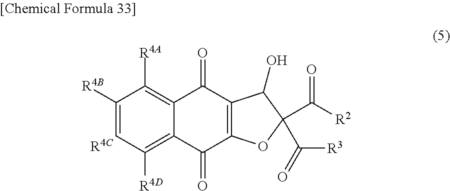
or a solvate thereof, wherein
R² is a hydrogen atom, an optionally substituted C₁₋₁₀ alkyl group, an optionally substituted C₃₋₁₀ cycloalkyl group, an optionally substituted C₆₋₁₀ aryl group, or an optionally substituted 3- to 12-membered monocyclic or polycyclic heterocyclic group,
R³ is a hydrogen atom, an optionally substituted C₁₋₁₀ alkyl group, an optionally substituted C₃₋₁₀ cycloalkyl group, an optionally substituted C₆₋₁₀ aryl group, or an optionally substituted 3- to 12-membered monocyclic or polycyclic heterocyclic group, and
R^{4A}, R^{4B}, R^{4C}, and R^{4D} are the same or different, each independently a hydrogen atom, a halogen atom, a cyano group, a hydroxyl group, an optionally substituted amino group, an optionally substituted C₁₋₆ alkyl group, an optionally substituted C₃₋₁₀ cycloalkyl group, an optionally substituted C₆₋₁₀ aryl group, an optionally substituted 3- to 12-membered monocyclic or polycyclic heterocyclic group, an optionally substituted C₁₋₆ alkoxy group, an optionally substituted C₃₋₁₀ cycloalkoxy group, an optionally substituted C₆₋₁₀ aryloxy group, an optionally substituted 3- to 12-membered monocyclic or polycyclic heterocyclic oxy group, a carboxyl group, an optionally substituted C₁₋₆ alkylcarbonyl group, an optionally substituted C₃₋₁₀ cycloalkylcarbonyl group, an optionally substituted C₆₋₁₀ arylcarbonyl group, an optionally substituted 3- to 12-membered monocyclic or polycyclic heterocyclic carbonyl group, an optionally substituted C₁₋₆ alkoxycarbonyl group, an optionally substituted C₃₋₁₀ cycloalkoxycarbonyl group, an optionally substituted C₆₋₁₀ aryloxycarbonyl group, an optionally substituted 3- to 12-membered monocyclic or polycyclic heterocyclic oxycarbonyl group, an optionally substituted aminocarbonyl group, an optionally substituted C₁₋₆ alkylthio group, an optionally substituted C₃₋₁₀ cycloalkylthio group, an optionally substituted C₆₋₁₀ arylthio group, an optionally substituted 3- to 12-membered monocyclic or polycyclic heterocyclic thio group, a sulfinic acid group, an optionally substituted C₁₋₆ alkylsulfinyl group, an optionally substituted C₃₋₁₀ cycloalkylsulfinyl group, an optionally substituted C₆₋₁₀ arylsulfinyl group, an optionally substituted 3- to 12-membered monocyclic or polycyclic heterocyclic sulfinyl group, an optionally substituted aminosulfinyl group, a sulfonic acid group, an optionally substituted C₁₋₆ alkylsulfonyl group, an optionally substituted C₃₋₁₀ cycloalkylsulfonyl group, an optionally substituted C₆₋₁₀ arylsulfonyl group, an optionally substituted 3- to 12-membered monocyclic or polycyclic heterocyclic sulfonyl group, or an optionally substituted aminosulfonyl group.

94. The compound or a pharmaceutically acceptable salt, or a solvate thereof of claim 93, wherein R² is an optionally substituted C₁₋₁₀ alkyl group.

95. The compound or a pharmaceutically acceptable salt, or a solvate thereof of claim 93, wherein R² is a methyl group.

96. The compound or a pharmaceutically acceptable salt, or a solvate thereof of claim 93, wherein R³ is an optionally substituted C₁₋₁₀ alkyl group.

97. The compound or a pharmaceutically acceptable salt, or a solvate thereof of claim 93, wherein R³ is a methyl group.

98. The compound or a pharmaceutically acceptable salt, or a solvate thereof of claim 93, wherein R^{4A}, R^{4B}, R^{4C}, and R^{4D} are the same or different, each independently selected from the group consisting of a hydrogen atom, a halogen atom, a cyano group, a hydroxyl group, an optionally substituted amino group, and an optionally substituted C₁₋₆ alkyl group.

99. The compound or a pharmaceutically acceptable salt, or a solvate thereof of claim 93, wherein R^{4A}, R^{4B}, R^{4C}, and R^{4D} are hydrogen atoms.
